(19) United States
(12) Patent Application Publication
(10) Pub. No.: US 2023/0140200 A1
(43) Pub. Date: May 4, 2023

(54) COMPOSITIONS AND METHODS FOR DETECTING CELLS UNDERGOING FERROPTOSIS USING AN ANTIBODY

(71) Applicant: THE TRUSTEES OF COLUMBIA UNIVERSITY IN THE CITY OF NEW YORK, New York, NY (US)

(72) Inventors: Brent R. STOCKWELL, New York, NY (US); Angie Huizhong FENG, New York, NY (US); Jenny JIN, New York, NY (US)

(21) Appl. No.: 17/818,807

(22) Filed: Aug. 10, 2022

Related U.S. Application Data
(63) Continuation-in-part of application No. PCT/US2021/017216, filed on Feb. 9, 2021.
(60) Provisional application No. 62/972,503, filed on Feb. 10, 2020.
(60) Provisional application No. 63/314,962, filed on Feb. 28, 2022.

(51) Int. Cl. C07K 16/28; G01N 33/50

(52) U.S. Cl. CPC C07K 16/2881; G01N 33/5091; C07K 2317/21

(57) ABSTRACT

The present disclosure provides, inter alia, methods for identifying cells undergoing non-apoptotic cell death, e.g., ferroptosis, in a subject, using anti-TfR1 antibodies such as 3F3-FMA. Methods for treating or ameliorating the effects of a cancer in a subject, methods for enhancing the anti-tumor effect of radiation in a subject with cancer undergoing radiotherapy, and compositions and kits comprising anti-TfR1 antibodies disclosed herein are also provided. The present disclosure also provides methods for identifying a classifier that detects and classifies a cell death modality in a cell. The classifier constructed by such methods serves as an unbiased tool for identification of features that best distinguish various cell death modalities. Also provided are methods for detecting a cell death modality in a system such as e.g., a cancer patient receiving therapies.

### CROSS REFERENCE TO RELATED APPLICATIONS

The present application is a continuation-in-part of PCT International Application No. PCT/US2021/017216, filed Feb. 9, 2021, which claims benefit of U.S. Provisional Patent Application Ser. No. 62/972,503, filed on Feb. 10, 2020. The present application also claims benefit of U.S. Provisional Patent Application Ser. No. 63/314,962, filed on Feb. 28, 2022. The aforementioned applications are incorporated by reference herein in their entireties.

### GOVERNMENT FUNDING

This invention was made with government support under grant nos. CA209896 and CA087497, awarded by the National Institutes of Health. The government has certain rights in the invention.

### FIELD OF DISCLOSURE

The present disclosure provides, inter alia, compositions and methods for identifying cells undergoing non-apoptotic cell death in a subject. Compositions and methods for detecting and classifying cell death modalities are also provided.

### INCORPORATION BY REFERENCE OF SEQUENCE LISTING

This application contains references to amino acids and/or nucleic acid sequences that have been filed concurrently herewith as sequence listing XML file “CU20038-US-seq.xml”, file size of 47,658 bytes, created on Aug. 4, 2022. The aforementioned sequence listing is hereby incorporated by reference in its entirety pursuant to 37 C.F.R. § 1.52(e)(5).

### BACKGROUND OF THE DISCLOSURE

Ferroptosis is a regulated form of cell death that involves accumulation of lethal phospholipid peroxides and is suppressed by iron chelators and lipophilic antioxidants (Stockwell et al., 2017). It is characterized by loss of activity of glutathione peroxidase 4 (GPX4), which is the major protein in animals that can reduce lipid hydroperoxides in a membrane phospholipid context (Yang et al., 2014).

Ferroptosis induction has been shown to have potential as a cancer therapeutic strategy. Unlike apoptosis, which most cancer cells can evade, ferroptosis is lethal to many tumor cells that have become dependent on suppression of ferroptosis for their survival, including some of the most drug-resistant and aggressive cancer cells, such as persister cells and cells that have undergone epithelial-mesenchymal transition (EMT) (Hangauer et al., 2017; Viswanathan et al., 2017). Thus, triggering ferroptosis may open up new therapeutic avenues for treating drug-resistant cancers (Hangauer et al., 2017). The first two ferroptosis inducers reported, erastin and RSL3, were identified in phenotypic screens targeting engineered tumor cells (Dixon et al., 2012). To date, four classes of ferroptosis inducers have been discovered (Feng and Stockwell, 2018). Class 1 ferroptosis inducers act through inhibition of system xc−, the transmembrane cystine-glutamate antiporter, which imports cystine into cells. Cystine, the cysteine disulfide, is required for the biosynthesis of glutathione (GSH), which is a cofactor and co-substrate for GPX4. Depletion of GSH leads to loss of GPX4 activity, resulting in accumulation of lethal lipid peroxides and ferroptotic cell death. Erastin and its more potent analogs imidazole ketone erastin (IKE) and piperazine erastin (PE), as well as the clinically used drugs sulfasalazine and sorafenib, belong to this class of inducers.

Class 2 ferroptosis inducers act through direct inhibition of GPX4. (1S, 3R)-RSL3 (henceforth RSL3) covalently interacts with GPX4 and inhibits its enzymatic activity, resulting in ferroptotic cell death (Yang et al., 2014). Class 3 ferroptosis inducers, ferroptosis inducer 56 (FIN56) and caspase-independent lethal 56 (CIL56), deplete GPX4 protein and mevalonate-derived coenzyme Q10, which is an endogenous lipophilic antioxidant that suppresses lipid peroxidation (Shimada et al., 2016). The class 4 ferroptosis inducer, ferroptosis inducer endoperoxide (FINO2), acts by oxidizing iron, driving lipid peroxidation, and indirectly inactivating GPX4 enzymatic function in cells (Abrams et al., 2016; Gaschler et al., 2018).

A number of ferroptosis inhibitors have also been identified. The first class includes iron chelators, which chelate excessive labile iron and prevent lipid peroxidation. The second class constitutes radical-trapping antioxidants, including vitamin E, butylated hydroxytoluene (BHT), ferrostatin-1 (Fer-1), and liproxstatin-1. These agents prevent lipid peroxidation through an H-atom transfer mechanism (Skouta et al., 2014). Other ferroptosis inhibitors include deuterated polyunsaturated fatty acids (D-PUFAs), inhibitors of Acyl-CoA Synthetase Long Chain Family Member 4 (ACSL4), glutaminolysis inhibitors, lipoxygenase inhibitors, cycloheximide, beta-mercaptoethanol, dopamine, selenium, and vildagliptin (Stockwell et al., 2017).

Ferroptosis is implicated in various human diseases and pathologies: it has been found that ferroptosis plays a role in the progression of degenerative diseases of the kidney, heart, liver, and brain (Feng and Stockwell, 2018). Stroke, Alzheimer's disease, Huntington's disease and Parkinson's disease are among candidates for neurodegenerative diseases involving ferroptosis (Weiland et al., 2018).

In order to identify the extent to which ferroptosis occurs in pathological and physiological contexts, it is necessary to identify reagents that selectively label cells undergoing ferroptosis. Three hallmarks of ferroptosis, oxidation of PUFA-PLs, redox-active iron, and loss of lipid peroxide repair capacity, have been used as criteria to measure the extent to which ferroptosis occurs (Dixon and Stockwell, 2019). First, the fluorescent probes C11-BODIPY and LiperFluo are used as indicators of lipid peroxidation. BODIPY-C11 indicates the production of reactive oxygen species (ROS) in a lipophilic environment through a change in fluorescence of the probe. It is sensitive to the free radical species formed from hydroperoxides, but not hydroperoxides themselves (Drummen et al., 2002). In contrast, Liperfluo directly reacts with lipid hydroperoxides to form highly fluorescent Liperfluo-OX, which can be detected at long wavelengths (Yamanaka et al., 2012). Second, an increase in redox-active iron quantitatively determines the ratio of ferrous (Fe²⁺) to ferric (Fe³⁺) iron. Third, loss of lipid peroxide repair is commonly determined by the enzymatic activity of GPX4. NADPH is added as an indicator of the ability of GPX4 to reduce its substrate and cofactor glutathione. However, these experiments are technically challenging, are limited to biochemical assays, and cannot be used in fixed tissue sections.

Accordingly, there is a need for methods to analyze biological samples such as tissue sections from human patients to determine the extent to which cells are undergoing ferroptosis. This disclosure is directed to meeting these and other needs.

### SUMMARY OF THE DISCLOSURE

Ferroptosis is a form of regulated cell death process driven by the iron-dependent accumulation of polyunsaturated-fatty-acid-containing phospholipids (PUFA-PLs). Three key molecular features of ferroptosis are peroxidation of PUFA-PLs, increased redox-active ferrous iron, and defective lipid peroxide repair (Dixon and Stockwell, 2019). However, there is currently no reliable means of selectively staining ferroptotic cells in tissue sections to characterize relevant models and diseases. The present disclosure addresses this gap by generation of ferroptosis-specific antibodies. For example, mice were immunized with membranes from diffuse large B cell lymphoma (DLBCL) cells treated with the ferroptosis inducer piperazine erastin (PE), and approximately 4,750 of the resulting monoclonal antibodies generated were screened. One antibody, termed 3F3 anti-Ferroptotic Membrane Antibody (3F3-FMA), was found to be effective as a selective ferroptotic staining reagent using immunofluorescence (IF). The antigen of 3F3-FMA was identified by immunoprecipitation and mass spectrometry as the human transferrin receptor protein 1 (TfR1), which imports iron from the extracellular environment into cells. This finding was validated with several additional anti-TfR1 antibodies. 3F3-FMA was compared to other potential ferroptosis staining reagents via immunofluorescence staining and visualized by fluorescence microscopy and flow cytometry. It was found that anti-TfR1 and anti-MDA antibodies were effective in reliably staining ferroptotic tumor cells in two human cell line xenograft cancer models. In summary, these findings suggest that TfR1 antibodies can be used as a molecular marker to selectively label cells undergoing ferroptosis. Together, these antibodies allow for the first time the detection of cells undergoing ferroptosis in human tissue sections.

Accordingly, one embodiment of the present disclosure is a method for identifying cells undergoing non-apoptotic cell death in a subject comprising: a) contacting a biological sample from the subject with an anti-TfR1 (transferrin receptor protein 1) antibody; and b) determining whether the anti-TfR1 antibody specifically binds to a cell in the sample, wherein the binding of the antibody to a cell in the sample is indicative of the cell undergoing non-apoptotic cell death.

Another embodiment of the present disclosure is a method for identifying ferroptosis in a subject, comprising: a) obtaining a biological sample from the subject; b) contacting the sample with an anti-TfR1 (transferrin receptor protein 1) antibody; c) carrying out an immunofluorescent assay on the sample; and d) identifying the presence of or absence of ferroptosis by quantifying membrane fluorescence intensity of the sample.

Another embodiment of the present disclosure is a method for identifying cells undergoing ferroptosis in a subject, comprising: a) obtaining a biological sample from the subject; b) contacting the sample with an anti-TfR1 3B8 2A1 antibody and an anti-MDA 1F83 antibody; and c) determining whether the anti-TfR1 3B8 2A1 antibody and the anti-MDA 1F83 antibody selectively bind to a cell in the sample.

A further embodiment of the present disclosure is a method for treating a cancer in a subject, comprising: a) administering to the subject a therapeutically effective amount of an agent that induces ferroptosis; b) obtaining a biological sample from the subject; c) contacting the sample with an anti-TfR1 (transferrin receptor protein 1) antibody; d) determining whether the antibody selectively binds to a cell in the sample; and e) continuing the current treatment if ferroptosis is present, otherwise adjusting the treatment protocol if ferroptosis is absent.

Another embodiment of the present disclosure is a method for treating a cancer in a subject, comprising: a) administering to the subject a therapeutically effective amount of an agent that induces ferroptosis; b) obtaining a biological sample from the subject; c) contacting the sample with an anti-TfR1 3B8 2A1 antibody and an anti-MDA 1F83 antibody, wherein one or both of the antibodies is tagged with one or more fluorescent molecules; d) detecting a fluorescent signal, if present, wherein the presence or absence of ferroptosis is determined by quantifying membrane fluorescence intensity of the sample via flow cytometry and/or fluorescence microscopy; and e) continuing the current treatment if ferroptosis is present, otherwise adjusting the treatment protocol if ferroptosis is absent.

An additional embodiment of the present disclosure is a method for identifying ferroptosis in a cell, comprising: a) contacting the cell with an anti-TfR1 (transferrin receptor protein 1) antibody; b) carrying out an immunofluorescent assay on the cell; and c) identifying the presence or absence of ferroptosis by quantifying membrane fluorescence intensity of the cell.

Another embodiment of the present disclosure is a method for identifying ferroptosis in a cell, comprising: a) contacting the cell with an anti-TfR1 3B8 2A1 antibody and an anti-MDA 1F83 antibody; b) carrying out an immunofluorescent assay on the cell; and c) identifying the presence or absence of ferroptosis by quantifying membrane fluorescence intensity of the cell via flow cytometry and/or fluorescence microscopy.

Still another embodiment of the present disclosure is an isolated monoclonal antibody or antigen binding fragment thereof, comprising a heavy chain variable region and a light chain variable region, comprising: in the heavy chain variable region, the heavy chain complementarity determining regions set forth as SEQ ID NO: 3, SEQ ID NO: 4, and SEQ ID NO: 5, and in the light chain variable region, the light chain complementarity determining regions set forth as SEQ ID NO: 6, SEQ ID NO: 7, and SEQ ID NO: 8. The present disclosure also provides the monoclonal antibody and antigen binding fragment disclosed above.

Another embodiment of the present disclosure is an isolated nucleic acid molecule encoding the antibody or antigen binding fragment disclosed herein.

Another embodiment of the present disclosure is a vector comprising the nucleic acid molecule disclosed herein.

Another embodiment of the present disclosure is a host cell, comprising the nucleic acid molecule disclosed herein or a vector comprising such nucleic acid molecule.

Another embodiment of the present disclosure is a method for treating or ameliorating the effects of a cancer in a subject in need thereof, comprising: a) administering to the subject a therapeutically effective amount of an agent that induces ferroptosis; b) obtaining a biological sample from the subject; c) contacting the sample with an anti-TfR1 (transferrin receptor protein 1) antibody; d) determining whether the antibody selectively binds to a cell in the sample; and e) administering a therapeutically effective amount of radiation to the subject if ferroptosis is present.

Still another embodiment of the present disclosure is a method for enhancing the anti-tumor effect of radiation in a subject with cancer undergoing radiotherapy, comprising: a) obtaining a biological sample from the subject; b) contacting the sample with an anti-TfR1 (transferrin receptor protein 1) antibody; c) determining whether the antibody selectively binds to a cell in the sample; and d) administering to the subject a therapeutically effective amount of an agent that induces ferroptosis if ferroptosis is absent.

A further embodiment of the present disclosure is a composition, comprising an effective amount of the antibody or antigen binding fragment disclosed herein, or a nucleic acid molecule encoding such antibody or antigen binding fragment, and a pharmaceutically acceptable carrier.

Another embodiment of the present disclosure is a method for identifying a classifier that detects and classifies a cell death modality in a cell. The method comprises: (a) treating cultured cells with a select cell death inducer; (b) contacting the treated cells with an agent targeting a biomarker of the cell death modality; (c) carrying out an immunofluorescent assay on the cells and collecting a set of images of various cell regions; (d) analyzing the images to generate features that are statistically significantly correlated to the cell death modality; (e) performing further statistical analysis on the features generated in step (d) to select signature-features that constitute the classifier; and (f) confirming the accuracy of the classifier with a second set of images that are independently collected by repeating steps (a) to (c).

Yet another embodiment of the present disclosure is a method for detecting a cell death modality in a system. The method comprises: (a) obtaining a biological sample from the system and contacting the biological sample with an agent targeting a biomarker of the cell death modality; (b) carrying out an immunofluorescent assay on the biological sample and collecting a set of images of various cell regions; (c) constructing a classifier following the method disclosed herein; and (d) applying the classifier on the set of images collected in step (b) to detect the cell death modality.

### BRIEF DESCRIPTION OF THE DRAWINGS

The application file contains at least one drawing executed in color. Copies of this patent application with color drawing(s) will be provided by the Office upon request and payment of the necessary fee.

FIGS. 1A-1D show a screen of 672 monoclonal antibodies generated by infecting mice with piperazine erastin (PE)-induced membrane fractions (See also FIGS. 7A-7B). In FIG. 1A, cells were confirmed to be undergoing ferroptosis by fluorescent probe C11-BODIPY as a lipid ROS indicator. Blue represents DMSO-treated cells. Red represents PE-treated cells. FIG. 1B provides the Western blot confirmation of plasma membrane and total membrane by organelle markers. The presence of plasma membrane was determined by anti-sodium potassium ATPase antibody, cytosol by anti-GAPDH, ER by anti-PDI and nuclei by anti-Histone H3. FIG. 1C is a flow chart illustrating the screen from ˜4,750 unknown target antibodies to 3F3-FMA by flow cytometry, immunofluorescence and high-content image analysis. In FIG. 1D, 3F3-FMA is shown as an example of cherry picking and high-content-analysis. There was increased number of cells, which had more than three spots in cytoplasm in RSL3-induced ferroptosis. * indicated p value≤0.05. Data plotted are mean±s.e.m.

FIGS. 2A-2D show the identification of 3F3-FMA as a ferroptosis marker using various cell death inducers and different cell lines (See also FIGS. 8A-8C). In FIG. 2A, HT-1080 cells (human fibrosarcoma cells) were incubated with 1 μM RSL3 or 1 μM RSL3+5 μM Fer-1 for 4 h. 3F3-FMA-bound cells showed a significantly different pattern in RSL3-induced ferroptosis, but not in Fer-1 rescued process. Nuclei were stained with DAPI in blue. 3F3-FMA-bound cells were stained with Alexa Fluor 594 in red. White arrows indicated the differences. Quantification of membrane intensities of 3F3 FMA was shown on the right (DMSO, n=107; RSL3, n=96; RSL3+Fer-1 n=123). **** indicated p value≤0.0001, ns indicated p value>0.05 (one-way ANOVA). Data plotted are mean±s.e.m. Each dot represents one cell. In FIG. 2B, HT-1080 cells (human fibrosarcoma cells) were incubated with 10 μM IKE for 8 h, 15 μM erastin for 8 h, 10 μM FIN56 for 8 h, 15 μM FINO₂ for 8 h and 100 μM tBuOOH for 8 h. 3F3-FMA-bound cells showed different staining patterns in IKE, erastin, FIN56, FINO₂ and tBuOOH-induced ferroptosis. Nuclei were stained with DAPI in blue. 3F3-FMA-bound cells were stained with Alexa Fluor 594 in red. White arrows indicated the differences. Quantification of membrane intensities of 3F3-FMA was shown on the bottom (IKE, n=113 and 129; erastin, n=68 and 92; FIN56, n=68 and 86; FINO2, n=59 and 30; tBuOOH, n=73 and 49). **** indicated p value≤0.0001 (two-tailed t-test). Data plotted are mean±s.e.m. Each dot represents one cell. In FIG. 2C, HT-1080 cells (human fibrosarcoma cells) were incubated with 1 μM staurosporine (STS) for 6 h and 2 μM camptothecin for 24 h. Cleaved caspase-3 antibody and cleaved PARP antibody were used to mark the induction of apoptosis. The staining pattern of 3F3-FMA-bound cells in apoptosis was different from ferroptosis. Nuclei were stained with DAPI in blue. 3F3-FMA-bound cells were stained with Alexa Fluor 594 in red. Quantification of membrane intensity of 3F3-FMA (DMSO, n=71, STS, n=53; DMSO, n=166, camptothecin, n=91) and overall intensity of cleaved caspase-3 (n=7) and cleaved PARP (n=7) is shown on the right. * indicated p value≤0.05, *** indicated p value≤0.001. Data plotted are mean±s.e.m. Each dot represents one cell. In FIG. 2D, A-673 cells, SK-BR-3 cells, Huh-7 cells, and SK-LMS-1 cells were incubated with 1 μM RSL3 for 4 h. The same pattern as with 3F3 FMA was observed. Nuclei were stained with DAPI in blue. 3F3 FMA was stained with Alexa Fluor 594 in red. White arrows indicated the differences. Quantification of membrane intensities of 3F3 FMA was shown on the right (A-673, n=70 and 16; SK-BR-3, n=65 and 33; Huh-7, n=63 and 61; SK-LMS-1, n=149 and 139). **** indicated p value≤0.0001 (two-tailed t-test). Data plotted are mean±s.e.m. Each dot represents one cell.

FIGS. 3A-3F show that the target of the 3F3-FMA monoclonal antibody is the transferrin receptor protein 1, which is located in the Golgi and the plasma membrane (see also FIG. 9). FIG. 3A shows the IP-MS result of a human TfR1 sequence. The yellow highlight represents the identified sequence. Green indicates modified amino acids (M: oxidation of methionine and N: deamination of asparagine). The sequence coverage was 53%. In FIG. 3B, 10 μM of siTfR1 and siNT were combined with Lipofectamine RNAiMAX in Opti-Mem media for 48 h. Then, HT-1080 cells were reseeded in regular media for additional 24 h. Cells were incubated with 1 μM RSL3 for 4 h, and then were fixed, permeabilized and stained for DAPI (nuclei, blue) and 3F3 FMA (red). No 3F3-FMA was detected in siRNA knockdown of transferrin receptor. siNT was used as control. White arrows indicate the absence. In FIG. 3C, HT-1080 cells were incubated with 1 μM RSL3 for 4 h, and then were fixed, permeabilized and stained with DAPI (nuclei, blue), GM130 (Golgi, green) and 3F3-FMA. 3F3-FMA co-localized with the Golgi complex. White arrows indicate the overlap. Green arrows indicate the bright dots of 3F3-FMA in normal condition. In FIG. 3D, HT-1080 cells were incubated with 1 μM RSL3 for 4 h, and then were fixed and stained with DAPI (nuclei, blue), WGA (plasma membrane, red) and 3F3-FMA or TfR1 3B8 2A1 (green). 3F3-FMA and TfR1 3B8 2A1 co-localized with the plasma membrane during RSL3-induced ferroptosis. White arrows indicate the overlap. In FIG. 3E, HT-1080 cells were incubated with 1 μM RSL3 for 4 h and were collected at 0 h, 0.5 h, 1 h, 2 h, 3 h, and 4 h time points. Cells were then fixed, permeabilized and stained with DAPI (nuclei, blue), GM130 (Golgi, green) and 3F3-FMA. White arrows indicate the accumulation of TfR1 in the plasma membrane, while green arrows indicate the overlap area with the Golgi. More membrane-located TfR1 and less Golgi-located TfR1 were observed. Quantification of membrane intensities of 3F3-FMA and co-localization of the Golgi marker GM130 and 3F3-FMA are shown in the bottom. **** indicates p value≤0.0001, ns indicates p value>0.05 (two-tailed t-test). Data plotted are mean±s.e.m. Each dot represents one cell. In FIG. 3F, HT-1080 cells were incubated with 1 μM RSL3 for 4 h or 5 μM IKE for 18 h, and then were fixed and stained with DAPI (nuclei, blue) and 3F3-FMA (red). Without permeabilization, 3F3-FMA stained the cell surface clearly for cells undergoing ferroptosis. White arrows indicate the boundaries.

FIGS. 4A-4C show that 3F3-FMA, anti-TfR1 3B8 2A1, anti-TfR1 H68.4, anti-MDA 1F83 and anti-4-HNE antibodies could be used as ferroptosis markers by immunofluorescence (See also FIGS. 10A-10B). In FIG. 4A, HT-1080 cells were incubated with 1 μM RSL3 for 4 h, and then were fixed, permeabilized and stained with DAPI (nuclei, blue), 3F3-FMA, anti-TfR1 3B8 2A1 or anti-TfR1 H68.4 antibodies (red). White arrows indicate the differences. Quantification of membrane intensities of anti-TfR1 antibodies are shown at the bottom (3F3-FMA, n=107 and 96; TfR1 3B8 2A1, n=32 and 9; TfR1 H68.4, n=22 and 13). **** indicated p value≤0.0001 (two-tailed t-test). Data plotted are mean±s.e.m. Each dot represents one cell. In FIG. 4B, HT-1080 cells were incubated with 1 μM RSL3 for 4 h, and then were fixed, permeabilized and stained with DAPI (nuclei, blue), anti-MDA 1F83 antibody (red) and anti-4-HNE ab46545 antibodies (green). White arrows indicated the differences. Quantification of membrane intensities of the antibodies are shown at the bottom (MDA 1F83, n=35 and 73; 4-HNE ab46545, n=33 and 26). **** indicated p value≤0.0001 (two-tailed t-test). Data plotted are mean±s.e.m. Each dot represents one cell. In FIG. 4C, HT-1080 cells were incubated with 1 μM STS for 6 h, and then were fixed, permeabilized and stained with DAPI (nuclei, blue), 3F3-FMA (red), anti-TfR1 3B8 2A1 (red), anti-TfR1 H68.4 (red), anti-MDA 1F83 (red) and anti-4-HNE ab46545 antibodies (green). White arrows indicated the stain out of intact cells. Quantification of membrane intensities of the antibodies are shown at the bottom (3F3 FMA, n=71 and 53; TfR1 3B8 2A1, n=103 and 63; TfR1 H68.4, n=93 and 80; MDA 1F83, n=81 and 71; 4-HNE ab46545, n=56 and 69). ns indicated p value>0.05 (two-tailed t-test). Data plotted are mean±s.e.m. Each dot represents one cell.

FIGS. 5A-5D show that anti-TfR1, anti-MDA and anti-4-HNE antibodies worked in flow cytometry but not in western blot. In FIG. 5A, HT-1080 cells were treated with DMSO or 1 μM RSL3 for 4 h. Cells were then harvested and stained with 1^{st} and 2^{nd} antibodies or C11-BODIPY without permeabilization. Around 15,000 cells were recorded and gated. RSL3-treated cells had increased intensities of 3F3 FMA, TfR1 3B8 2A1, TfR1 H68.4, MDA 1F83, MDA ab6463, and 4-HNE ab46545. C11-BODIPY, a probe for lipid peroxidation, was used as a metric. In FIG. 5B, HT-1080 cells were treated with DMSO or 1 μM STS for 6 h. Cells were then harvested and stained with 1^{st} and 2^{nd} antibodies without permeabilization. ˜50,000 cells were recorded and gated. STS-treated cells had decreased intensities of 3F3-FMA staining. In FIG. 5C, HT-1080 cells were treated with 1 μM RSL3 for 2 h and 10 μM IKE for 4 h. Cells were collected at multiple time points shown in the figure. Cells were then lysed, stained with 1^{st} and 2^{nd} antibodies and detected using western blot. 3F3-FMA and TfR1 H68.4 were used as TfR1 antibodies. An increased amount of TfR1 protein was observed during ferroptosis. GAPDH was used as control. In FIG. 5D, HT-1080 cells were treated with 1 μM RSL3 or 10 μM IKE for 8 h. cDNAs were generated from total RNA collected and purified from cells. Two sets of TfR1 primers were used to quantify the amount of cellular TfR1 transcripts. CHAC1 was used as positive control. The level of TfR1 mRNAs didn't increase during ferroptosis. Blotting of TfR1 proteins using TfR1 H68.4 antibody is shown side by side. 4 h and 8 h of RSL3-treated western blot weren't harvested due to an insufficient number of viable cells.

FIGS. 6A-6C show the comparison of TfR1 antibodies and other potential ferroptosis staining reagents in mouse xenograft tumor tissue samples (See also FIGS. 11A-11B). FIG. 6A is an illustration of the preparation of the mouse xenograft tumor and IKE dosage. In FIG. 6B, B cell lymphoma tumor tissues were fixed in 4% PFA for 24 h, perfused in 30% sucrose for 24 h, and stained with 1^{st} and 2^{nd} antibodies. Anti-TfR1 3B8 2A1, anti-TfR1 H68.4 and anti-MDA 1F83 showed significant difference of intensities between vehicle and IKE treatments. 3F3-FMA showed no difference. anti-MDA ab6463 and anti-4-HNE ab46545 showed slight differences. Controls without primary antibody staining are shown on the right. Quantification of overall intensity of the antibodies is shown in the bottom (n=7). **** indicates p value≤0.0001, *** indicates p value≤0.001, ** indicates p value≤0.01, * indicates p value≤0.05, and ns indicates p value>0.05 (two-tailed t-test). Data plotted are mean±s.e.m. Each dot represents one image. In FIG. 6C, HCC tumor tissues was fixed in 4% PFA for 24 h, perfused in 30% sucrose for 24 h, and stained with 1^{st} and 2^{nd} antibodies. 3F3-FMA, anti-TfR1 3B8 2A1 and anti-MDA 1F83 showed difference in intensities between vehicle and IKE groups, while other antibodies didn't. Controls without primary antibody staining are shown on the right. Quantification of overall intensity of the antibodies is shown in the bottom (n=7). **** indicates p value≤0.0001, ** indicates p value≤0.01, and ns indicates p value>0.05 (two-tailed t-test). Data plotted are mean±s.e.m. Each dot represents one image.

FIGS. 7A-7B show the high-content analysis sequence of 3F3 FMA as an example. In FIG. 7A, HT-1080 cells were seeded in 384-well plates and treated with 0.3 μM RSL3 for 2.5 h. Then the cells were fixed with 4% PFA, permeabilized with 0.5% Tween-20 and stained with primary antibody and secondary antibody. The cells were then stained with Hoechst 33342 (nuclei detection) and Phalloidin-TRITC (cytoplasmic region detection) prior to recording by automated Operetta® microscope. Multiparametric image analysis was performed using Columbus Software 2.8.0 (PerkinElmer). The cell population for analysis was selected by detection and segmentation of nuclei and corresponding cytoplasm. Border objects and small cells were removed from analysis. The cells highlighted green in graph 6 were selected for further analysis. In FIG. 7B, selected cells from (A) and the corresponding cell regions were used for Alexa488 intensity and spot detection. For 3F3-FMA, the number of cells with more than 3 spots in the cytoplasm (green cells in graph 3) was quantified.

FIGS. 8A-8C show that nuclei are smaller in ferroptotic cells. In FIG. 8A, HT-1080 cells were incubated with 10 μM IKE and 5 μM Fer-1 for 8 h. Nuclei were stained with DAPI in blue. 3F3 FMA was stained with Alexa Fluor 594 in red. 3F3-FMA-bound cells showed significantly different patterns in IKE-induced ferroptosis, but not in Fer-1 rescued process. White arrows indicated the differences. Quantification of membrane intensities of 3F3-FMA was shown in the bottom right panel (DMSO, n=68; IKE, n=145; IKE+Fer-1, n=98). **** indicated p value≤0.0001, ns indicated p value>0.05 (one-way ANOVA). Data plotted are mean±s.e.m. Each dot represents one cell. In FIG. 8B, HT-1080 cells in plates 1-3 were treated with DMSO and cells in plates 4-6 were treated with RSL3. Nucleus areas were identified and measured. The average nucleus area for the DMSO group was 240 μm² while the average nucleus area for the RSL3 group was 200 μm² with a decrease of 17%. In FIG. 8C, HT-1080 cells were incubated with 1 μM RSL3 for 4 h, 10 μM IKE for 8 h, 15 μM erastin for 8 h, 10 μM FIN56 for 8 h and 15 μM FINO₂ for 8 h. Nuclei were stained by DAPI and identified using CellProfiler 3.1.8. Mean areas of nuclei for each cell were then calculated. Quantification of nucleus area was shown (DMSO, n=107; RSL3, n=96; DMSO, n=113, IKE, n=129; DMSO, n=68, erastin, n=92; DMSO, n=68, FIN56, n=86; DMSO, n=59, FINO₂, n=30). NuleaData plotted are mean±s.e.m. The nuclei shrank by 27% in 4 h incubation of 1 μM RSL3, 12% in 8 h incubation of 10 μM IKE, 21% in 8 h incubation of 15 μM erastin, 17% in 8 h incubation of 10 μM FIN56 and 49% in 8 h incubation of 15 μM FINO₂.

FIG. 9 shows that the target of 3F3 FMA was not in mitochondria or ER. HT-1080 cells were incubated with 1 μM RSL3 for 4 h, and then were fixed, permeabilized and stained with DAPI (nuclei, blue), and either Tom20 (mitochondria marker, green) or PDI (ER marker, green), and also 3F3 FMA. 3F3 FMA didn't co-localize with these mitochondria and ER markers.

FIGS. 10A-10C show that 3F3 FMA, anti-TfR1 3B8 2A1, anti-TfR1 H68.4, anti-MDA 1F83 and anti-4-HNE antibodies could be used as ferroptosis marker by immunofluorescence. In FIG. 10A, HT-1080 cells were incubated with 1 μM RSL3 for 4 h, and then were fixed, permeabilized and stained with DAPI (nuclei, blue), anti-TfR1 D7G9X (green), anti-MDA ab6463 (green) and anti-ACSL4 sc-365230 antibodies (red). Quantification of membrane intensities of the antibodies is shown at the bottom (TfR1 D7G9X, n=23 and 6; MDA ab6463, n=49 and 27; ACSL4 sc-365230, n=69 and 41. * indicated p value≤0.05 and ns indicated p value>0.05 (two-tailed t-test). Data plotted are mean±s.e.m. Each dot represents one cell. In FIG. 10B, HT-1080 cells were incubated with 2 μM camptothecin for 24 h, and then were fixed, permeabilized and stained with DAPI (nuclei, blue), 3F3 FMA (red), anti-TfR1 3B8 2A1 (red), anti-TfR1 H68.4 (red), anti-MDA 1F83 (red) and anti-4-HNE ab46545 antibodies (green). Quantification of membrane intensities of the antibodies is shown at the bottom (3F3 FMA, n=166 and 91; anti-TfR1 3B8 2A1, n=164 and 116; anti-TfR1 H68.4, n=186 and 112; anti-MDA 1F83, n=109 and 119; 4-HNE ab46545, n=122 and 74). Data plotted are mean±s.e.m. Each dot represents one cell. In FIG. 10C, HT-1080 cells were incubated with 1 mM H₂O₂ for 4 h to induce oxidative stress independent of ferroptosis, and then were fixed, permeabilized and stained with DAPI (nuclei, blue), 3F3-FMA (red), anti-TfR1 3B8 2A1 (red), anti-TfR1 H68.4 (red), anti-MDA 1F83 (red), and anti-4-HNE ab46545 antibodies (green). Quantification of membrane intensities and overall intensities of the antibodies is shown on the bottom (3F3-FMA, n=62 and 73; anti-TfR1 3B8 2A1, n=43 and 38; anti-TfR1 H68.4, n=64 and 64; anti-MDA 1F83, n=45 and 46; 4-HNE ab46545, n=53 and 62). **** indicated p value≤0.0001, ns indicated p value>0.05 (two-tailed t-test). Data plotted are mean±s.e.m. Each dot represents one cell.

FIGS. 11A-11B show that diffuse large B cell lymphoma and hepatocellular carcinoma mouse xenograft tissues contain tumor cells but not immune cells. In FIG. 11A, B cell lymphoma tumor tissues were fixed in 4% PFA for 24 h, perfused with 30% sucrose for 24 h, and stained with primary and secondary antibodies, as indicated. Staining of CD20, a B cell lymphoma marker was positive, while staining of CD8 and CD45, immune cell markers, was negative, indicating that tumor cells, but not infiltrating immune cells, were present in the B cell lymphoma tissue samples. Representative images are shown. In FIG. 11B, hepatocellular carcinoma (HCC) xenograft tumor tissues was fixed in 4% PFA for 24 h, perfused with 30% sucrose for 24 h, and stained with primary and secondary antibodies. Staining of GPC3, an HCC marker, was positive, while staining of CD8 and CD45 was negative, indicating that tumor cells, but not these infiltrating immune cells, were present in the HCC tissue samples. Representative images are shown.

FIGS. 12A-12B show 3F3 FMA staining in human Huntington's disease brains and mouse tissues. In FIG. 12A, human HD and control brain tissues were fixed in 4% PFA for 24 h, perfused in 30% sucrose for 24 h, and stained with DAPI (nuclei, blue) and 3F3 FMA (red). The expression level of TfR1 is low. There was no difference between HD and control groups. In FIG. 12B, mouse liver tissues and mouse GBM tissues were fixed and stained with DAPI (nuclei, blue) and 3F3 FMA (red). 3F3 FMA was able to recognize mouse TfR1.

FIG. 13 shows that EGFR was internalized during ferroptosis. HT-1080 cells were incubated with 1 μM RSL3 in serum-free medium for 4 h (ferroptosis induction and serum starvation), and were incubated with 25 ng/mL EGF for 40 min. Cells were then fixed, permeabilized and stained with DAPI (nuclei, blue) and EGFR (red). EGFR was internalized in the presence of EGF during ferroptosis, indicating that clathrin-mediated endocytosis wasn't affected in ferroptosis. The shrinking nuclei indicated that ferroptosis was happening.

FIG. 14A shows the map of pcDNA3.1(+)-scFv, the sequence information of which is set forth as SEQ ID No: 24.

FIG. 14B shows the map of pET28a(+)-scFv, the sequence information of which is set forth as SEQ ID No: 25.

FIGS. 15A-15B are images undergoing different cell death modalities for machine learning analysis. In FIG. 15A, HT-1080 cells were incubated with ferroptosis inducers RSL3 (1 μM) or IKE (20 μM), apoptosis inducer STS (1 μM), or DMSO control. Nuclei were stained with DAPI (blue). TfR1 was labeled with 3F3-FMA and Alexa Fluor 594 secondary antibody (red). F-actin was labeled with FITC-phalloidin (green). Images were captured using a Zeiss LSM800 confocal microscope at 63×/1.40 oil DIC objective. For each treatment, representative images from the training data set are depicted. As shown in FIG. 15B, in parallel with the immunofluorescence experiments, CellTiter-Glo viability assays were used to monitor the percentage cell death for each treatment, and cells were fixed when percentage cell death reached 10-20%. The concentrations and time points that resulted in this extent of cell death in each set are listed for each treatment.

FIGS. 16A-16C show the results of feature extraction and classifier discovery. In FIG. 16A, the experiment consisted of 120 images per condition (DMSO, IKE, RSL3, STS). The image analysis software extracted 1,473 features for the blue, green and red fluorescence signals. The features can roughly be grouped in intensity, morphology/symmetry and texture features. Undefined values (NaN, “Not a Number) FIG. 16B shows the principal component analysis of 1,373 features extracted from the images. Individual images are visualized as points on the scatter plot of the first two principal components. The color code is according to the treatment label (Red=DMSO, blue=RSL3, green=IKE and purple=STS) and was added after the PCA was conducted. FIG. 16C is the feature matrix of the training data set (scaled for visualization purpose) is cleared for highly correlated features (‘included’) and informative features are isolated by pairwise logistic lasso regressions (‘selected’). Finally, a multinomial logistic lasso regression model is fitted to the reduced feature matrix and a classifier is identified (‘classifier’: 23 features with corresponding regression model coefficients). blgr=bluegreen.

FIGS. 17A-17D show the results of model validation. As shown in FIG. 17A, the classifier was applied to the independent test data set for model validation. FIG. 17B shows the comparison of the known class with the predicted class measures classifier performance. Each class is enriched in the corresponding samples, thereby validating the model. FIGS. 17C and 17D are confusion tables for the multiclass prediction. In FIG. 17C, DMSO, IKE+RSL3 and STS classes are predicted with an accuracy of 93%. In FIG. 17D, DMSO, IKE, RSL3 and STS are predicted with 94% accuracy, when IKE and RSL3 are combined.

FIG. 18 shows a pilot study using CellTiter-Glo viability assay to assess optimal cell death inducer concentration and timepoint. HT-1080 cells were treated with ferroptosis inducers RSL3 (1 μM) or IKE (20 μM), apoptosis inducer STS (1 μM), or DMSO vehicle control, at various timepoints. Using a CellTiter-Glo viability assay, an optimal treatment timepoint (identified above by the highlighted rows) was determined for each treatment when percentage cell death reached between 10-20%.

FIG. 19 illustrates a workflow of the automated image analysis and data extraction. In each image, the nuclei were identified using DAPI staining. The cytoplasm and membrane region were segmented using phalloidin-FITC staining. Subsequently, a large number of features (intensity, morphology/symmetry, texture) were extracted for each cell segment by processing and analyzing the blue-green and red fluorescence signals. All data were then combined and used for further analyzes. For detailed description see material and methods section.

FIGS. 20A-20D are boxplots of representative features. Each dot shows (A) the sum or (B-C) the median value of an image feature. The data were sorted by treatment (DMSO, IKE, RSL3, STS). (FIG. 20A) Number of Nuclei; (FIG. 20B) Nucleus Blue Intensity; (FIG. 20C) Nucleus Roundness; (FIG. 20D) Membrane region red intensity.

FIGS. 21A-21B show CTG data and visualization of the extracted data for validation experiment (test data set): in FIG. 21A, CTG values for the different treatments that were generated in parallel to the immunofluorescent staining. FIG. 21B shows the principal component analysis of validation experiment. PC1=29.74% and PC2=7.96%.

FIG. 22 shows signature of features in cell death classification.

FIG. 23 is a list of features highly correlated to features in signature.

FIG. 24 is a schematic showing that machine learning classifies ferroptosis and apoptosis cell death modalities with TfR1 immunostaining.

### DETAILED DESCRIPTION OF THE DISCLOSURE

It is believed that a ferroptosis-specific antibody would facilitate examining the consequences of ferroptosis in a variety of contexts, including tissue sections, as well as cells in culture. Several antigens have been proposed as potential indicators of ferroptosis.

PTGS2 mRNA, encoding cyclooxygenase-2 (COX-2), was the most upregulated gene in BJeLR cells upon treatment with either erastin or RSL3 in a survey of 83 oxidative stress genes (Yang et al., 2014). CHAC1 mRNA (cation transport regulator homolog 1), an ER stress response gene, was found to be upregulated during the inhibition of system xc− (Dixon et al., 2014). RT-qPCR is used to measure the mRNA level of PTGS2 and CHAC1 in cells. However, detecting ferroptosis using antibodies against the proteins encoded by these genes has proved challenging; moreover, CHAC1 mRNA is primarily upregulated by system xc− inhibitors, but not by other ferroptosis inducers, and PTGS2 is upregulated in other contexts.

Acyl-CoA synthetase long-chain family member 4 (ACSL4) was found to be required for ferroptotic cell death (Dixon et al., 2015; Doll et al., 2017; Yuan et al., 2016). The expression of ACSL4 was downregulated in ferroptosis-resistant cells compared to ferroptosis-sensitive cells (Yuan et al., 2016). However, it is not clear whether the expression level of ACSL4 changes for ferroptosis-sensitive cells undergoing ferroptosis (Muller et al., 2017).

MDA (malondialdehyde) and 4-HNE (4-hydroxynonenal) are aldehydic secondary products of lipid peroxidation. Antibodies against these species and their protein adducts are candidate ferroptosis markers. The anti-MDA 1F83 antibody was raised to target malondialdehyde-modified proteins (Yamada et al., 2001). It has been used as a ferroptosis marker in tissue sections from a mouse lymphoma xenograft model (Zhang et al., 2019). However, these species are also markers of oxidative stress, and may not be specific for ferroptosis compared to other oxidative stress contexts. Therefore, additional specific antibodies for ferroptosis are needed.

In the present disclosure, we report on production of an untargeted pool of monoclonal antibodies from the spleens of mice that were challenged with the membrane fractions of cells induced to undergo ferroptosis with piperazine erastin (PE), an erastin analog and system xc− inhibitor. After screening of ˜4,750 monoclonal antibodies by flow cytometry, 672 antibodies were selected as candidates. A second-round of screening using immunofluorescence resulted in selection of three antibodies as candidates. The 3F3 anti-ferroptotic membrane antibody (3F3-FMA) was selected in a third-round screen of the three hits using immunofluorescence.

The selectivity of 3F3-FMA was validated by treating cells with various ferroptosis inducers and inhibitors, as well as in a comparison with apoptosis inducers. We also tested the 3F3-FMA antibody in several cancer cell lines, and identified the antigen of 3F3-FMA as transferrin receptor protein 1 (TfR1) by immunoprecipitation and mass spectrometry. TfR1 imports iron from the extracellular environment into cells, contributing to the cellular iron pool required for ferroptosis (Yang and Stockwell, 2008). We found that 3F3-FMA was located at the plasma membrane and in the Golgi by co-localization with organelle markers. We compared 3F3-FMA staining with staining by three other anti-TfR1 antibodies, as well as anti-MDA, anti-4-HNE and anti-ACSL4 antibodies, to assess their scope of applications. We found that anti-TfR1 3B8 2A1, anti-TfR1 H68.4, anti-MDA 1F83 and anti-4-HNE ab46545 could detect ferroptotic cells in culture by immunofluorescence. Flow cytometry was sensitive enough to detect the difference between RSL3-treated and DMSO-treated groups for all tested antibodies. Anti-TfR1 3B8 2A1 and anti-MDA 1F83 antibodies provided robust results in mouse xenograft tumor tissue sections. In summary, these findings suggest that anti-TfR1 antibodies, including 3F3-FMA antibody, can be used to directly and selectively label cells undergoing ferroptosis in cell culture and tissue samples. A combination of anti-TfR1 and anti-MDA antibodies is hence proposed to detect ferroptotic cells in human tissue sections. The discovery of TfR1 as a ferroptosis marker implies that TfR1 has a key role in ferroptosis, shedding new light on the mechanisms of ferroptosis.

Accordingly, one embodiment of the present disclosure is a method for identifying cells undergoing non-apoptotic cell death in a subject comprising: a) contacting a biological sample from the subject with an anti-TfR1 (transferrin receptor protein 1) antibody; and b) determining whether the anti-TfR1 antibody specifically binds to a cell in the sample, wherein the binding of the antibody to a cell in the sample is indicative of the cell undergoing non-apoptotic cell death.

In some embodiments, the non-apoptotic cell death is ferroptosis. As used herein, “ferroptosis” means regulated cell death that is iron-dependent. Ferroptosis is characterized by the overwhelming, iron-dependent accumulation of lethal lipid reactive oxygen species. (Dixon et al., 2012) Ferroptosis is distinct from apoptosis, necrosis, and autophagy. (Id.) Assays for ferroptosis are as disclosed herein, for instance, in the Examples section.

Another embodiment of the present disclosure is a method for identifying ferroptosis in a subject, comprising: a) obtaining a biological sample from the subject; b) contacting the sample with an anti-TfR1 (transferrin receptor protein 1) antibody; c) carrying out an immunofluorescent assay on the sample; and d) identifying the presence of or absence of ferroptosis by quantifying membrane fluorescence intensity of the sample.

In some embodiments, the quantification step is carried out by flow cytometry and/or fluorescence microscopy. In some embodiments, the biological sample is a tissue section, a biopsy, blood, or other appropriate bodily fluid.

In some embodiments, the anti-TfR1 antibody targets ectodomains of TfR1. In some embodiments, the anti-TfR1 antibody is selected from 3F3 anti-ferroptotic membrane antibody (3F3-FMA), anti-TfR1 3B8 2A1 antibody, anti-TfR1 H68.4 antibody, and combinations thereof.

In some embodiments, the method disclosed herein further comprises contacting the sample with at least one second antibody. In some embodiments, the at least one second antibody is selected from the group consisting of an anti-MDA (malondialdehyde) antibody, an anti-4-HNE (4-hydroxynonenal) antibody, an anti-ACSL4 (acyl-CoA synthetase long-chain family member 4) antibody, and combinations thereof. In some embodiments, the at least one second antibody is selected from anti-MDA 1F83 antibody, anti-4-HNE ab46545 antibody, and combinations thereof.

In some embodiments, the subject is suffering from a disease associated with dysregulation of non-apoptotic cell death, such as ferroptosis. In some embodiments, the disease is a cancer selected from the group consisting of brain cancer, breast cancer, colon cancer, liver cancer, sarcoma, leiomyosarcoma, hepatocyte-derived carcinoma, fibrosarcoma, glioblastoma, and lymphoma.

In some embodiments, the method disclosed herein further comprises treating the subject identified as having cells undergoing non-apoptotic cell death or ferroptosis by administering to the subject a ferroptosis modulator selected from the group consisting of erastin, imidazole ketone erastin (IKE), piperazine erastin (PE), sulfasalazine, sorafenib, RSL3, ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof.

As used herein, the terms “modulate”, “modulating”, “modulator” and grammatical variations thereof mean to change, such as increasing, decreasing or reducing the occurrence of ferroptosis. In the present disclosure, “contacting” means bringing the compound and optionally one or more additional therapeutic agents into close proximity to the sample such as cells in need of such modulation. This may be accomplished using conventional techniques of drug delivery to the subject or in the in vitro situation by, e.g., providing the compound and optionally other therapeutic agents to a culture media in which the cells are located.

Another embodiment of the present disclosure is a method for identifying cells undergoing ferroptosis in a subject, comprising: a) obtaining a biological sample from the subject; b) contacting the sample with an anti-TfR1 3B8 2A1 antibody and an anti-MDA 1F83 antibody; and c) determining whether the anti-TfR1 3B8 2A1 antibody and the anti-MDA 1F83 antibody selectively bind to a cell in the sample.

A further embodiment of the present disclosure is a method for treating a cancer in a subject, comprising: a) administering to the subject a therapeutically effective amount of an agent that induces ferroptosis; b) obtaining a biological sample from the subject; c) contacting the sample with an anti-TfR1 (transferrin receptor protein 1) antibody; d) determining whether the antibody selectively binds to a cell in the sample; and e) continuing the current treatment if ferroptosis is present, otherwise adjusting the treatment protocol if ferroptosis is absent.

In some embodiments, the cancer is selected from the group consisting of brain cancer, breast cancer, colon cancer, liver cancer, sarcoma, leiomyosarcoma, hepatocyte-derived carcinoma, fibrosarcoma, glioblastoma, and lymphoma.

In some embodiments, the anti-TfR1 antibody is selected from 3F3 anti-ferroptotic membrane antibody (3F3-FMA), anti-TfR1 3B8 2A1 antibody, anti-TfR1 H68.4 antibody, and combinations thereof.

In some embodiments, the method disclosed herein further comprises contacting the sample with at least one second antibody. In some embodiments, the at least one second antibody is selected from anti-MDA 1F83 antibody, anti-4-HNE ab46545 antibody, and combinations thereof.

In some embodiments, the agent that induces ferroptosis is selected from the group consisting of erastin, imidazole ketone erastin (IKE), piperazine erastin (PE), sulfasalazine, sorafenib, RSL3, ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof.

In some embodiments, one or more of the antibodies, including the first and/or second antibodies, are tagged with a detectable label. In some embodiments, the detectable label is selected from fluorescent molecules, radioisotopes, enzymes, antibodies, linkers and combinations thereof. In some embodiments, the detectable label is a fluorescent molecule and determining whether the antibody selectively binds to the cell is carried out by quantifying membrane fluorescence intensity of the sample via flow cytometry and/or fluorescence microscopy.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

Another embodiment of the present disclosure is a method for treating a cancer in a subject, comprising: a) administering to the subject a therapeutically effective amount of an agent that induces ferroptosis; b) obtaining a biological sample from the subject; c) contacting the sample with an anti-TfR1 3B8 2A1 antibody and an anti-MDA 1F83 antibody, wherein one or both of the antibodies is tagged with one or more fluorescent molecules; d) detecting a fluorescent signal, if present, wherein the presence or absence of ferroptosis is determined by quantifying membrane fluorescence intensity of the sample via flow cytometry and/or fluorescence microscopy; and e) continuing the current treatment if ferroptosis is present, otherwise adjusting the treatment protocol if ferroptosis is absent.

An additional embodiment of the present disclosure is a method for identifying ferroptosis in a cell, comprising: a) contacting the cell with an anti-TfR1 (transferrin receptor protein 1) antibody; b) carrying out an immunofluorescent assay on the cell; and c) identifying the presence or absence of ferroptosis by quantifying membrane fluorescence intensity of the cell.

In some embodiments, the quantification step is carried out by flow cytometry and/or fluorescence microscopy.

In some embodiments, the anti-TfR1 antibody targets ectodomains of TfR1. In some embodiments, the anti-TfR1 antibody is selected from 3F3 anti-ferroptotic membrane antibody (3F3-FMA), anti-TfR1 3B8 2A1 antibody, anti-TfR1 H68.4 antibody, and combinations thereof.

In some embodiments, the method disclosed herein further comprises contacting the cell with at least one second antibody. In some embodiments, the at least one second antibody is selected from the group consisting of an anti-MDA (malondialdehyde) antibody, an anti-4-HNE (4-hydroxynonenal) antibody, an anti-ACSL4 (acyl-CoA synthetase long-chain family member 4) antibody, and combinations thereof. In some embodiments, the at least one second antibody is selected from anti-MDA 1F83 antibody, anti-4-HNE ab46545 antibody, and combinations thereof.

In some embodiments, the cell is a cancer cell. In some embodiments, the cancer is selected from the group consisting of brain cancer, breast cancer, colon cancer, liver cancer, sarcoma, leiomyosarcoma, hepatocyte-derived carcinoma, fibrosarcoma, glioblastoma, and lymphoma.

Another embodiment of the present disclosure is a method for identifying ferroptosis in a cell, comprising: a) contacting the cell with an anti-TfR1 3B8 2A1 antibody and an anti-MDA 1F83 antibody; b) carrying out an immunofluorescent assay on the cell; and c) identifying the presence or absence of ferroptosis by quantifying membrane fluorescence intensity of the cell via flow cytometry and/or fluorescence microscopy.

In some embodiments, the cell is a mammalian cell. Preferably, the mammalian cell is obtained from a mammal selected from the group consisting of humans, primates, farm animals, and domestic animals. More preferably, the mammalian cell is a human cancer cell.

Still another embodiment of the present disclosure is an isolated monoclonal antibody or antigen binding fragment thereof, comprising a heavy chain variable region and a light chain variable region, comprising: in the heavy chain variable region, the heavy chain complementarity determining regions set forth as SEQ ID NO: 3, SEQ ID NO: 4, and SEQ ID NO: 5, and in the light chain variable region, the light chain complementarity determining regions set forth as SEQ ID NO: 6, SEQ ID NO: 7, and SEQ ID NO: 8. The present disclosure also provides the monoclonal antibody and antigen binding fragment disclosed above.

In some embodiments, the monoclonal antibody or antigen binding targets ectodomains of TfR1.

In some embodiments, the heavy chain variable region comprises the amino acid sequence set forth as SEQ ID NO: 1. In some embodiments, the light chain variable region comprises the amino acid sequence set forth as SEQ ID NO: 2. In some embodiments, the heavy and light chain variable regions comprise the amino acid sequences set forth as SEQ ID NO: 1 and SEQ ID NO: 2, respectively.

In some embodiments, the monoclonal antibody or antigen binding fragment comprises a human framework region. In some embodiments, the monoclonal antibody is an IgG.

In some embodiments, the antigen binding fragment is a Fv, Fab, F(ab′)₂, scFV or a scFV₂ fragment.

Another embodiment of the present disclosure is an isolated nucleic acid molecule encoding the antibody or antigen binding fragment disclosed herein. In some embodiments, the isolated nucleic acid molecule encoding the antibody or antigen binding fragment comprises nucleic acid sequences set forth as SEQ ID NOs: 9 and 10. In some embodiments, the isolated nucleic acid molecule comprises nucleic acid sequences set forth as SEQ ID NOs: 11 to 16.

Another embodiment of the present disclosure is a vector comprising the nucleic acid molecule disclosed herein.

Another embodiment of the present disclosure is a host cell, comprising the nucleic acid molecule disclosed herein or a vector comprising such nucleic acid molecule.

The present disclosure further provides compositions comprising an antibody disclosed herein and kits comprising an antibody or a composition disclosed herein with instructions for the use of the antibody or the composition, respectively.

The kits may also include suitable storage containers, e.g., ampules, vials, tubes, etc., for each antibody of the present disclosure (which, e.g., may be in the form of compositions) and other reagents, e.g., buffers, balanced salt solutions, etc., for use in administering the active agents to subjects. The antibodies and/or compositions of the disclosure and other reagents may be present in the kits in any convenient form, such as, e.g., in a solution or in a powder form. The kits may further include a packaging container, optionally having one or more partitions for housing the antibodies and/or compositions and other optional reagents.

Another embodiment of the present disclosure is a method for treating or ameliorating the effects of a cancer in a subject in need thereof, comprising: a) administering to the subject a therapeutically effective amount of an agent that induces ferroptosis; b) obtaining a biological sample from the subject; c) contacting the sample with an anti-TfR1 (transferrin receptor protein 1) antibody; d) determining whether the antibody selectively binds to a cell in the sample; and e) administering a therapeutically effective amount of radiation to the subject if ferroptosis is present.

Still another embodiment of the present disclosure is a method for enhancing the anti-tumor effect of radiation in a subject with cancer undergoing radiotherapy, comprising: a) obtaining a biological sample from the subject; b) contacting the sample with an anti-TfR1 (transferrin receptor protein 1) antibody; c) determining whether the antibody selectively binds to a cell in the sample; and d) administering to the subject a therapeutically effective amount of an agent that induces ferroptosis if ferroptosis is absent.

In some embodiments, the cancer is selected from the group consisting of sarcoma, renal cell carcinoma, diffuse large B-cell lymphoma, fibrosarcoma, glioma, uterine sarcoma, primary glioblastoma, lung cancer, non-small cell lung cancer, colorectal cancer, melanoma, prostate cancer, pancreatic cancer, brain cancer, breast cancer, colon cancer, liver cancer, leiomyosarcoma, lung adenocarcinoma, and hepatocyte-derived carcinoma. In some embodiments, the cancer is resistant to radiation.

In some embodiments, the co-administration of the agent and radiation provides a synergistic effect compared to administration of either the agent or radiation alone.

In some embodiments, the anti-TfR1 antibody is selected from 3F3 anti-ferroptotic membrane antibody (3F3-FMA), anti-TfR1 3B8 2A1 antibody, anti-TfR1 H68.4 antibody, and combinations thereof.

In some embodiments, the method disclosed herein further comprises contacting the sample with at least one second antibody. In some embodiments, the at least one second antibody is selected from anti-MDA 1F83 antibody, anti-4-HNE ab46545 antibody, and combinations thereof.

In some embodiments, the agent that induces ferroptosis is selected from the group consisting of erastin, imidazole ketone erastin (IKE), piperazine erastin (PE), sulfasalazine, sorafenib, RSL3, ferroptosis inducer 56 (FIN56), caspase-independent lethal 56 (CIL56), deplete GPX4 protein, mevalonate-derived coenzyme Q₁₀, ferroptosis inducer endoperoxide (FIN02), and combinations thereof. In some embodiments, the agent that induces ferroptosis is selected from IKE, RSL3, sorafenib, and combinations thereof.

A further embodiment of the present disclosure is a composition, comprising an effective amount of the antibody or antigen binding fragment disclosed herein, or a nucleic acid molecule encoding such antibody or antigen binding fragment, and a pharmaceutically acceptable carrier.

Determining cell death mechanisms occurring in patient and animal tissues is a longstanding goal that requires suitable biomarkers and accurate quantification. However, effective methods remain elusive. To develop more powerful and unbiased analytic frameworks, the present disclosure provides a machine learning approach for automated cell death classification.

Accordingly, another embodiment of the present disclosure is a method for identifying a classifier that detects and classifies a cell death modality in a cell. The method comprises: (a) treating cultured cells with a select cell death inducer; (b) contacting the treated cells with an agent targeting a biomarker of the cell death modality; (c) carrying out an immunofluorescent assay on the cells and collecting a set of images of various cell regions; (d) analyzing the images to generate features that are statistically significantly correlated to the cell death modality; (e) performing further statistical analysis on the features generated in step (d) to select signature-features that constitute the classifier; and (f) confirming the accuracy of the classifier with a second set of images that are independently collected by repeating steps (a) to (c).

In some embodiments, the cell death modality is selected from the group consisting of apoptosis, ferroptosis, necroptosis and pyroptosis. In some embodiments, the select cell death inducer is selected from the group consisting of an apoptosis inducer, a ferroptosis inducer, a necroptosis inducer and a pyroptosis inducer.

In some embodiments, the cell death modality is apoptosis, and the select cell death inducer is an apoptosis inducer, which is staurosporine (STS).

In some embodiments, the cell death modality is ferroptosis, and the select cell death inducer is a ferroptosis inducer. Non-limiting examples of a ferroptosis inducer include erastin, imidazole ketone erastin (IKE), piperazine erastin (PE), sulfasalazine, sorafenib, RSL3, ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof.

In some embodiments, the biomarker of the cell death modality is TfR1 (transferrin receptor protein 1), and the agent targeting the biomarker is an anti-TfR1 antibody. In some embodiments, the anti-TfR1 antibody is a 3F3 anti-ferroptotic membrane antibody (3F3-FMA).

In some embodiments, DAPI and FITC-phalloidin are used in steps (b) and (c).

In some embodiments, the cell regions include nuclei, cytoplasm, and membrane.

In some embodiments, the statistical analysis performed in step (e) includes pairwise logistic regression and multinomial logistic regression.

Yet another embodiment of the present disclosure is a method for detecting a cell death modality in a system. The method comprises: (a) obtaining a biological sample from the system and contacting the biological sample with an agent targeting a biomarker of the cell death modality; (b) carrying out an immunofluorescent assay on the biological sample and collecting a set of images of various cell regions; (c) constructing a classifier following the method disclosed herein; and (d) applying the classifier on the set of images collected in step (b) to detect the cell death modality.

In some embodiments, the system is selected from cultured cells, an animal disease model, and a human subject. In some embodiments, the human subject is a patient receiving a cancer therapy. Thus, this method can be used to assess the response of cancer patients to therapy.

The following examples are provided to further illustrate the methods of the present disclosure. These examples are illustrative only and are not intended to limit the scope of the disclosure in any way.

### EXAMPLES

### Example 1

### Methods and Materials

### Generation of PE-Induced Membrane Fractions

6 L of media (1% Pen-Strep 10% FBS and 89% RPMI with L-glutamine) containing 400 million OCI-LY7 (DSMZ Cat #ACC-688, RRID:CVCL_1881) cells/L were treated with 5 μM PE (piperazine erastin). Cells were incubated for 19 h at 37° C., then production of lipid ROS was confirmed using BODIPY-C11 by flow cytometry. Ferroptotic cells were pelleted in centrifuge at 1000 rpm for 10 min at 25° C. Cells were resuspended in 1 mL lysis buffer with a pan-protease inhibitor, lysed using a dounce homogenizer. 70% cell lysis was confirmed by microscope. Pellet containing the nuclear fraction and unlysed cells was obtained by centrifuge at 700×g for 10 min at 4° C. Supernatant was spun at 700×g for 10 min at 4° C. Supernatant was then transferred to a new tube and placed in centrifuge at 10,000×g for 45 min at 4° C. Pellet consisting of total membrane was resuspended in upper phase solution. Lower phase was added and mixture was incubated on ice for 5 min. Mixture was spun at 1000×g for 5 min at 4° C. Upper phase was collected and diluted with 5× volume of H₂O and incubated on ice for 10 min. Mixture was spun at 17,000×g for 15 min at 4° C. and pellet composed of plasma membrane was collected. Fractions were confirmed using western blot.

### Purification of Monoclonal Antibodies and Generation of 3F3-FMA

Murine monoclonal antibody (clone FH3F3) was generated at the Fred Hutchinson Antibody Technology Core Facility, Seattle Wash. Briefly, female 20-week-old mice (various strains) were immunized with ferroptotic membrane fractions (see previous methods). Following a 12-week boosting protocol, splenocytes were isolated from four high titer mice and electrofused with a myeloma fusion partner generate hybridoma cells. Approximately 4,750 hybridomas positive for IgG secretion were then identified and isolated using a ClonePix2 colony picker (Molecular Devices, CPII). Primary screening of the 4,750 clones was performed by indirect flow cytometry of ferroptotic LY-7 cells (5 μM IKE treated for 19 h at 37° C., fixed with 0.01% formaldehyde in PBS for 15 min at 22° C., permeabilized in FACS buffer with 0.5% v/v Tween-20 detergent). Clones showing fluorescent staining ˜4-fold over background levels (irrelevant primary antibody plus secondary antibody) were isolated for culture and frozen down as the “Primary Clone Archive”. Clone 3F3 was further identified within the Primary Clone Archive by fluorescent staining and high-content image analysis. Clone 3F3 was then subcloned by limiting dilution-CPII colony picking. Small scale antibody productions in serum free media (Gibco Hybridoma SFM) were carried out followed by affinity chromatography (AKTA Pure, MabSelectSuRe) to obtain ˜5 mg of purified IgG1 from subclones 3F3a and 3F3 h.

### High-Content Screening and Analysis

### Automation

Plate and liquid handling was performed using a HTS platform system composed of a Sciclone G3 Liquid Handler from PerkinElmer (Waltham, Mass., USA), a MultiFIo™ Dispenser (Biotek Instruments, Bad Friedrichshall, Germany) and a Cytomat™ Incubator (Thermo Fisher Scientific, Waltham, Mass., USA) (Schorpp and Hadian, 2014). Cell seeding and assays were performed in black 384-well CellCarrier-384 Ultra Microplates (PerkinElmer, 6057300). Image acquisition and image-based quantification was performed using an Operetta®/Columbus™ high-content imaging platform (PerkinElmer, USA).

### High-Content Screening Assay

For the screening with five technical replicates, HT-1080 cells were washed with PBS, trypsinized and resuspended in cell culture medium. The cell suspension (2,000 cells in 50 μl per well) was dispensed into collagen (Sigma-Aldrich, St. Louis, Mass., USA) pre-coated 384-well plates (PerkinElmer 384-well CellCarrierUltra™). 24 h after seeding, medium was exchanged to medium containing 0.3 μM RSL3 (1 mM stock solution) dissolved in 100% dimethyl sulfoxide (DMSO) or DMSO alone. 50 μl medium with 0.3 μM RSL3/DMSO was added per well. The cells were then incubated (37° C.; 5% CO₂) for 2.5 h prior to fixation and antibody staining. After incubation time the medium was removed and cells were washed with PBS, fixed with 4% PFA for 10 min and washed again with PBS. After permeabilizing (0.5% Tween-20) for 10 min and blocking (1% BSA in PBS) for 2 h, cells were incubated with primary antibody selection in blocking solution (1:20) overnight at 4° C. The following secondary antibody was applied for 1 h at room temperature: anti-mouse Alexa488 (1:500, Invitrogen). Cells were again washed with PBS and then stained with Hoechst 33342 and Phalloidin-TRITC for 1 h at r.t. in the dark and then extensively washed with PBS afterwards. Finally, plates were recorded using the automated Operetta® microscope with the 20× high NA objective for high-resolution images (PerkinElmer, USA). For quantification, three images of each condition were recorded using three channels (Hoechst, Alexa488, TRITC). This resulted in a cell number of at least 100 cells of each condition in all wells. Quantification on cell number, cytoplasmic intensity, nucleus intensity and spot number per cell was performed using the Columbus Software (PerkinElmer, USA).

### Image Analysis

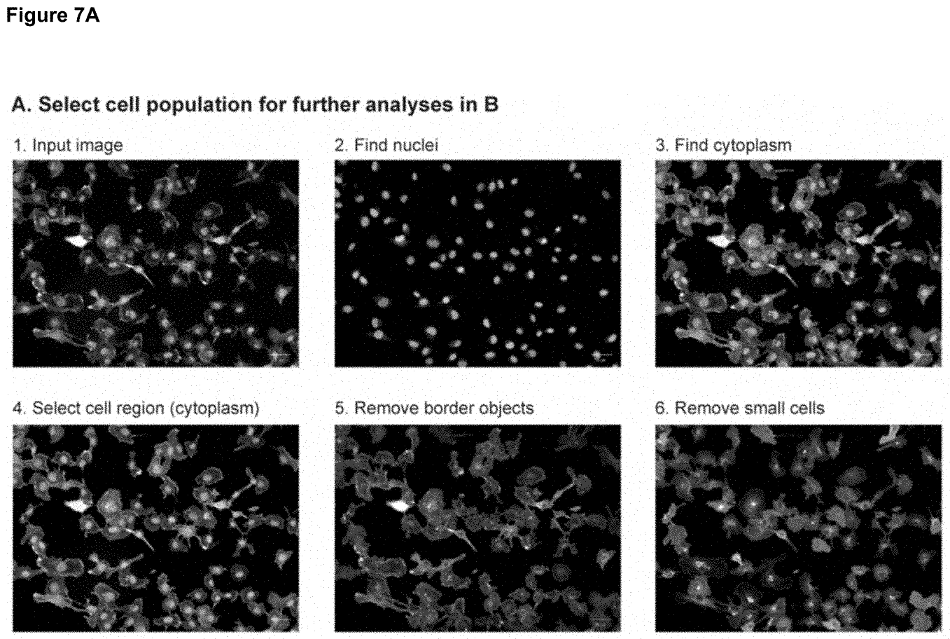

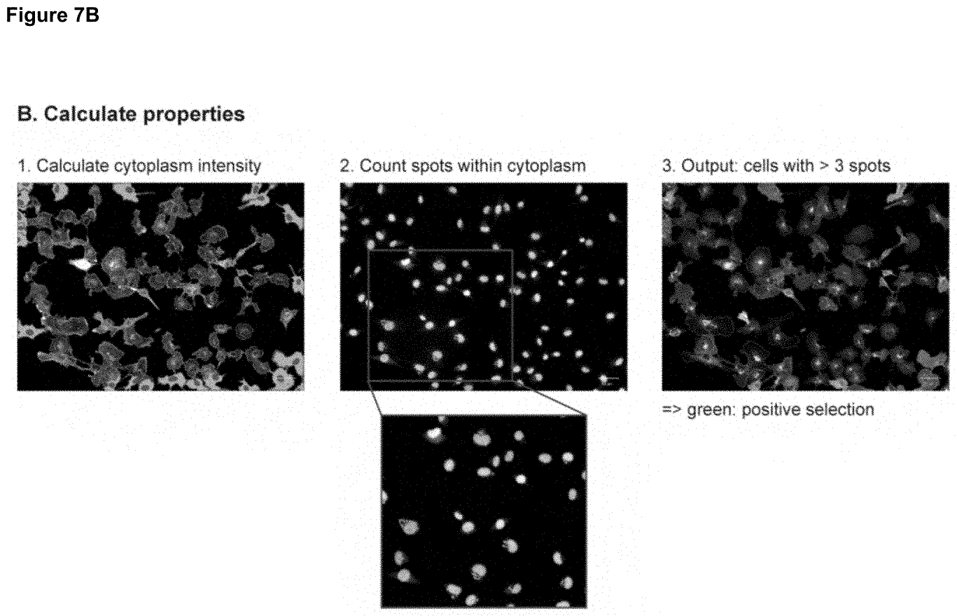

Multiparametric image analysis was performed using Columbus Software 2.8.0 (PerkinElmer). Hoechst signal was used to detect all cell nuclei. Phalloidin-TRITC signal was used to determine the cytoplasmic region to the corresponding nucleus. Moreover, we applied a filter to remove border objects (nuclei that cross image borders) and cells with extremely small nuclei (dead cells). In a next step we have calculated the morphology and Alexa488 fluorescence intensity in each cell region (nucleus and cytoplasm). In addition, we performed spot detection in the cytoplasm and used morphology and intensity parameter for each spot to define “big spots”. Each spot was detected as a small region within the corresponding image by having a higher intensity than its surrounding area. Furthermore, we selected cells with three or more “big spots” in the cytoplasm and calculated the percentage of “positive” cells in each well. Finally, a hit was defined if the ratio of cytoplasmic intensity and/or the ratio of cells with more than 3 spots was >1 in at least 3 of 5 plates after RSL3 treatment. An illustration on the automated detection method using the Hoechst-, phalloidin-TRITC- and Alexa488 antibody-signal is presented in FIGS. 7A-7B.

### Immunofluorescence (IF)

HT-1080 (ATCC Cat #CRL-7951, RRID:CVCL_0317), A-673, SK-BR-3, SK-LMS-1 and Huh-7 cells were treated with 1 μM RSL3 for 4 h, 15 μM erastin for 8 h, 10 μM IKE for 8 h, 15 μM FINO₂ for 8 h, 10 μM FIN56 for 8 h, 100 μM tBuOOH for 8 h, 1 μM staurosporine (STS) for 6 h, 2 μM camptothecin (CPT) for 24 h, 1 μM RSL3+5 μM Fer-1 for 4 h and 10 μM IKE+5 μM Fer-1 for 8 h on poly-lysine-coated cover slips (Sigma Aldrich P4832) in 24-well plate. Media were taken out and the cells were gently washed with PBS++(PBS with 1 mM CaCl₂) and 0.5 mM MgCl₂) twice. The cells were fixed and permeabilized by adding 200 μL/well of 4% paraformaldehyde (PFA) in PBS with 0.1% Triton X-100 (PBT), and incubated at room temperature for 18 min. The cells were then washed with PBT three times. Then the cells were blocked with 5% goat serum (ThermoFisher 50197Z) in PBT for 1 h at room temperature. The cells were incubated with purified mouse monoclonal antibodies (1:5 dilution), mouse mAb 3F3 FMA (1:500 dilution), Transferrin Receptor/CD71 Monoclonal Antibody, Clone: H68.4, Invitrogen (Thermo Fisher Scientific Cat #13-6800, RRID:AB_2533029, 1:250 dilution), Cd71 (D7G9X) XP® Rabbit mAb (Cell Signaling Technology Cat #13113, RRID:AB_2715594, 1:100 dilution), CD71 (3B8 2A1) (Santa Cruz Biotechnology Cat #sc-32272, RRID:AB_627167, 1:50 dilution), Tom20 (FL-145) (Santa Cruz Biotechnology Cat #sc-11415, RRID:AB_2207533, 1:250 dilution), PDI antibody [RL90]-ER Marker (Abcam Cat #ab2792, RRID:AB_303304, 1:100 dilution), Gm130 (D6B1) XP® Rabbit mAb (Cell Signaling Technology Cat #12480, RRID:AB_2797933, 1:3200 dilution), Anti-Malondialdehyde antibody (Abcam Cat #ab6463, RRID:AB_305484, 1:400 dilution), Anti-4 Hydroxynonenal antibody (Abcam Cat #ab46544, RRID:AB_722493, 1:50 dilution), ACSL4 Antibody (F-4) (Santa Cruz Biotechnology Cat #sc-365230, RRID:AB_10843105, 1:50 dilution), mouse mAb 1F83 (1:100 dilution), which specifically recognizes the malondialdehyde (MDA)-lysine adduct 4-methyl-1,4-dihydropyridine-3,5-dicarbaldehyde (MDHDC) (Yamada et al., 2001), in PBT with 1% BSA and 5% goat serum overnight at 4° C. The cells were washed with PBT for 5 min three times. The cells were incubated with Goat anti-Mouse IgG (H+L) Highly Cross-Adsorbed Secondary Antibody, Alexa Fluor 594 (Thermo Fisher Scientific Cat #A-11032, RRID:AB_2534091, 1:200 dilution) or Goat anti-Rabbit IgG (H+L) Highly Cross-Adsorbed Secondary Antibody, Alexa Fluor 488 (Thermo Fisher Scientific Cat #A-11034, RRID:AB_2576217, 1:200 dilution) at room temperature for 1 h. The cells were washed with PBT for 5 min three times. ProLong Diamond antifade mountant with DAPI (ThermoFisher P36962) was added to stain the nucleus. All images were captured on a Zeiss LSM 800 confocal microscope at Plan-Apochromat 63×/1.40 Oil DIC objective with constant laser intensity for all samples. When applicable, line-scan analysis was performed on representative confocal microscopy images using Zeiss LSM software to qualitatively visualize fluorescence overlap.

### Quantification Method

The quantification of the intensity of antibodies was analyzed using CellProfiler 3.1.8 (Carpenter et al., 2006) (CellProfiler Image Analysis Software, RRID:SCR_007358). Nuclei were firstly identified as primary objects using global minimum cross entropy strategy. Cytoplasm were then identified as secondary objects based on primary objects by propagation using global minimum cross entropy strategy. The plasma membranes were identified as the outermost 5 pixels of cytoplasm. Then mean intensities and size areas of nuclei, cytoplasm and plasma membranes were then measured and reported. Graphs were drawn by Prism 7.

### Immunoprecipitation-Mass Spectrometry (IP-MS)

HT-1080 cells were seeded in DMEM (Corning 10-013-CM) and 10% Hi-FBS with 1% penicillin and streptomycin (PS) with 1% MEM Non-Essential Amino Acids Solution (100×) (Thermo Fisher Scientific 11140-076) 16 h prior to use. DMSO or 1 μM of RSL3 was added and incubated for 4 h. Following treatment, the medium was aspirated from each dish and cells were washed twice with PBS. Cells were lysed with 70 μl lysis buffer (RIPA buffer from ThermoFisher, cat. 89900, 1 mM EDTA, 1 mM PMSF, 1× Halt™ protease inhibitor cocktail from ThermoFisher, cat. 78430 and 1× Halt™ phosphatase inhibitor cocktail from ThermoFisher, cat. 78426). Unlysed cells and debris were pelleted for 15 min at 16,000×g at 4° C. The samples were incubated with 10 μg of 3F3 FMA overnight at 4° C. with shaking. The next day, Thermo Scientific Pierce Protein AG Magnetic Beads (Thermo Fisher Scientific 88802) were washed with TBS with 0.05% Tween 20 (wash buffer) and then were incubated with sample/antibody mixture for 1 h with mixing. The beads were collected with a magnetic stand and then washed with wash buffer for three times. The beads were used for mass spectrometry.

Trypsin digestion was performed overnight at 37° C. Supernatants were collected and dried down in a speed-vac, and peptides were dissolved in a solution containing 3% acetonitrile and 0.1% formic acid. Peptides were desalted with C18 disk-packed stage-tips. Desalted peptides were injected onto an EASY-Spray PepMap RSLC C18 50 cm×75 μm column (Thermo Scientific), which was coupled to the Orbitrap Fusion Tribrid mass spectrometer (Thermo Scientific). Peptides were eluted with a non-linear 110 min gradient of 5-30% buffer B (0.1% (v/v) formic acid, 100% acetonitrile) at a flow rate of 250 nl/min. The column temperature was maintained at a constant 50° C. during all experiments. Thermo Scientific™ Orbitrap Fusion™ Tribrid™ mass spectrometer was used for peptide MS/MS analysis. Survey scans of peptide precursors were performed from 400 to 1575 m/z at 120K FWHM resolution (at 200 m/z) with a 2×10⁵ ion count target and a maximum injection time of 50 ms. The instrument was set to run in top speed mode with 3 s cycles for the survey and the MS/MS scans. After a survey scan, tandem MS was performed on the most abundant precursors exhibiting a charge state from 2 to 6 of greater than 5×10³ intensity by isolating them in the quadrupole at 1.6 Th. CID fragmentation was applied with 35% collision energy and resulting fragments were detected using the rapid scan rate in the ion trap. The AGC target for MS/MS was set to 1×10⁴ and the maximum injection time limited to 35 ms. The dynamic exclusion was set to 45 s with a 10 ppm mass tolerance around the precursor and its isotopes. Monoisotopic precursor selection was enabled. MS data analysis

Raw mass spectrometric data were analyzed using MaxQuant v. 1.6.1.0 (Cox and Mann, 2008) (MaxQuant, RRID:SCR_014485) and employed Andromeda for database search (Cox et al., 2011) at default settings with a few modifications. The default was used for first search tolerance and main search tolerance: 20 ppm and 6 ppm, respectively. MaxQuant was set up to search the reference Human proteome database downloaded from Uniprot. MaxQuant performed the search trypsin digestion with up to 2 missed cleavages. Peptide, Site and Protein FDR were all set to 1% with a minimum of 1 peptide needed for Identification but 2 peptides needed to calculate a protein level ratio. The following modifications were used as fixed carbamidomethyl modification of cysteine, and oxidation of methionine (M), Deamination for asparagine or glutamine (NQ) and acetylation on N-terminal of protein were used as variable modifications. MaxQuant combined folder was uploaded in scaffold for data visualization.

siRNA Knockdown Assay

10 μM of siRNAs were combined with 250 μl of Opti-MEM serum-free media (Life Technologies 31985-070) in one tube. 6 μl of Lipofectamine RNAiMAX (Thermo Fisher Scientific 13778150) was combined with 250 μl of Opti-Mem media in another tube. They were equilibrated at r.t. for 5 min. Then two tubes were combined, transferred into 6-well plate and incubated at 37° C. for 20 min. 0.25 million HT-1080 cells were then added to 6-well plate and incubated for 48 h. Cells were reseeded in regular media for additional 24 h. Regular IF procedure was then conducted.

### Flow Cytometry and Analysis

Cells were resuspended in 500 mL HBSS containing 2 μM C11-BODIPY (BODIPY 581/591 C11) (Thermo Fisher Scientific, D3861) and incubated at 37° C. for 15 min. Cells were pelleted and resuspended in HBSS strained through a 35 μm cell strainer (Fisher Scientific 08-771-23). Fluorescence intensity was measured on the FL1 channel with gating to record live cells only (gate constructed from DMSO treatment group). A minimum of 10,000 cells were analyzed per condition. Analysis was performed using FlowJo software.

HT-1080 cells were treated with DMSO or 1 μM RSL3 for 4 h. The cells were harvested by 0.25% Trypsin-EDTA (1×) (Invitrogen 25200-114) and washed with HBSS once. The cells were resuspended in 5% goat serum (ThermoFisher 50197Z) for 30 min on ice. The cells were incubated with mAb 3F3 FMA (1:500 dilution), Transferrin Receptor/CD71 Monoclonal Antibody, Clone: H68.4, Invitrogen (Thermo Fisher Scientific Cat #13-6800, RRID:AB_2533029, 1:250 dilution), CD71 (3B8 2A1) (Santa Cruz Biotechnology Cat #sc-32272, RRID:AB_627167, 1:50 dilution), Anti-Malondialdehyde antibody (Abcam Cat #ab6463, RRID:AB_305484, 1:400 dilution), Anti-4 Hydroxynonenal antibody (Abcam Cat #ab46544, RRID:AB_722493, 1:50 dilution), and mouse mAb 1F83 (1:100 dilution), which specifically recognizes the malondialdehyde (MDA)-lysine adduct 4-methyl-1,4-dihydropyridine-3,5-dicarbaldehyde (MDHDC) (Yamada et al., 2001) for 1 h on ice. The cells were washed with HBSS for 5 min three times by centrifugation. The cells were incubated with Goat anti-Mouse IgG (H+L) Secondary Antibody, Alexa Fluor® 488 conjugate (Thermo Fisher Scientific Cat #A-11001, RRID:AB_2534069, 1:200 dilution) or Goat anti-Rabbit IgG (H+L) Highly Cross-Adsorbed Secondary Antibody, Alexa Fluor 488 (Thermo Fisher Scientific Cat #A-11034, RRID:AB_2576217, 1:200 dilution) for 30 min on ice. The cells were washed with HBSS twice by centrifugation, then resuspended in HBSS strained through a 35 μm cell strainer (Fisher Scientific 08-771-23). Fluorescence intensity was measured on the FL1 channel with gating to record live cells only (gate constructed from DMSO treatment group). A minimum of 10,000 cells were analyzed per condition. Analysis was performed using FlowJo software (FlowJo, RRID:SCR_008520).

### Immunohistochemistry (IHC)

Tumor tissues were fixed in 4% paraformaldehyde (PFA) for 24 h at 4° C. followed by washing with PBS three times. The tissues were perfused in 30% sucrose for 24 h at 4° C. for cryo-protection. The samples were embedded in OCT cryostat sectioning medium, and then moved directly into a cryostat. After equilibration of temperature, frozen tumor tissues were cut into 5 μm thick sections. Tissue sections were mounted on to poly-L-lysine coated slides by placing the cold sections onto warm slides. Slides were stored at −80° C. until staining. For staining, slides were warmed to room temperature followed by washing with PBS twice. A hydrophobic barrier pen was used to draw a circle on each slide. The slides were permeabilized with PBS with 0.4% Triton X-100 (PBT) twice before non-specific-binding blocking by incubating the sections with 10% goat serum (ThermoFisher 50197Z) for 30 min at room temperature. The sections were separately incubated with mouse mAb 3F3 FMA (1:500 dilution), Transferrin Receptor/CD71 Monoclonal Antibody, Clone: H68.4, Invitrogen (Thermo Fisher Scientific Cat #13-6800, RRID:AB_2533029, 1:250 dilution), CD71 (3B8 2A1) (Santa Cruz Biotechnology Cat #sc-32272, RRID:AB_627167, 1:50 dilution), Anti-Malondialdehyde antibody (Abcam Cat #ab6463, RRID:AB_305484, 1:400 dilution), Anti-4 Hydroxynonenal antibody (Abcam Cat #ab46544, RRID:AB_722493, 1:50 dilution), and mouse mAb 1F83 (1:100 dilution), which specifically recognizes the malondialdehyde (MDA)-lysine adduct 4-methyl-1,4-dihydropyridine-3,5-dicarbaldehyde (MDHDC) (Yamada et al., 2001) overnight at 4° C. in humidified chambers. Sections were washed with PBT for three times before incubating with Goat anti-Mouse IgG (H+L) Highly Cross-Adsorbed Secondary Antibody, Alexa Fluor 594 (Thermo Fisher Scientific Cat #A-11032, RRID:AB_2534091, 1:1000 dilution) or Goat anti-Rabbit IgG (H+L) Highly Cross-Adsorbed Secondary Antibody, Alexa Fluor 488 (Thermo Fisher Scientific Cat #A-11034, RRID:AB_2576217, 1:1000 dilution) at room temperature for 1 h. Slides were then washed twice with PBT three times. ProLong Diamond antifade mountant with DAPI (ThermoFisher P36962) was added onto slides, which were then covered with the coverslips, sealed by clear fingernail polish and observed under confocal microscopy. All images were captured on a Zeiss LSM 800 confocal microscope at Plan-Apochromat 63×/1.40 Oil DIC objective with constant laser intensity for all analyzed samples. The intensity above threshold of the fluorescent signal of the bound antibodies was analyzed using NIH ImageJ software (ImageJ, RRID:SCR_003070). Data were expressed as fold change comparing with the vehicle.

### Western Blot

HT-1080 cells were seeded in DMEM (Corning 10-013-CM) and 10% Hi-FBS with 1% penicillin and streptomycin (PS) with 1% MEM Non-Essential Amino Acids Solution (100×) (Thermo Fisher Scientific 11140-076) 16 h prior to use. DMSO or 1 μM of RSL3 was added and incubated for 4 h. Following treatment, the medium was aspirated from each dish and cells were washed twice with PBS. Cells were lysed with 70 μl lysis buffer (RIPA buffer from ThermoFisher, cat. 89900, 1 mM EDTA, 1 mM PMSF, 1× Halt™ protease inhibitor cocktail from ThermoFisher, cat. 78430 and 1× Halt™ phosphatase inhibitor cocktail from ThermoFisher, cat. 78426). Unlysed cells and debris were pelleted for 15 min at 16,000×g at 4° C. Samples were separated using SDS-PAGE and transferred to a polyvinylidene difluoride membrane. Transfer was performed using the iBlot2 system (Invitrogen). Membranes were treated with Li-COR Odyssey blocking buffer for at least 1 h at r.t., then incubated with mouse mAb 3F3 FMA (1:500 dilution), Transferrin Receptor/CD71 Monoclonal Antibody, Clone: H68.4, Invitrogen (Thermo Fisher Scientific Cat #13-6800, RRID:AB_2533029, 1:250 dilution), Cd71 (D7G9×) XP® Rabbit mAb (Cell Signaling Technology Cat #13113, RRID:AB_2715594, 1:100 dilution), CD71 (3B8 2A1) (Santa Cruz Biotechnology Cat #sc-32272, RRID:AB_627167, 1:50 dilution) in a 1:1 solution of PBS-T (PBS with 0.1% Tween 20) and Li-COR odyssey blocking buffer overnight at 4° C. Following three 5 min washes in PBS-T, the membrane was incubated with secondary antibodies, goat anti-rabbit or goat anti-mouse IgG antibody conjugated to an IRdye at 800CW (LI-COR Biosciences Cat #926-32211, RRID:AB_621843, 1:3000 dilution) and Alexa Fluor 680 goat anti-mouse IgG (H+L) (Thermo Fisher Scientific Cat #A-21058, RRID:AB_2535724, 1:3000 dilution) in a 1:1 solution of PBS-T and Li-COR Odyssey blocking buffer for 1 h at r.t. Following three 5 min washes in PBS-T, the membrane was scanned using the Li-COR Odyssey Imaging System.

qPCR

HT-1080 cells were seeded in 6-well plates at a density of 400 k cells/well and incubated overnight. The following day, IKE or RSL3 were diluted into wells from stock solutions and treated for indicated time periods. Following treatment, cells were rinsed in cold PBS, trypsinized, and pelleted in Eppendorf tubes. RNA was isolated from cell pellets using Qiagen's RNeasy extraction kit, following manufacturer's instructions (Qiagen). RNA quantity and quality was evaluated by a nanodrop spectrophotometer (Thermo Fisher Scientific). cDNA was generated from 2 μg of total RNA, which was then diluted ten-fold and used as a template in qPCR reactions on a Viia7 Real-Time system. Gene specific primers were used as follows: TfR1 FW: 5′ ACCATTGTCATATACCCGGTTCA 3′ (SEQ ID No: 18); TFR1 RV: 5′ CAATAGCCCAAGTAGCCAATCAT 3′ (SEQ ID No: 19); GAPDH FW: 5′ CTCCAAAATCAAGTGGGGCG 3′ (SEQ ID No: 20); GAPDH RV: 5′ ATGACGAACATGGGGGCATC 3′ (SEQ ID No: 21).

### Animal Models

### B Cell Lymphoma Mouse Xenograft Model

B cell lymphoma mouse xenograft model was generated by injecting 6-week-old NCG mice with 10 million SU-DHL-6 cells subcutaneously. The mice were treated after the tumor size reached 100 mm³. Mice were separated randomly into treatment groups of 3 and dosed with vehicle and 40 mg/kg IKE once daily by IP for 14 days. 3 h after the final dosage, mice were euthanized with CO₂, and tumors were dissected, frozen on dry ice, and stored at −80° C. All experiments using animals were performed according to protocols approved by the Institutional Animal Care and Use Committee (IACUC) at Columbia University, NY, USA.

### Hepatocellular Carcinoma (HCC) Mouse Xenograft Model

Hepatocellular carcinoma (HCC) mouse xenograft tissue samples were generated by injecting 6-week-old NCG mice (2 male and 2 female per group) with 5 million human Huh-7 HCC cells subcutaneously. After three weeks, mice were dosed with vehicle or 50 mg/kg IKE once daily by IP for 2 days. 3 h after the final dosage, mice were euthanized with CO₂. Tumors and liver tissues were dissected, frozen on dry ice, and stored at −80° C. All experiments using animals were performed according to protocols approved by the Institutional Animal Care and Use Committee (IACUC) at Columbia University, NY, USA.

### Murine Glioma Model

All procedures were performed according to the Columbia University Medical Center Institutional Animal Care and Use Committee. Murine glioma cell lines were created from tumor bearing mice. These tumors were generated by injection of a PDGF-IRES-Cre retrovirus into the subcortical white matter of mice with floxed PTEN and/or p53 (Sonabend et al., 2013). After mice reached end stage, the tumors were dissociated and primary cell cultures were created. These cells harbored the specific mutations of the original tumors, and could be re-injected to form gliomas in c57/B6 mice with high fidelity. Briefly, mice between the ages of 6-8 weeks received 50,000 murine glioma cells through stereotactic injection after being anesthetized with a ketamine/xylazine cocktail (87.5 mg/12.5 mg w/w). After cessation of toe-pinch reflex, the scalp was shaved and cleaned with serial use of betadine and 70% ethanol swabs. An incision was made and the skull was exposed. A burr hole was created, 2 mm anterior, 2 mm lateral and 2 mm deep to the right of the bregma. The cells were injected into the subcortical white matter over a period of 3 minutes (0.333 μL/min). Once the injection ceased, the needle was left in place for 1 minute before being slowly withdrawn. After tumor developed, brains were harvested and fixed in 4% paraformaldehyde and embedded in paraffin. 5 micron sections were made for staining.

KEY RESOURCES TABLE

| | | |
|---|---|---|
| REAGENT or RESOURCE | SOURCE | IDENTIFIER |

Antibodies

| | | |
|---|---|---|
| 3F3-FMA | This disclosure | N/A |
| Anti-dihydropyridine-MDA-lysine | Reference (Yamada et | N/A |
| adduct | al., 2001) | |
| mouse mAb 1F83 | | |
| Transferrin Receptor/CD71 | Thermo Fisher | Cat#13-6800, |
| Monoclonal Antibody, Clone: H68.4, | Scientific | RRID: AB_2533029 |
| Invitrogen | | |
| Cd71 (D7G9X) XP ® Rabbit mAb | Cell Signaling | Cat#13113, |
| | Technology | RRID: AB 2715594 |
| CD71 (3B8 2A1) | Santa Cruz | Cat#sc-32272, |
| | Biotechnology | RRID: AB 627167 |
| Tom20 (FL-145) | Santa Cruz | Cat#sc-11415, |
| | Biotechnology | RRID: AB 2207533 |
| PDI antibody [RL90]-ER Marker | Abeam | Cat#ab2792, |
| | | RRID: AB 303304 |
| Gm 130 (D6B1) XP ® Rabbit mAb | Cell Signaling | Cat#12480, |
| | Technology | RRID: AB 2797933 |
| Anti-Malondialdehyde antibody | Abeam | Cat#ab6463, |
| | | RRID: AB 305484 |
| Anti-4 Hydroxynonenal antibody | Abeam | Cat#ab46544, |
| | | RRID: AB_722493 |
| ACSL4 Antibody (F-4) | Santa Cruz | Cat# sc-365230, |
| | Biotechnology | RRID: AB_10843105 |
| Goat anti-Mouse IgG (H + L) | Thermo Fisher | Cat#A-11032, |
| Highly Cross-Adsorbed Secondary | Scientific | RRID: AB_2534091 |
| Antibody, Alexa Fluor 594 | | |
| Goat anti-Rabbit IgG (H + L) | Thermo Fisher | Cat#A-11034, |
| Highly Cross-Adsorbed Secondary | Scientific | RRID: AB_2576217 |
| Antibody, Alexa Fluor 488 | | |
| EGFR Monoclonal Antibody (H11) | Thermo Fisher | Cat# MA5-13070, |
| | Scientific | RRID: AB_10977527 |
| CD20 | Abeam | Cat#ab194970 |
| Glypican 3 Polyclonal Antibody | Thermo Fisher | Cat# PA5-47256, |
| | Scientific | RRID: AB 2608607 |
| CD8a (D8A8Y) Rabbit mAb antibody | Cell Signaling | Cat# 85336, |
| | Technology | RRID: AB_2800052 |
| CD45 (D9M8I) XP antibody | Cell Signaling | Cat# 13917, |
| | Technology | RRID: AB_2750898 |
| Cleaved Caspase-3 (Asp175) | Cell Signaling | Cat# 9661, |
| Antibody | Technology | RRID: AB_2341188 |
| Cleaved PARP (Asp214) (D64E10) | Cell Signaling | Cat# 5625, |
| XP ® Rabbit mAb | Technology | RRID: AB_10699459 |

Biological Samples

| | | |
|---|---|---|
| B cell lymphoma mouse xenograft | Reference(Zhang et al., | N/A |
| model | 2019) | |
| Hepatocellular carcinoma (HCC) | Brent R. Stockwell lab | N/A |
| mouse xenograft model | | |
| Murine glioma model | Peter D. Canoll lab | N/A |

Chemicals, Peptides, and Recombinant Proteins

| | | |
|---|---|---|
| RSL3 | Reference (Yang and | N/A |
| | Stockwell, 2008) | |
| Imidazole ketone erastin (IKE) | Reference (Larraufie et | N/A |
| | al., 2015)) | |
| Erastin | Reference (Dolma et | N/A |
| | al., 2003) | |
| FIN56 | Reference (Shimada et | N/A |
| | al., 2016) | |
| FINO₂ | Reference(Gaschler et | N/A |
| | al., 2018) | |
| Staurosporine (STS) | Selleck Chemicals | Cat#S1421 |
| Ferrostatin-1 (Fer-1) | Reference(Skouta et | N/A |
| | al., 2014) | |
| C11-BODIPY (BODIPY 581/591 C11) | Thermo Fisher | Cat#D3861 |
| | Scientific | |

Critical Commercial Assays

| | | |
|---|---|---|
| Plasma Membrane Protein Extraction | Abeam | Cat#ab65400 |
| Kit | | |

Experimental Models: Cell Lines

| | | |
|---|---|---|
| OCI-LY7 | DSMZ | Cat# ACC-688, |
| | | RRID: CVCL 1881 |
| HT-1080 | ATCC | Cat# CRL-7951, |
| | | RRID: CVCL 0317 |
| A-673 | Stockwell lab | N/A |
| SK-BR-3 | Stockwell lab | N/A |
| SK-LMS-1 | Stockwell lab | N/A |
| Huh-7 | Stockwell lab | N/A |

Oligonucleotides

| | | |
|---|---|---|
| SMARTpool: ON-TARGETplus TFRC | Dharmacon | Cat# L-003941-00-0005 5 nmol |
| siRNA | | |
| qPCR primers targeting TfR1 FWD: | This disclosure | N/A |
| 5′ ACCATTGTCATATACCCGGTTCA 3′ | | |
| (SEQ ID No: 18); TFR1 RV: | | |
| 5′ CAATAGCCCAAGTAGCCAATCAT 3′ | | |
| (SEQ ID No: 19); GAPDH FW: | | |
| 5′ CTCCAAAATCAAGTGGGGCG 3′ | | |
| (SEQ ID No: 20); GAPDH RV: | | |
| 5′ ATGACGAACATGGGGGCATC 3′ | | |
| (SEQ ID No: 21). | | |

Software and Algorithms

| | | |
|---|---|---|
| Columbus Software 2.8.0 | PerkinElmer | https://www.perkinelmer.com |
| CellProfiler 3.1.8 | CellProfiler Image | https://cellprofiler.org |
| | Analysis Software | |
| Prism, Version 7.0 | GraphPad Software | https://www.graphpad.com/scientific- |
| | | software/prism/ |
| MaxQuant v.1.6.1.0 | MaxQuant | https://www.maxquant.orq/ |

Other

| | | |
|---|---|---|
| Prolong Diamond antifade mountant | ThermoFisher | Cat#P36962 |
| with DAPI | | |
| RIPA buffer | Thomas Scientific | Cat#89900 |
| 10% goat serum | Thermo Fisher | Cat#50197Z |
| | Scientific | |

### Example 2

### Screen of 672 Monoclonal Antibodies Generated by Infecting Mice with Piperazine Erastin (PE)-Induced Membrane Fractions

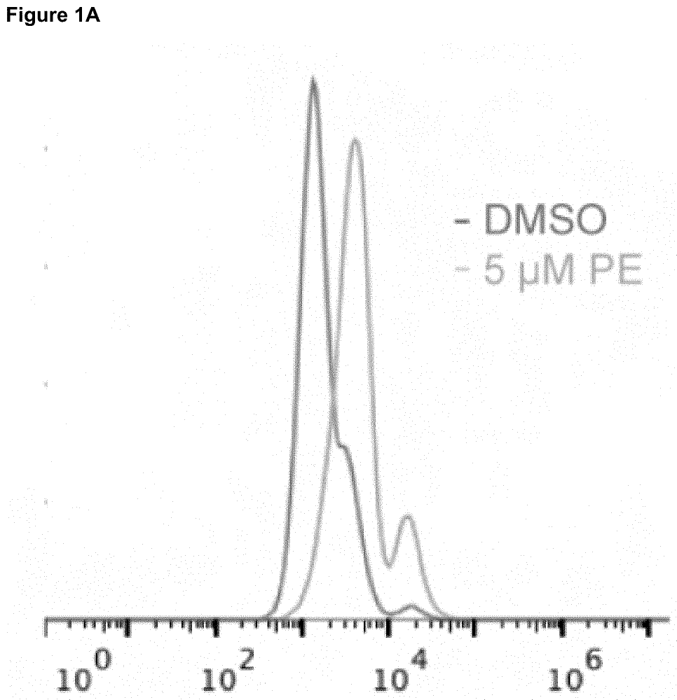

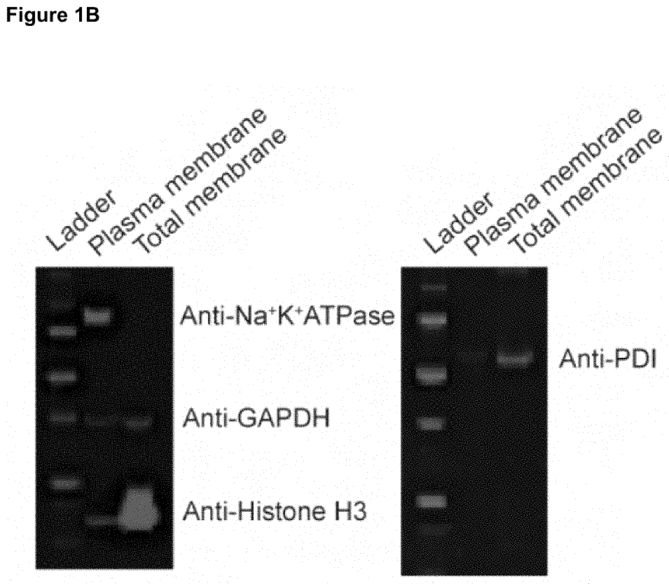

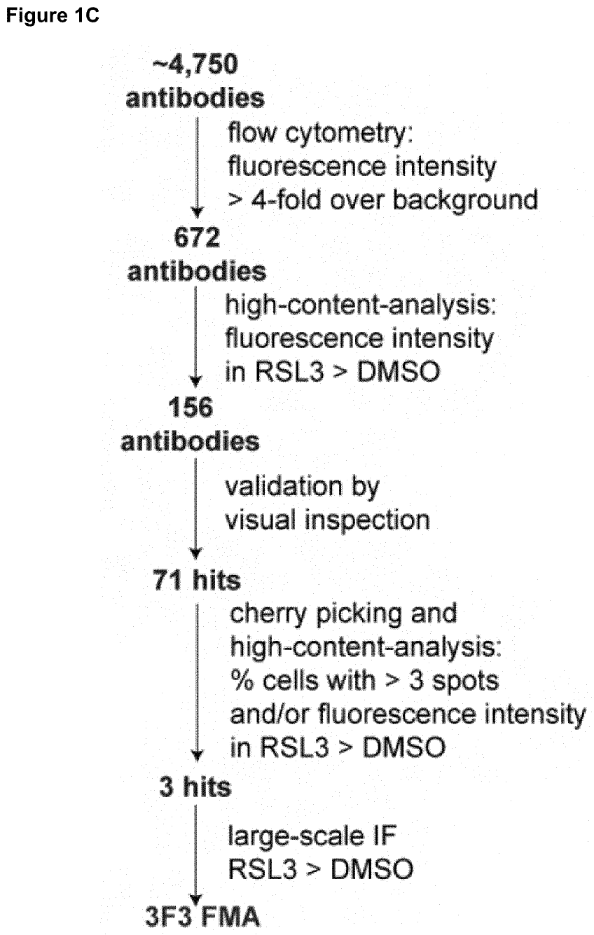

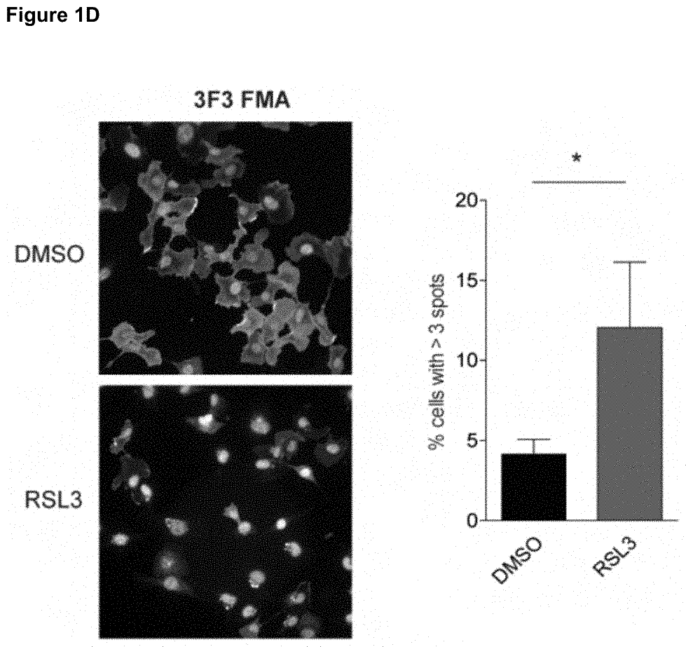

To identify a reliable and specific ferroptosis marker, we started with generation of an untargeted pool of monoclonal antibodies, by injecting mice with ferroptotic membrane fractions. Suspension OCI-LY7 cells (DLBCL, diffuse large B cell lymphoma cells) were incubated with PE, a class 1 ferroptosis inducer, for 19 h at 37° C. An increase in lipid peroxides, confirmed by fluorescent changes of C11-BODIPY, was used as a sign of ferroptosis to select this time point and concentration (FIG. 1A). Ferroptotic cells were then lysed, homogenized, and centrifuged to obtain purified total membrane and plasma membrane fractions (see materials and methods). The purified total membrane and plasma membrane were confirmed using organelle markers by western blot (FIG. 1B).

Female 20-week-old mice were immunized with ferroptotic membrane fractions. Following a 12-week boosting protocol, splenocytes were isolated and electrofused with a myeloma fusion partner to generate hybridoma cells (Alkan, 2004). ˜4,750 antibodies to unknown targets were purified and tested in a high-throughput screen (FIG. 1C). 672 antibodies showed increased intensities in IKE-treated cells by flow cytometry, and then 156 antibodies showed increased fluorescence intensity in RSL3-treated cells by high-content analysis. 71 positives were selected through visual inspection to remove false positives. Three hits stood out by picking cells with more than three bright spots in the cytoplasm or overall higher cytoplasmic intensity over five replicates. Image analysis of 3F3-FMA is provided as an example (FIG. 1D and FIGS. 7A-7B).

### Example 3

### 3F3-FMA is Identified and Validated as Ferroptosis-Detecting Antibody

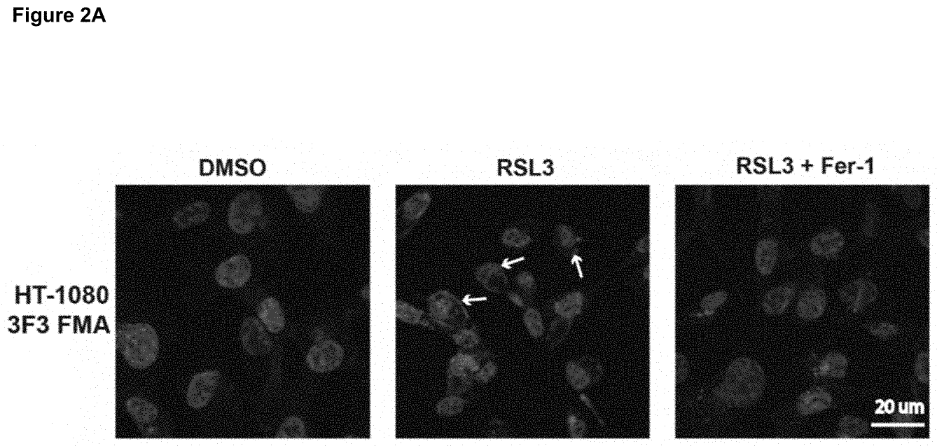

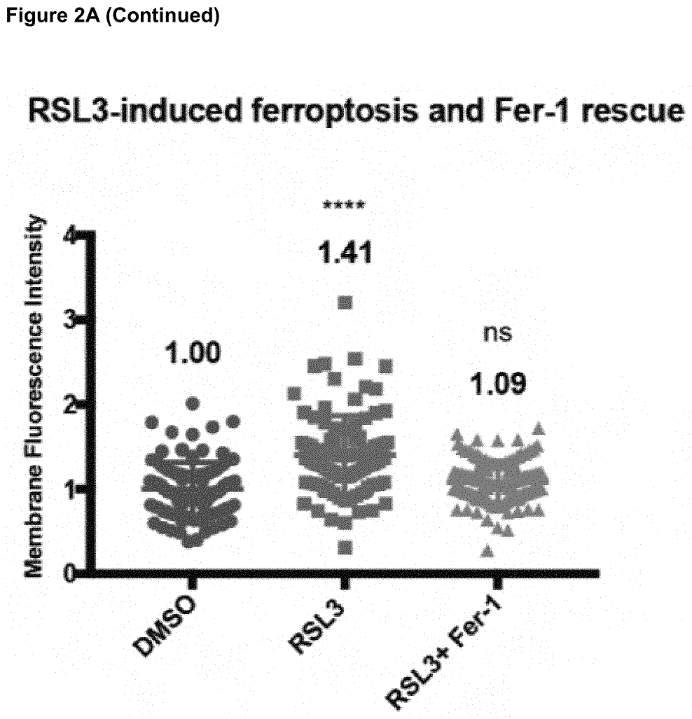

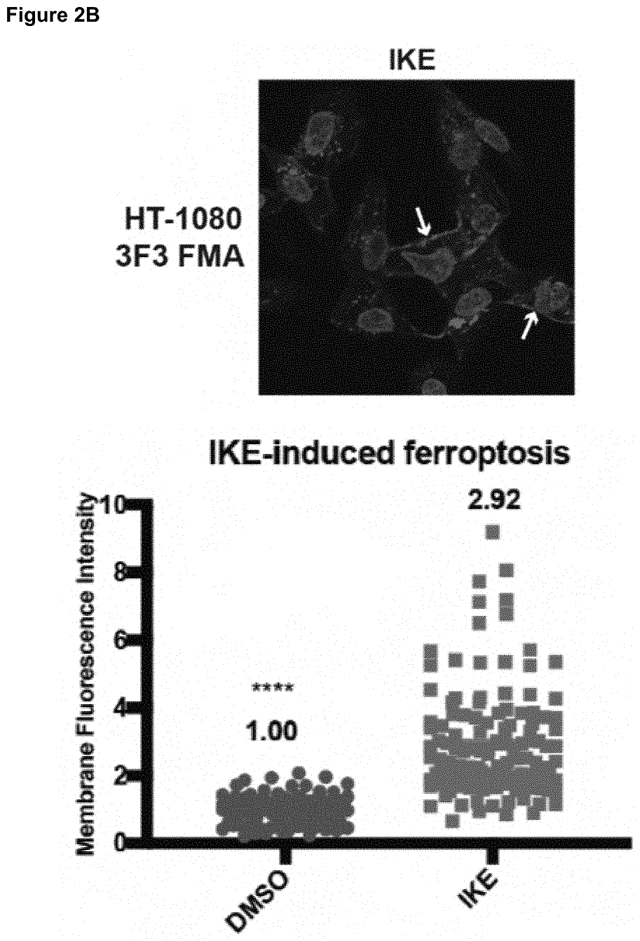

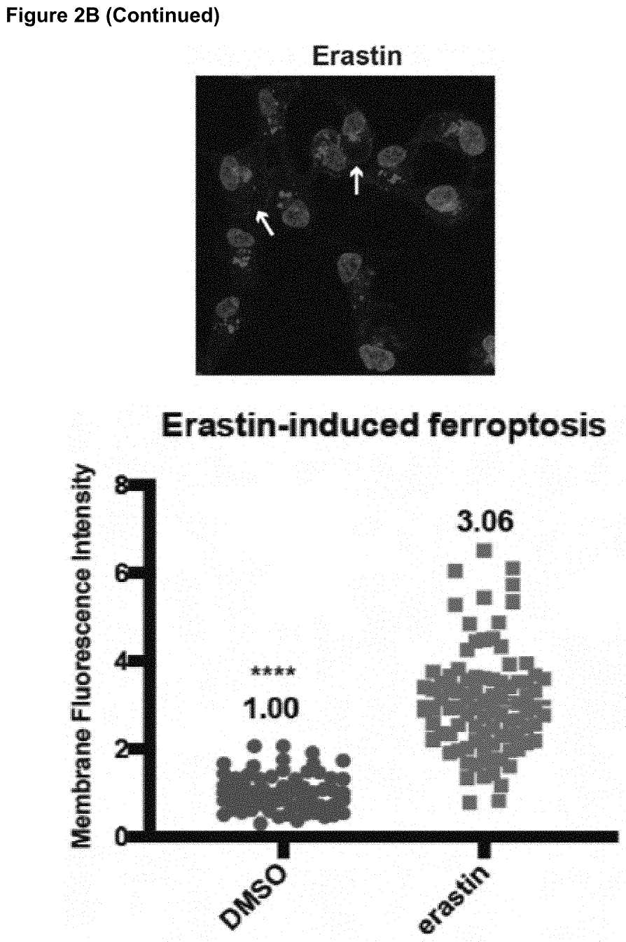

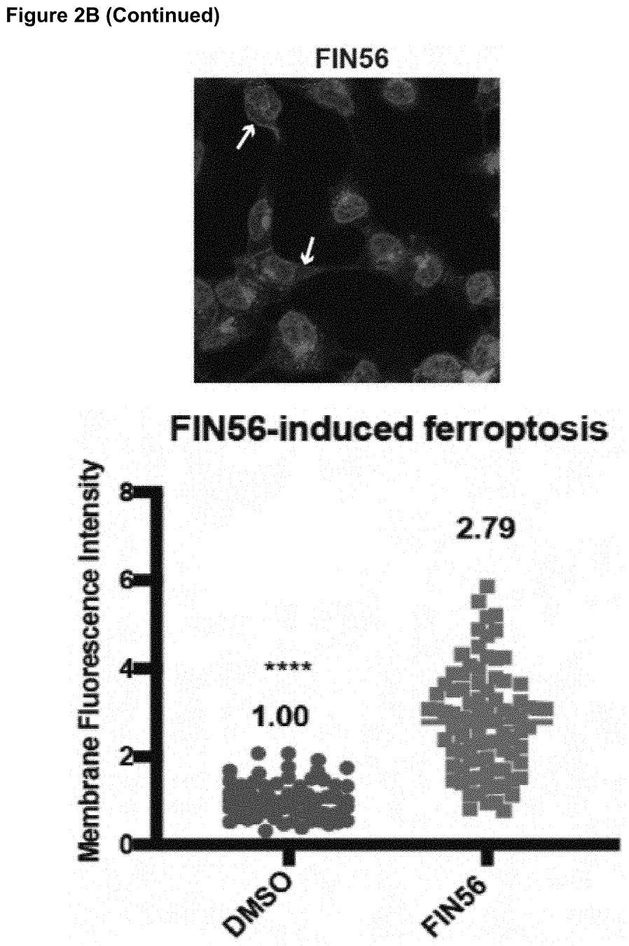

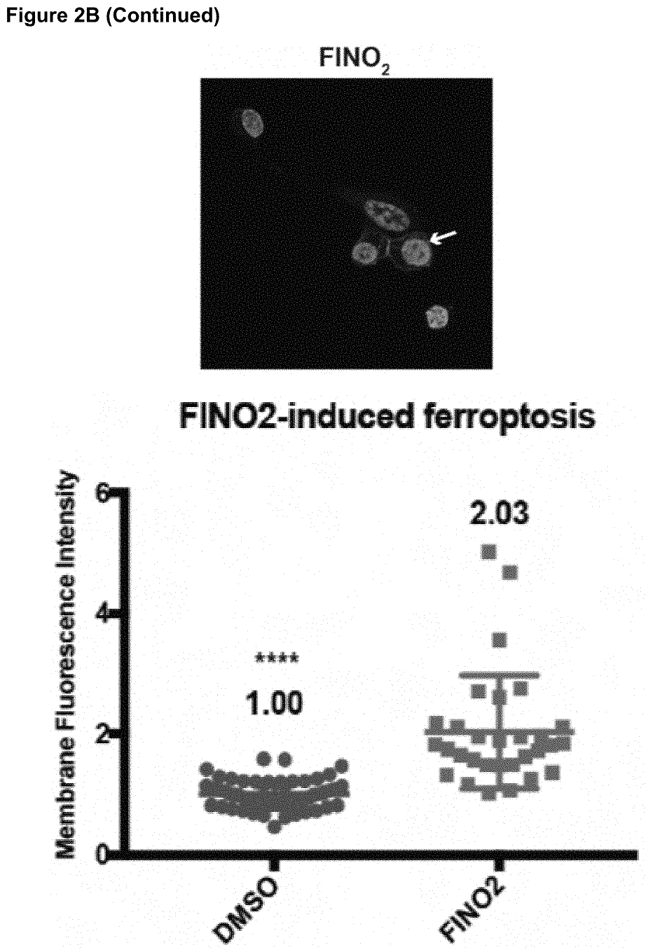

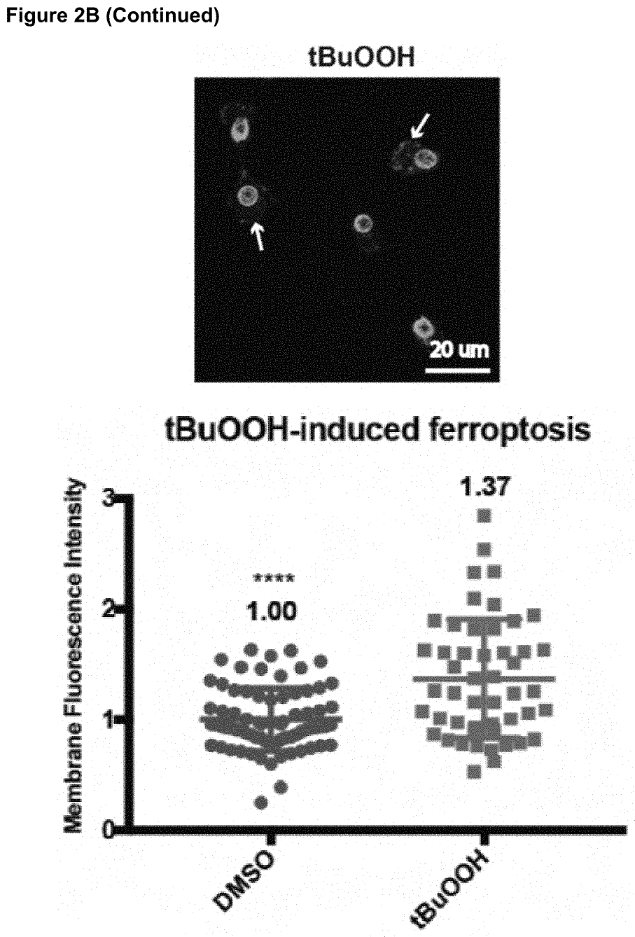

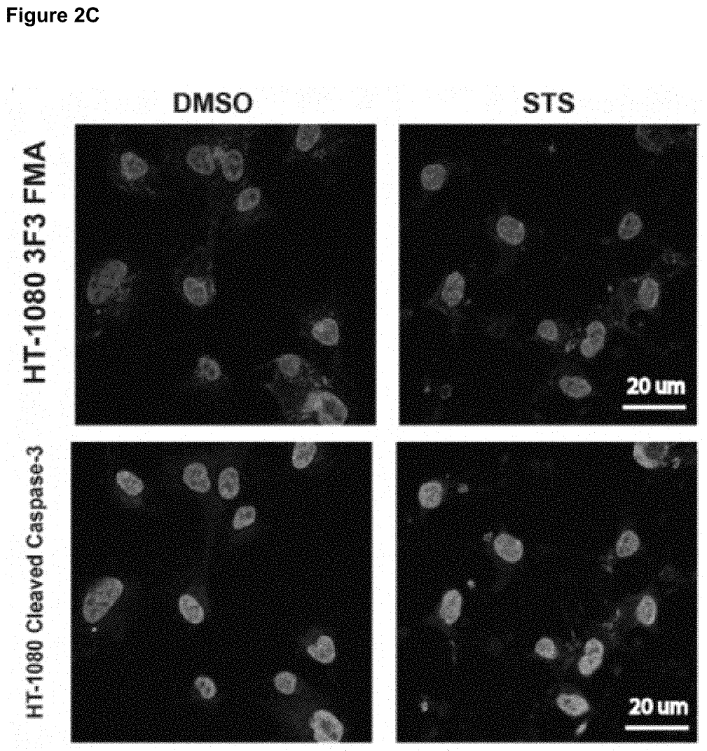

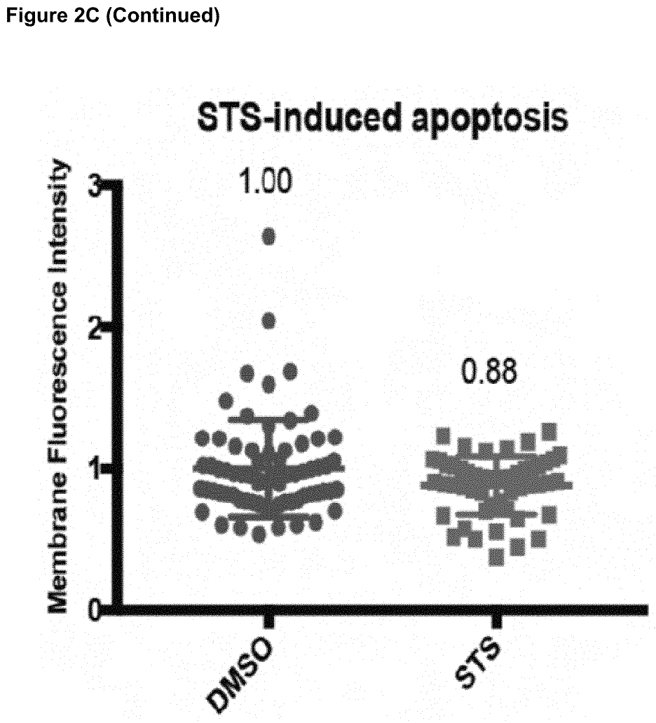

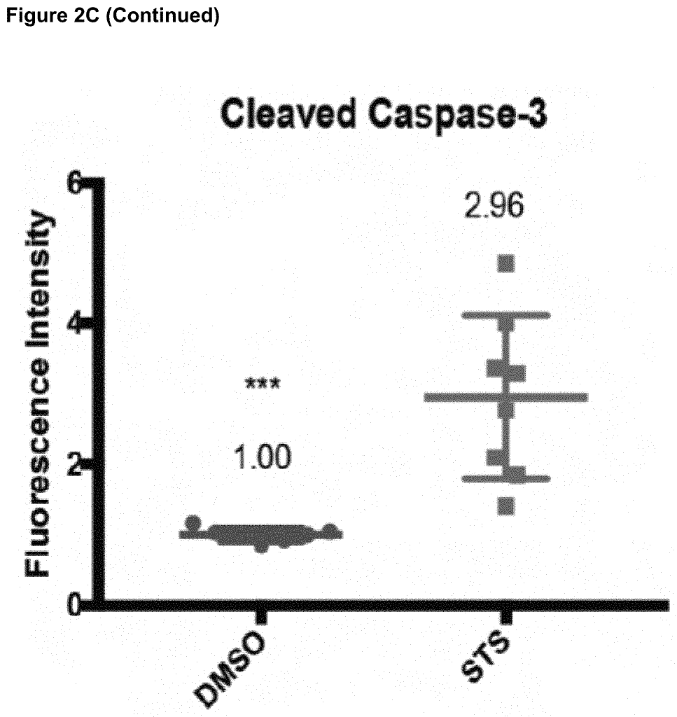

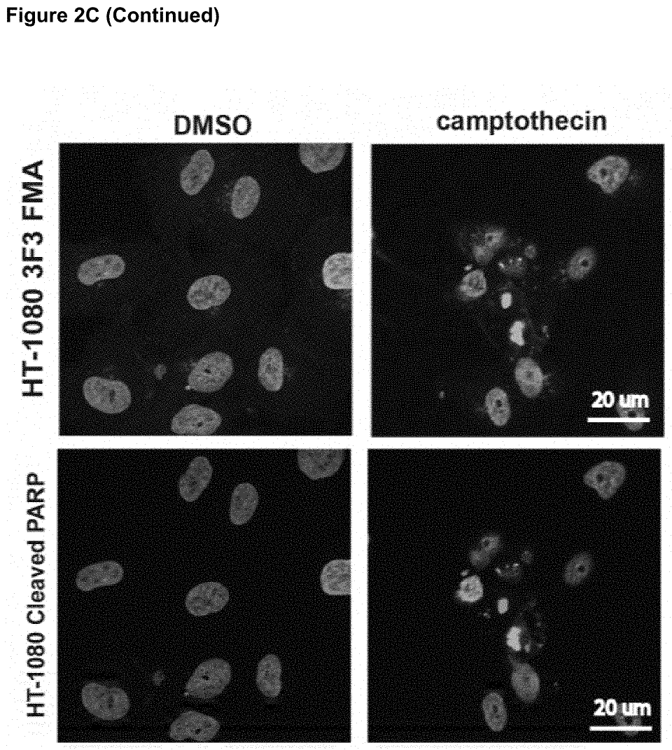

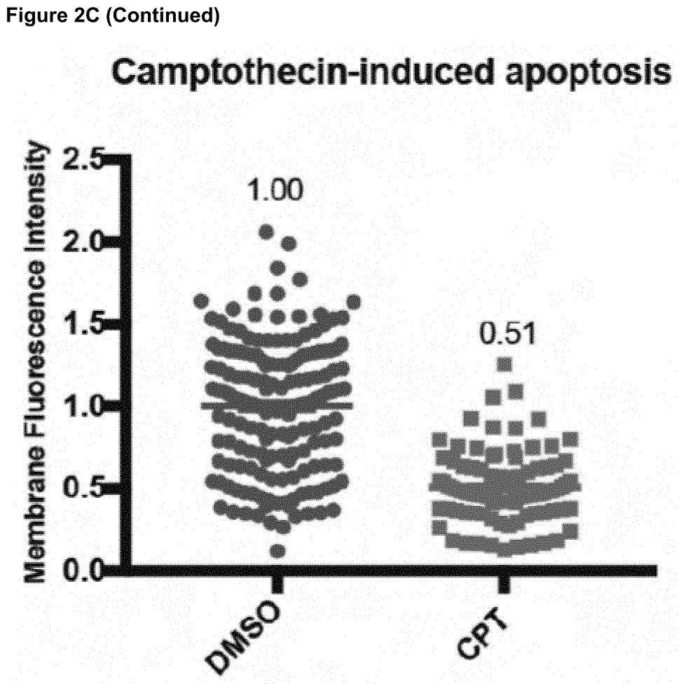

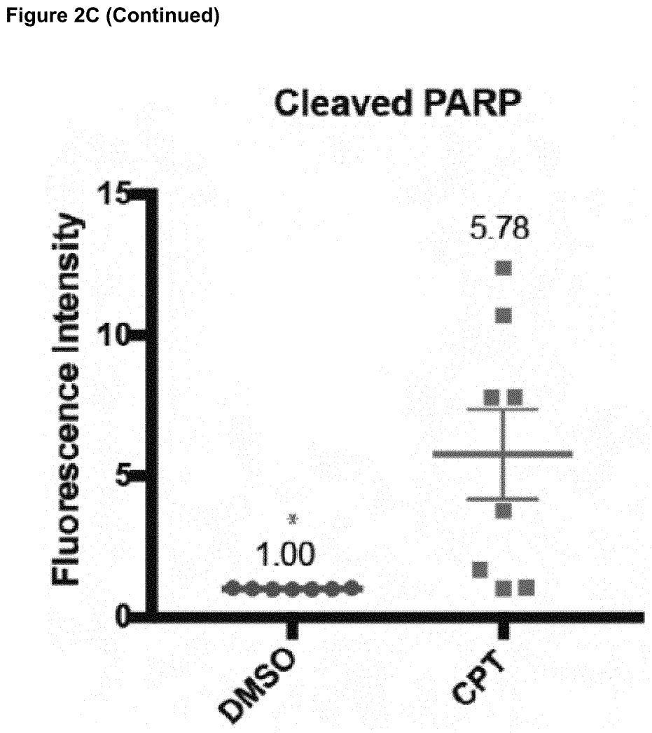

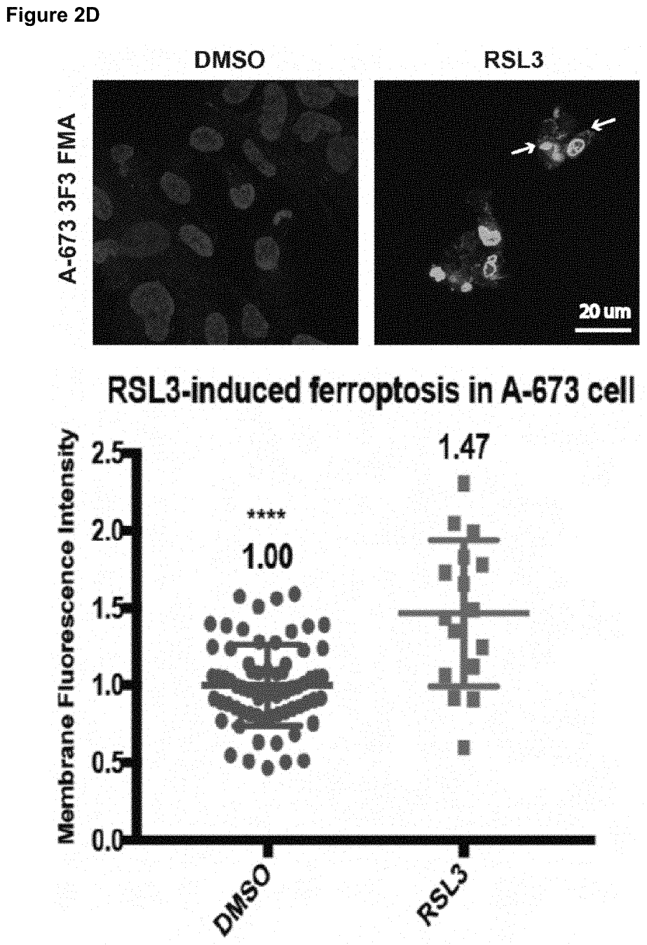

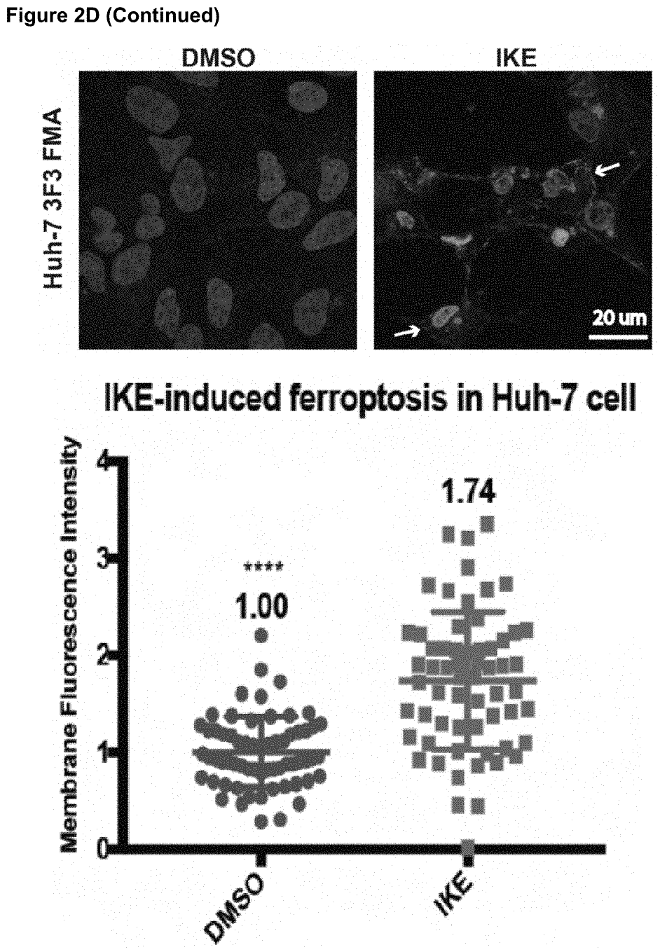

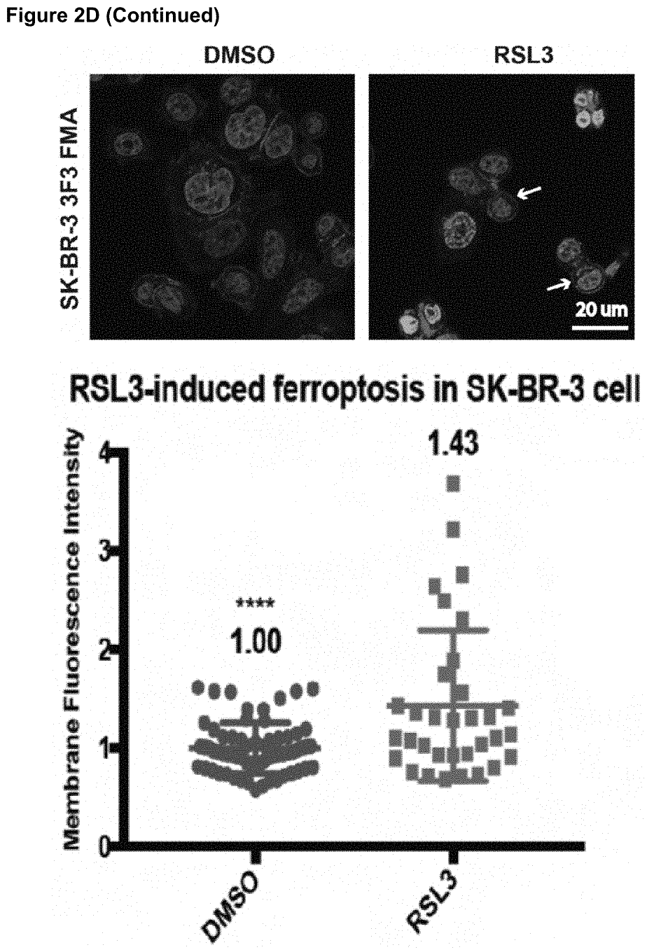

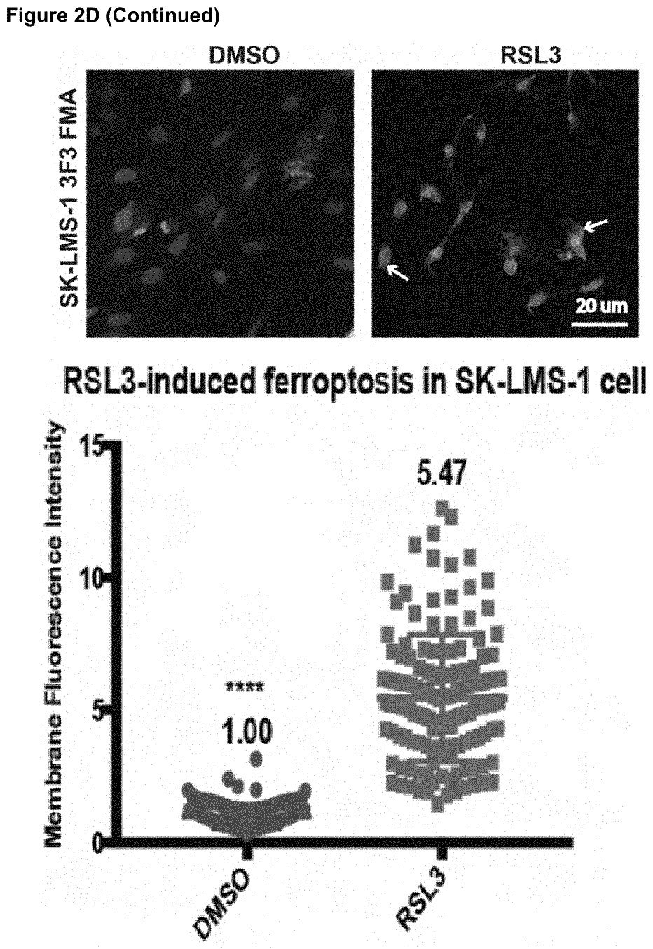

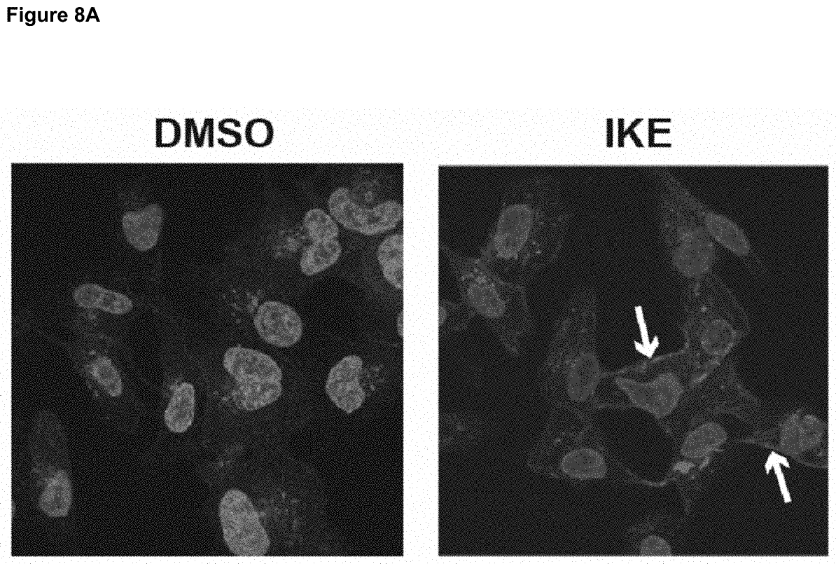

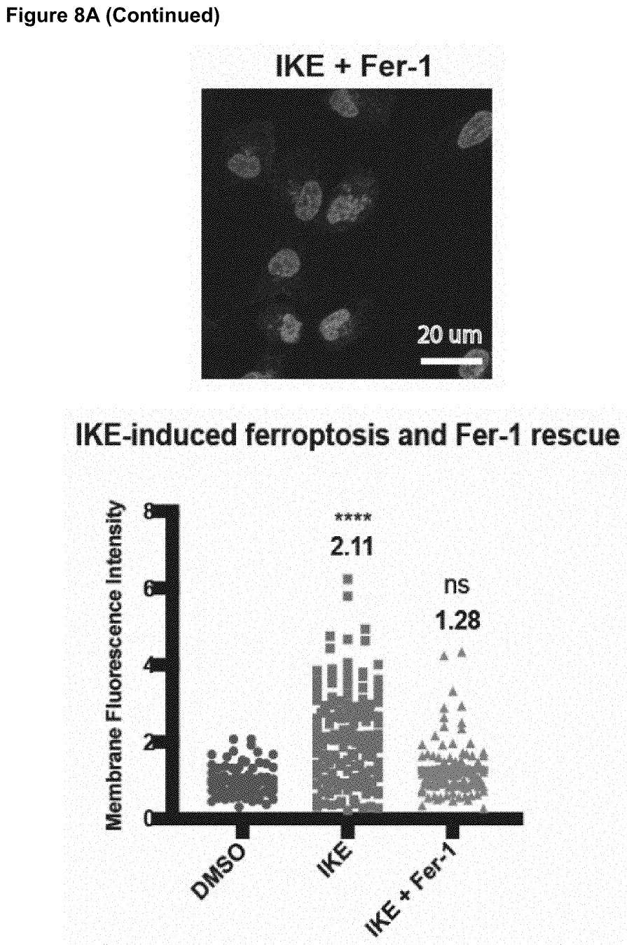

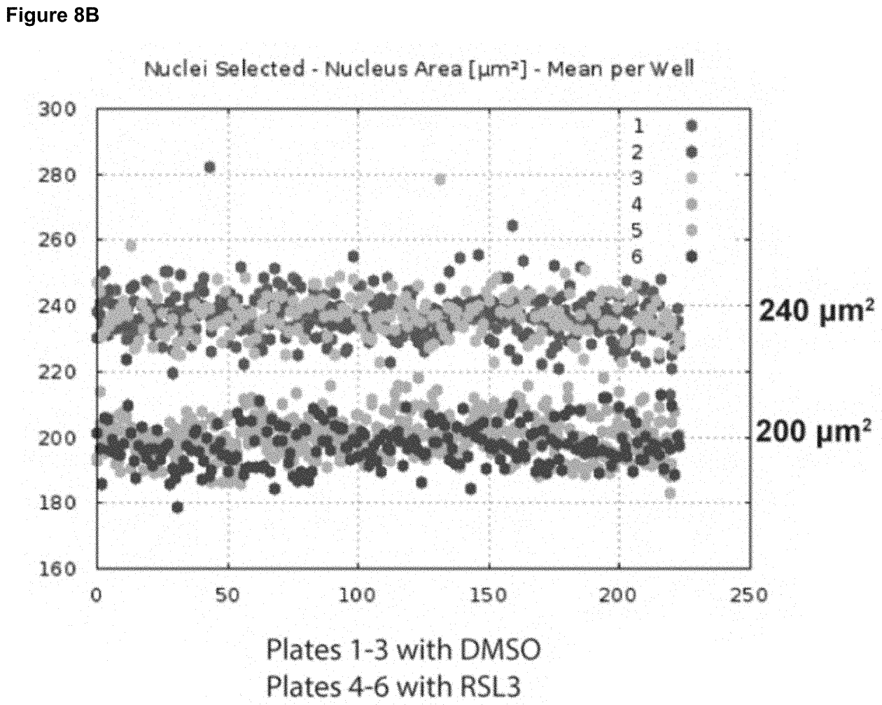

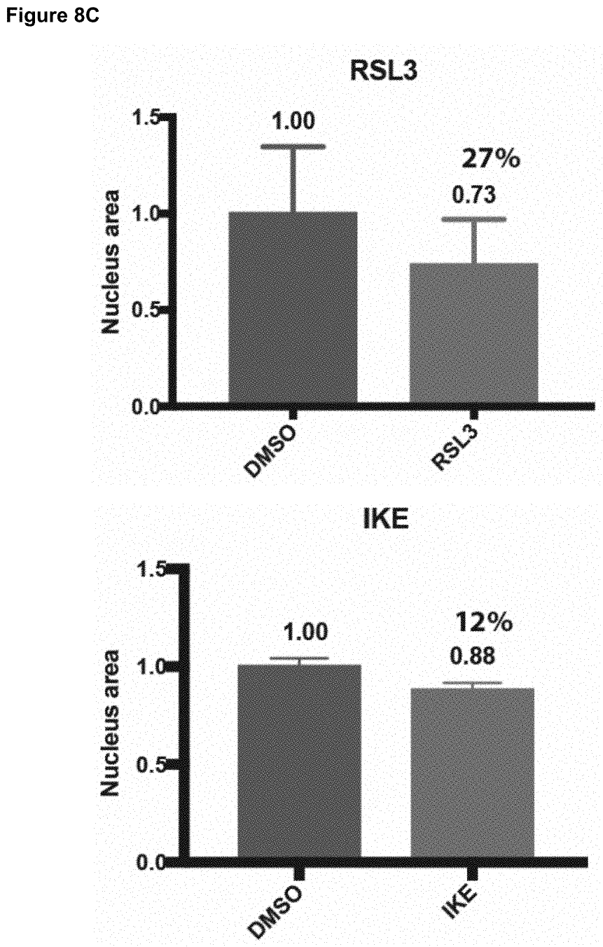

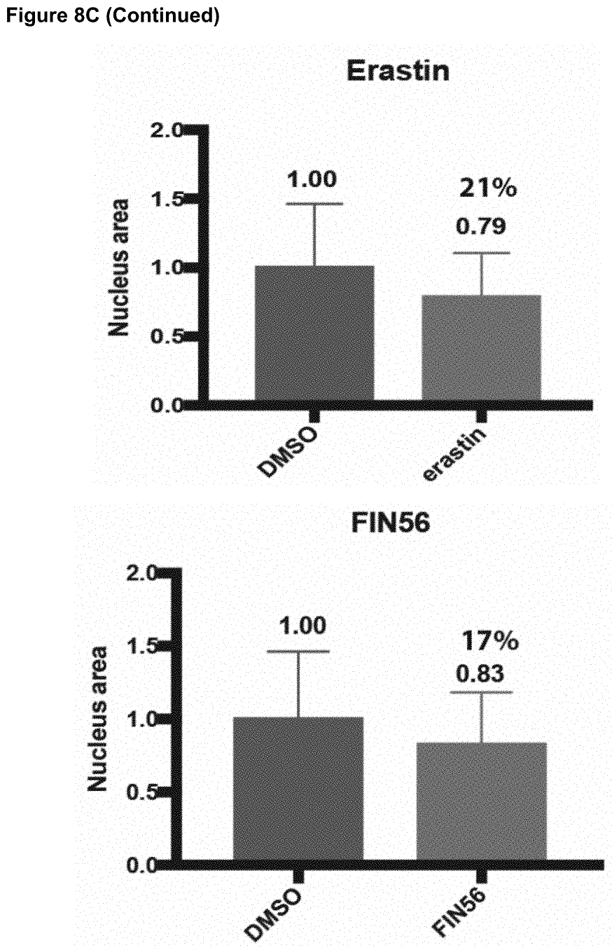

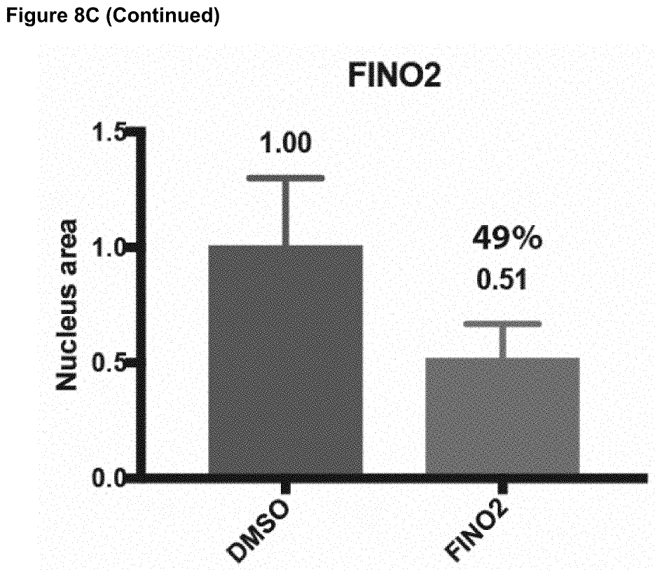

The three hits were verified on a larger scale by immunofluorescence using confocal microscopy. We used RSL3 in an initial validation experiment, because it's the most potent inducer, requiring low concentration and short incubation times. 30% of HT-1080 cells died after 4 h incubation of 1 μM RSL3. 3F3-FMA was the only Ab showing reliable differences in staining during RSL3-induced ferroptosis. During ferroptosis, 3F3-FMA stained cell boundaries and the inside puncta became brighter (FIG. 2A). To further validate 3F3-FMA as detecting ferroptotic cells, we added ferroptosis inhibitor fer-1 together with RSL3 (FIG. 2A) or IKE (FIG. 8A). The staining by 3F3-FMA in IKE+Fer-1 treated cells was similar to DMSO-treated cells, consistent with 3F3-FMA being able to detect cells undergoing ferroptosis.

Quantification of membrane fluorescence intensity was subsequently used to evaluate the difference in membrane-localized antigen. We used various ferroptosis inducers—IKE, erastin, FIN56, FINO2 and tBuOOH, and the same changes of 3F3-FMA pattern were observed (FIG. 2B). To test whether 3F3-FMA could differentiate ferroptosis from apoptosis, we used staurosporine (STS) to induce apoptosis, and used cleaved caspase-3 antibody as a marker of apoptosis. Accumulation of 3F3-FMA staining on the cell surface wasn't detected, indicating that the staining changes detected by 3F3-FMA were specific to ferroptosis over apoptosis (FIG. 2C). To further test the ferroptosis specificity of 3F3 FMA, camptothecin was used to induce apoptosis in HT-1080 cells, with cleaved PARP antibody as an apoptosis marker. Similar to STS-treated cells, there was no significant increase in 3F3-FMA accumulation on the cell surface, further indicating that this accumulation pattern is specific to ferroptosis.

Next, this validation was expanded to other cancer cell lines. We selected A-673 (human muscle sarcoma cells), SK-BR-3 (human breast cancer cells), Huh-7 (hepatocyte-derived carcinoma cells) and SK-LMS-1 (human leiomyosarcoma cells) which are all sensitive to ferroptosis. Again, we found that 3F3-FMA staining concentrated at the cell boundaries and became brighter upon ferroptosis induction (FIG. 2D). Taken together, the data indicate that the 3F3-FMA antibody can be used as a ferroptosis marker using immunofluorescence microscopy with cells in culture.

Similar to apoptosis, nuclear shrinkage under ferroptosis was also reproducibly found, with a 17% decrease in nuclear size (FIG. 8B). We validated this finding by immunofluorescence (FIG. 8C). The extent of shrinkage depended on the kind of inducers and incubation time. The nuclei shrank by 27% in 4 h incubation of 1 μM RSL3, 12% in 8 h incubation of 10 μM IKE, 21% in 8 h incubation of 15 μM erastin, 17% for an 8 h incubation of 10 μM FIN56 and 49% during an 8 h incubation of 15 μM FINO2. This provides another morphological feature of ferroptosis.

### Example 4

### The Antigen of 3F3-FMA is Transferrin Receptor 1

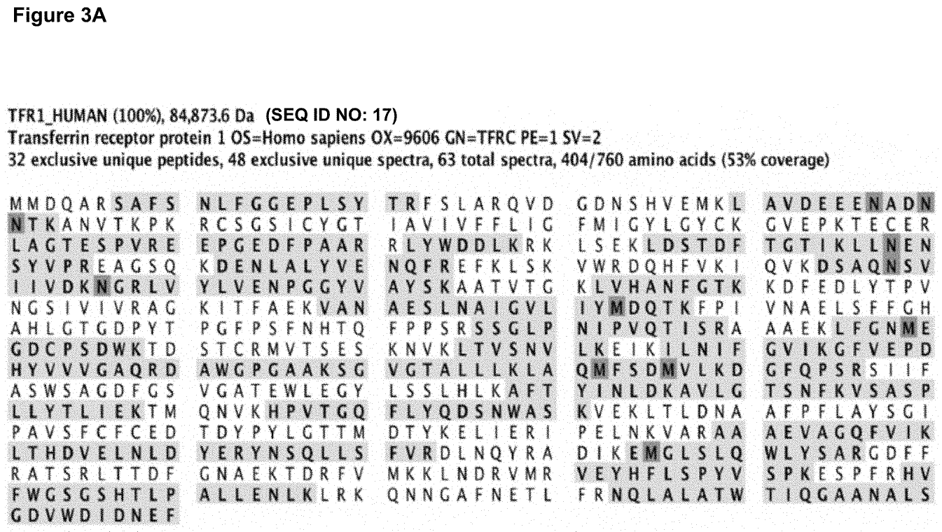

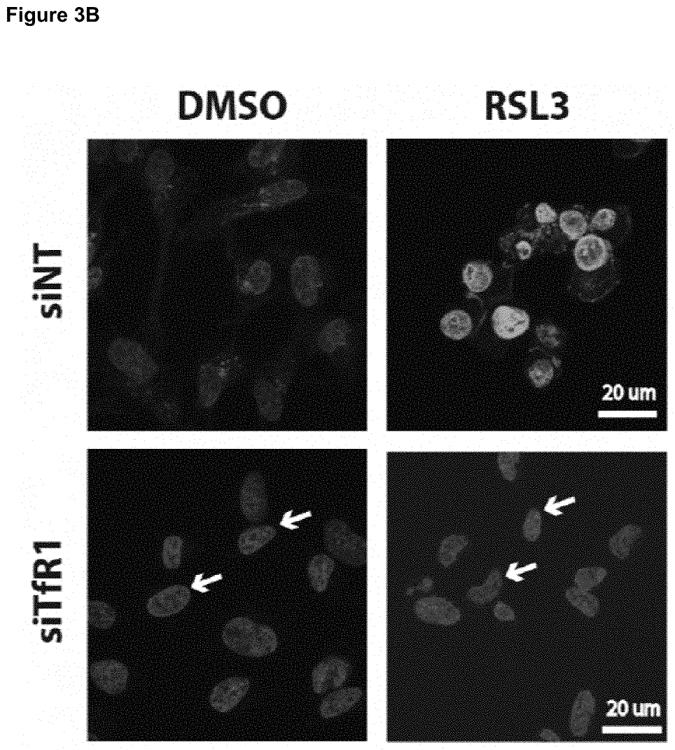

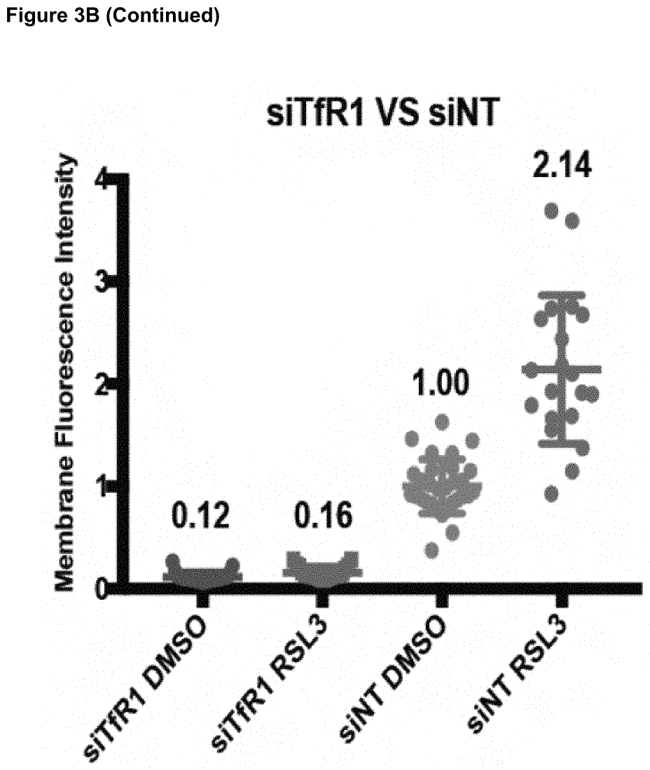

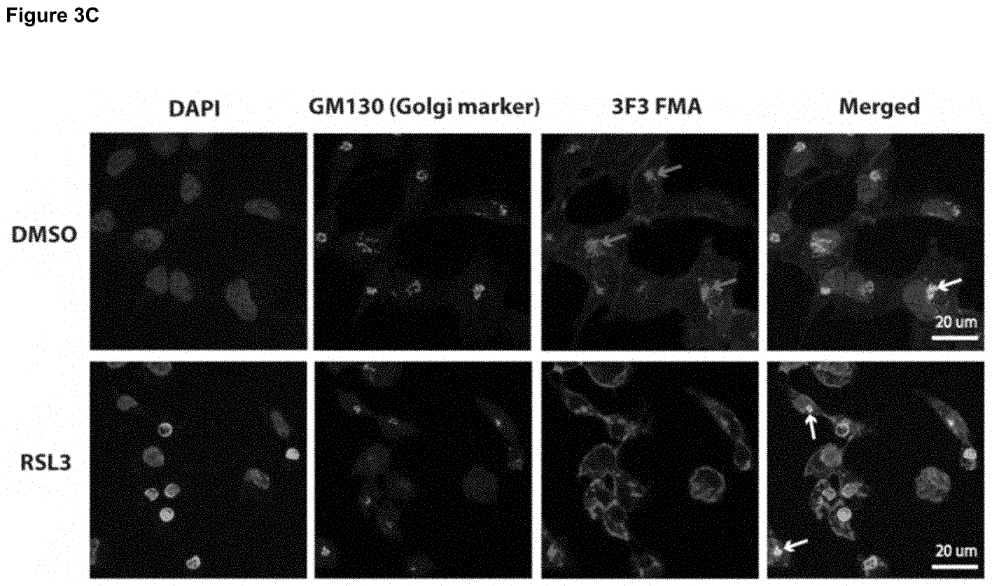

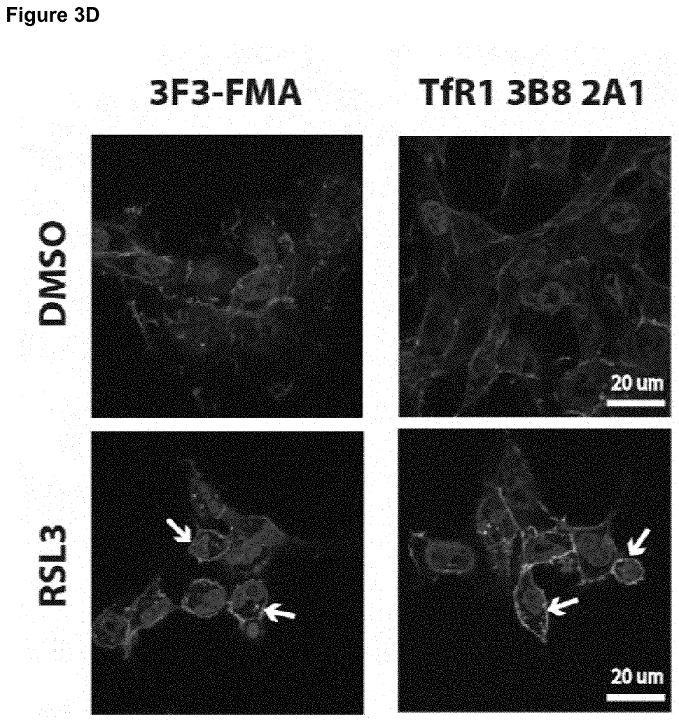

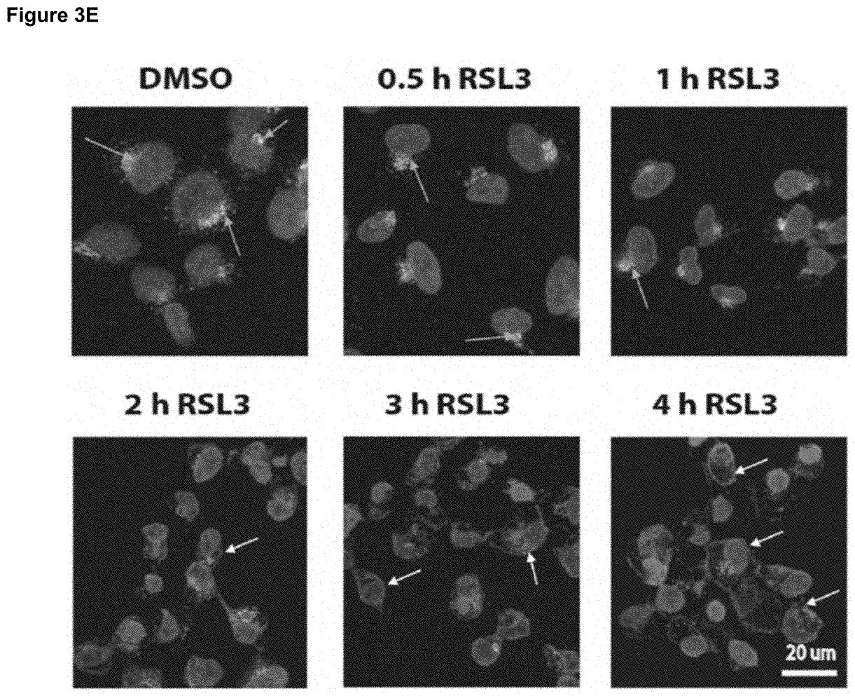

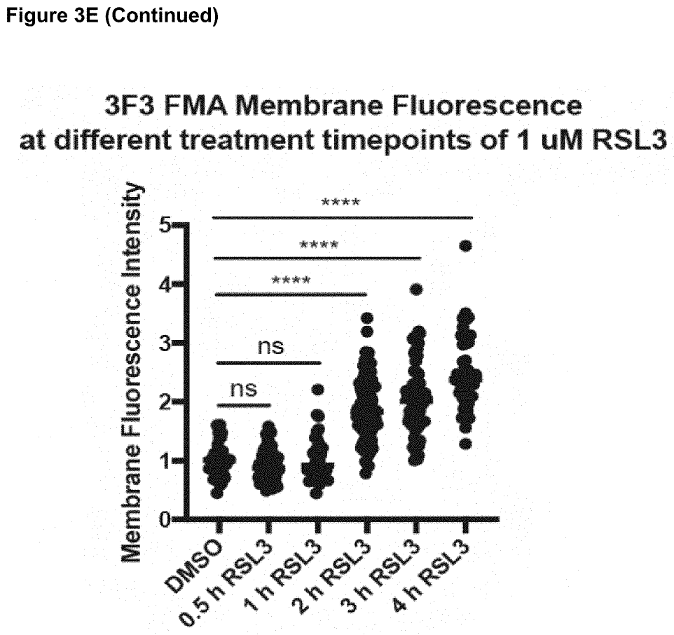

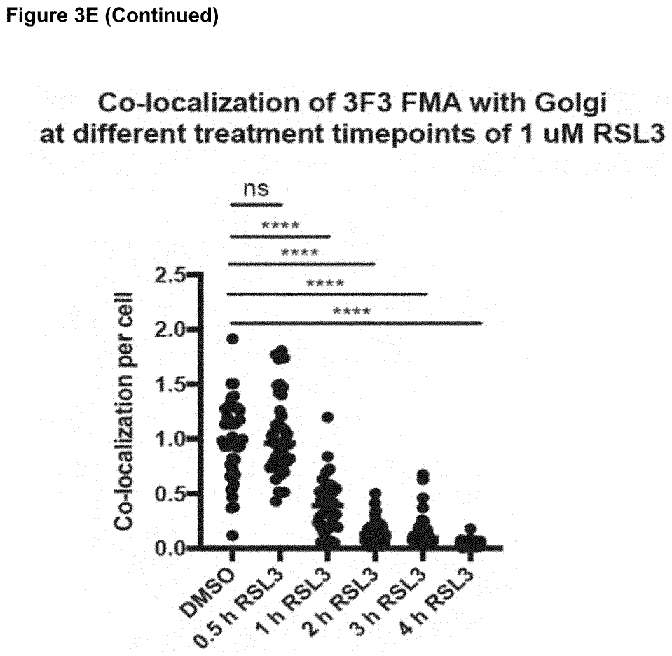

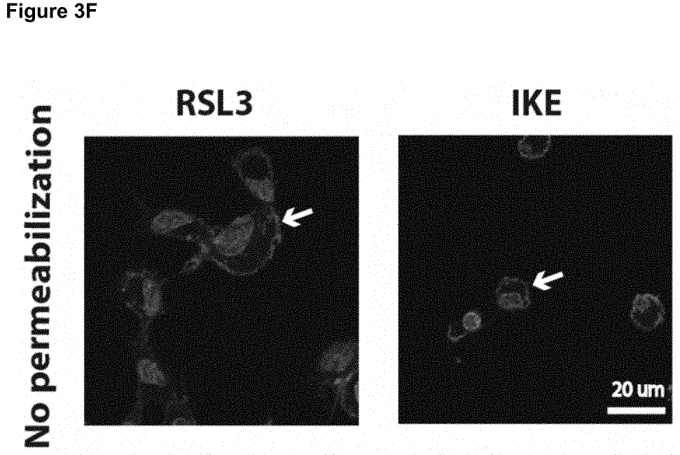

The antigen of 3F3-FMA was identified using immunoprecipitation and mass spectrometry. First, we incubated 3F3-FMA with a cell lysate overnight. Then magnetic beads were added and washed, and bead-immobilized proteins were analyzed by mass spectrometry. Transferrin receptor protein 1 had the highest confidence identification as the target antigen at 63% with 32 exclusive unique peptides, 48 exclusive unique spectra and 53% amino acid coverage (FIG. 3A). We further validated this antigen by siRNA knockdown: siRNAs targeting TfR1 versus NT (non-targeting) were transfected into HT-1080 cells and incubated for 48 h. After an additional 24 h, cells were fixed and stained with 3F3-FMA and nuclei. In siTfR1-transfected cells, there wasn't any detectable staining by 3F3-FMA, supporting the argument that the target antigen is transferrin receptor protein 1 (FIG. 3B).

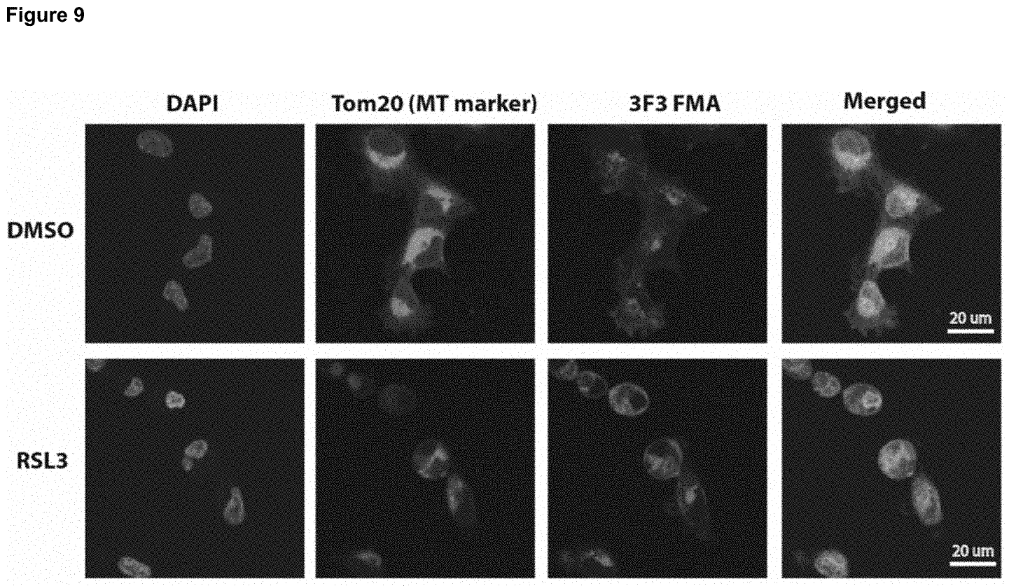

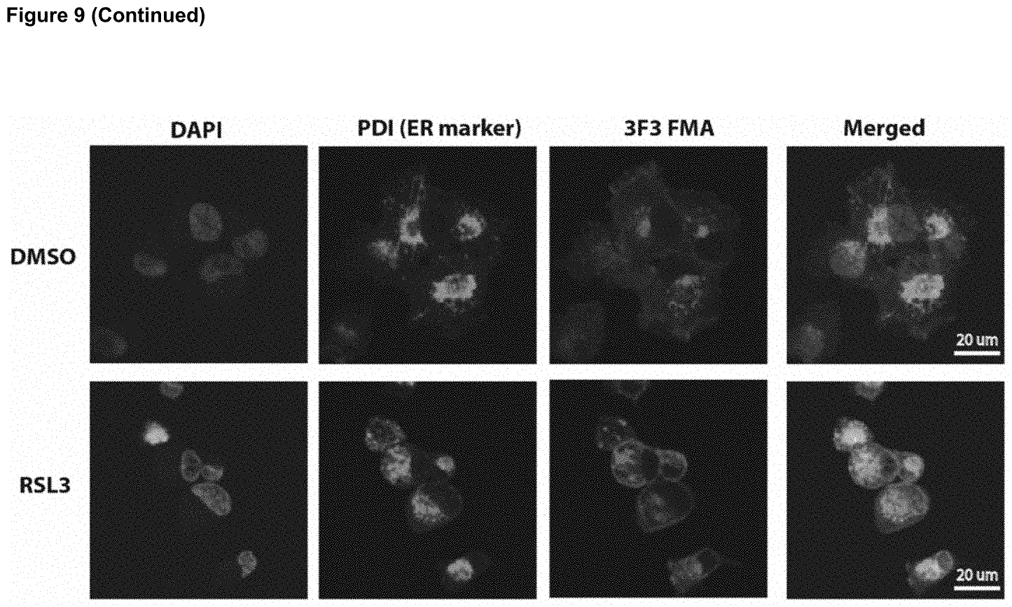

Next, to better localize 3F3-FMA staining within cells, 3F3-FMA was co-localized with Tom20 (mitochondria marker), PDI (ER marker) and GM130 (Golgi marker) using two secondary antibodies with different excitation and emission wavelengths. It's found that 3F3-FMA staining was not visible in mitochondria or the ER (FIG. 9). 3F3-FMA staining was instead located in the Golgi (FIG. 3C). In untreated conditions, most staining remained in the Golgi (bright dots shown by green arrows), but when ferroptosis took place, these puncta moved out of the Golgi (FIG. 3C). We further determined that these puncta moved to the plasma membrane, by co-staining 3F3-FMA or TfR1 3B8 2A1 antibodies with a wheat germ agglutinin (WGA) Alexa Fluor™ 633 conjugate, a commonly used reagent to label glycoproteins for imaging of the plasma membrane (FIG. 3D). We monitored translocation by examining multiple time points upon the induction of ferroptosis by RSL3, and co-staining TfR1 with a Golgi marker. Increased accumulation of TfR1 protein in the plasma membrane and decreased localization in the Golgi was observed during the course of RSL3 treatment (FIG. 3E). This translocation was further confirmed by fixing cells without permeabilization: we found that the 3F3-FMA antibody could stain the plasma membrane in cells fixed without permeabilization, confirming membrane translocation of the TfR1 antigen (FIG. 3F). The translocation of the 3F3-FMA antigen was consistent with the antigen being in the extracellular or transmembrane domains of TfR1 (Aisen, 2004).

TfR1 is the main regulator of iron uptake in cells. After binding to iron-loaded transferrin, TfR1 is enclosed within clathrin-coated endocytic vesicles and internalized by cells. Iron is then released due to endosomal acidification. Apotransferrin and its receptor are sorted in the Golgi and to some extent transported back to the cell surface. It was therefore consistent with the known trafficking of TfR1 that we observed the 3F3-FMA antigen at the plasma membrane and in the Golgi.

### Example 5

### Application of Transferrin Receptor 1 Antibodies in Immunofluorescence and Comparison with Other Potential Ferroptosis-Staining Reagents

In addition to 3F3-FMA, three other anti-TfR1 antibodies were acquired. Our goal was to test if these antibodies had similar staining pattern changes with 3F3-FMA, thereby further validating the TfR1 antigen. An additional goal was to compare these anti-TfR1 antibodies to see if one has advantages over others. To find the most specific and reliable ferroptosis marker, these TfR1 antibodies were compared with anti-MDA, anti-4-HNE and anti-ACSL4 antibodies, which have been explored as molecular markers of ferroptosis.

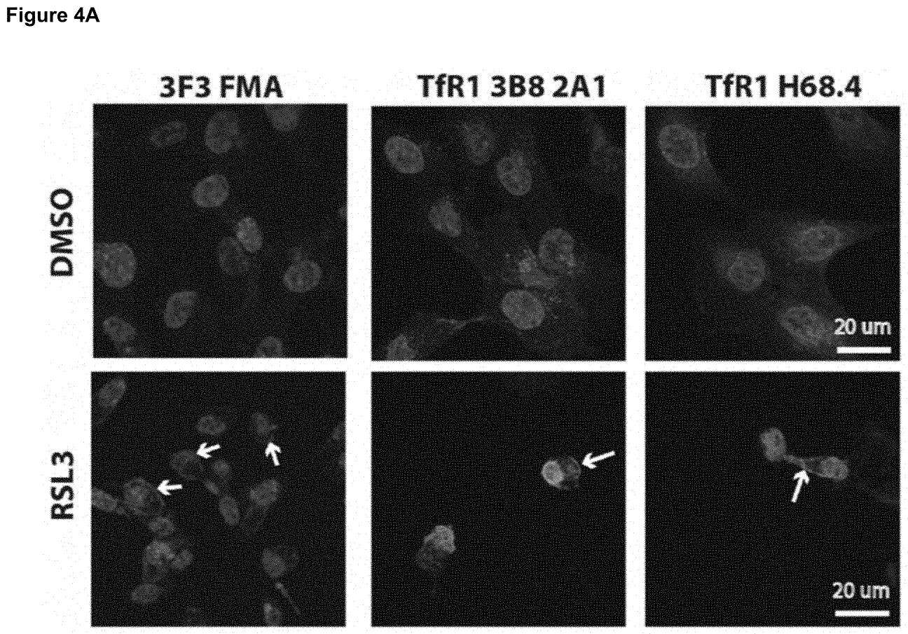

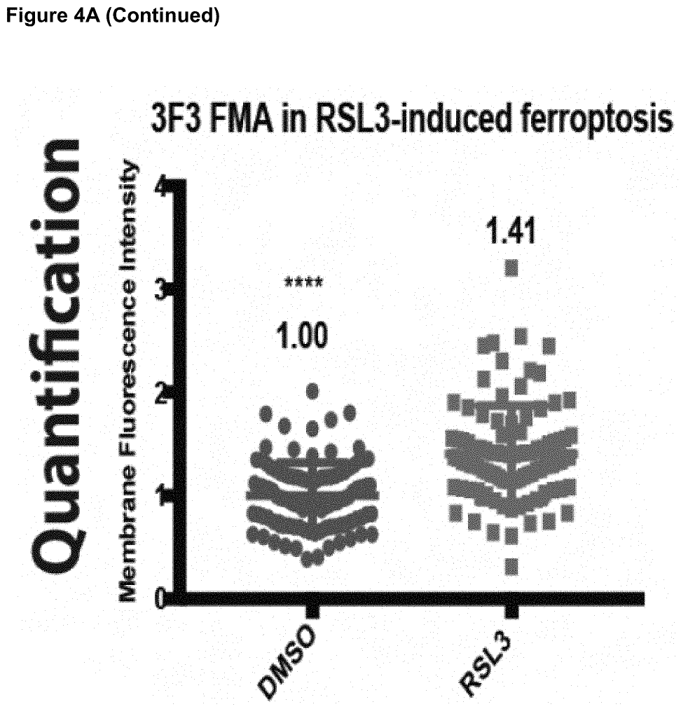

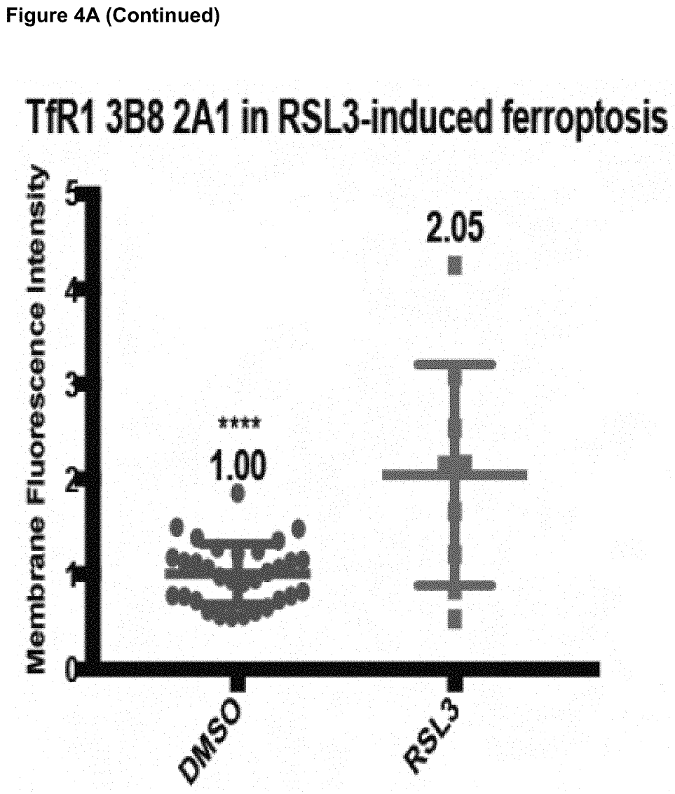

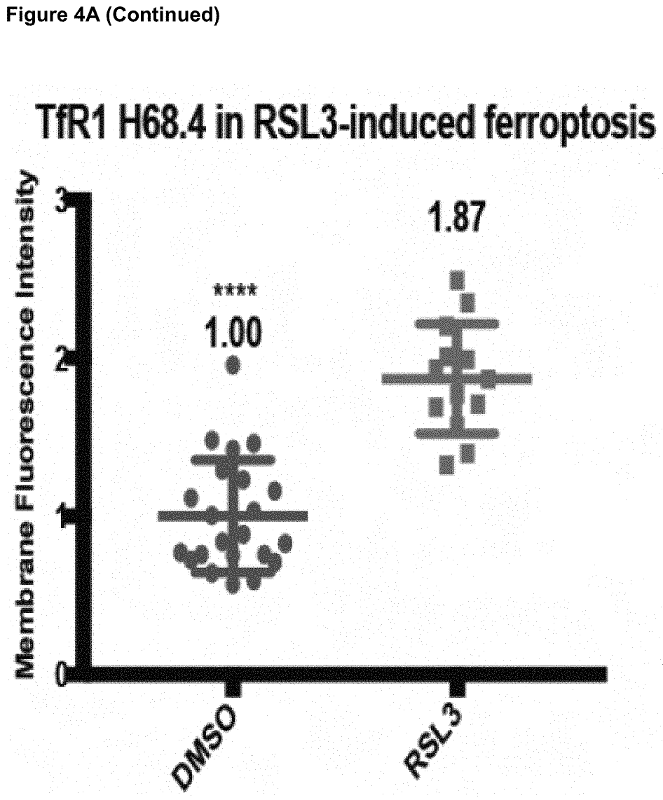

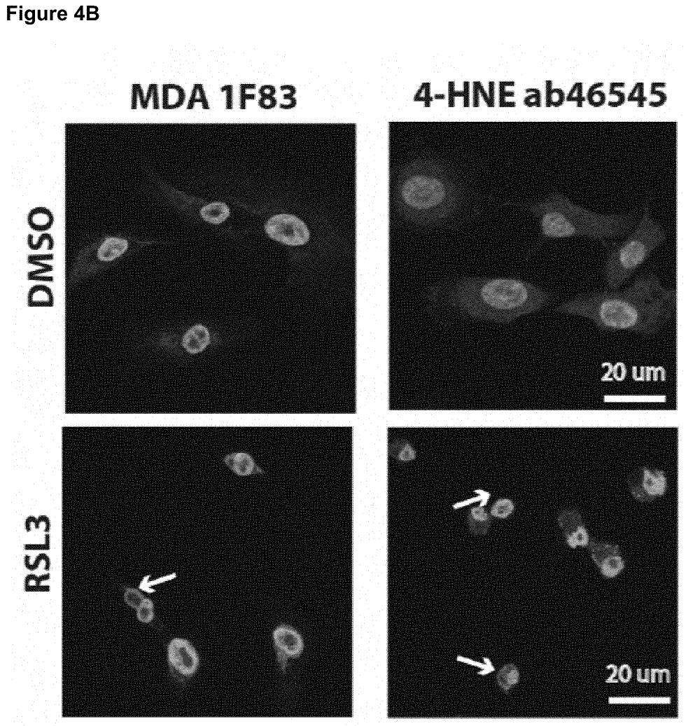

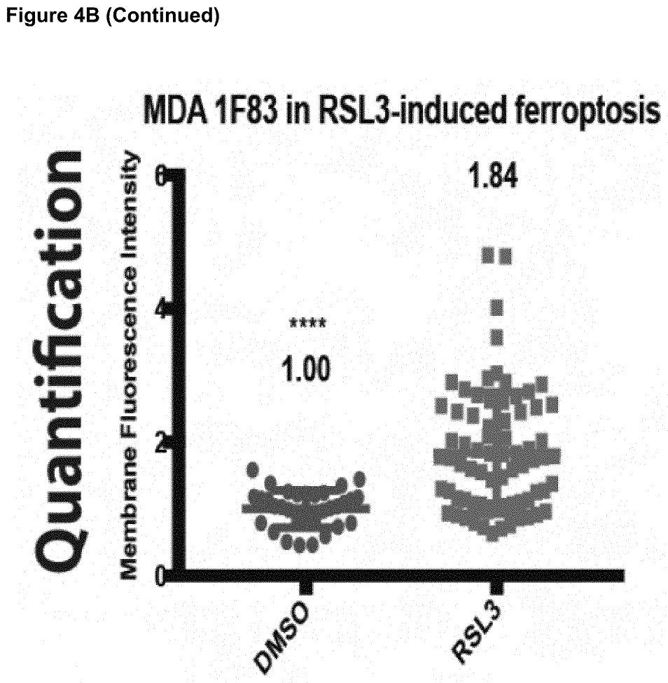

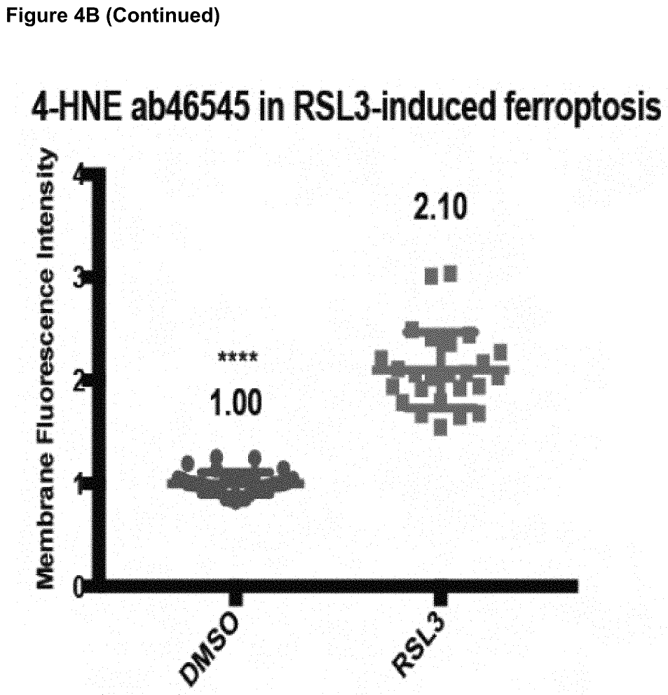

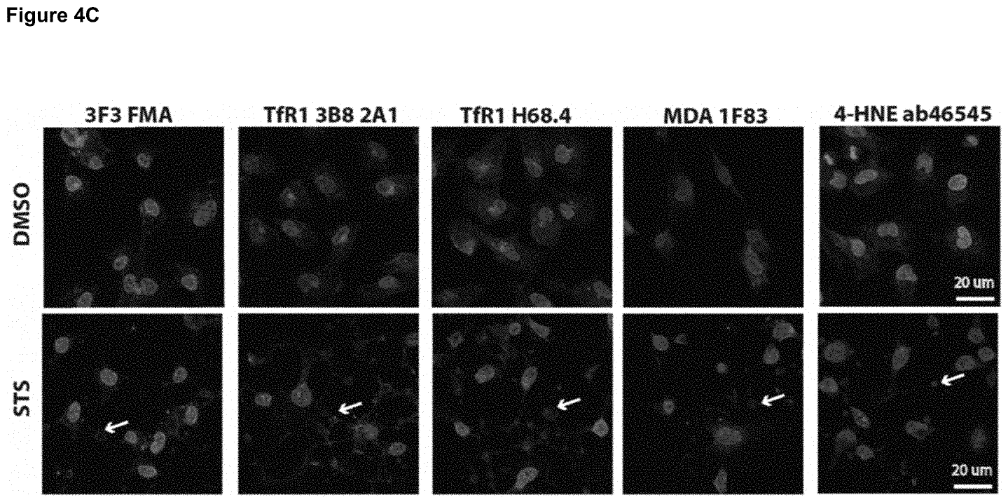

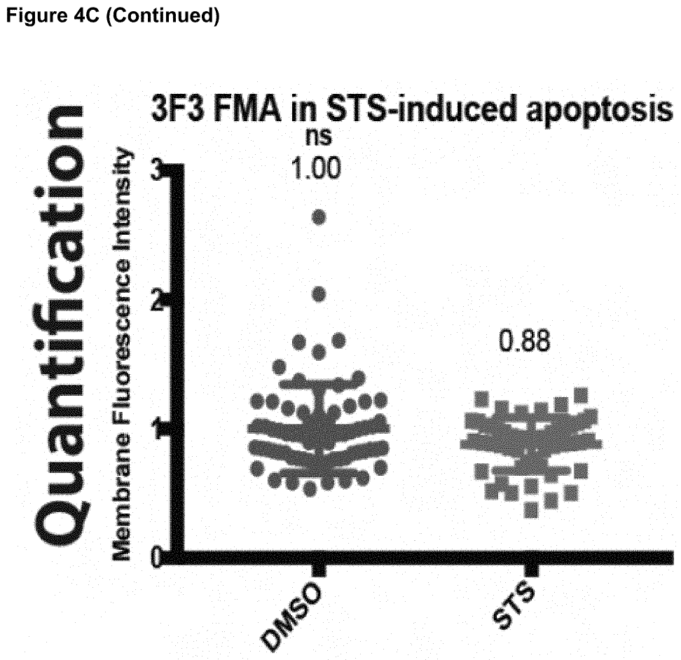

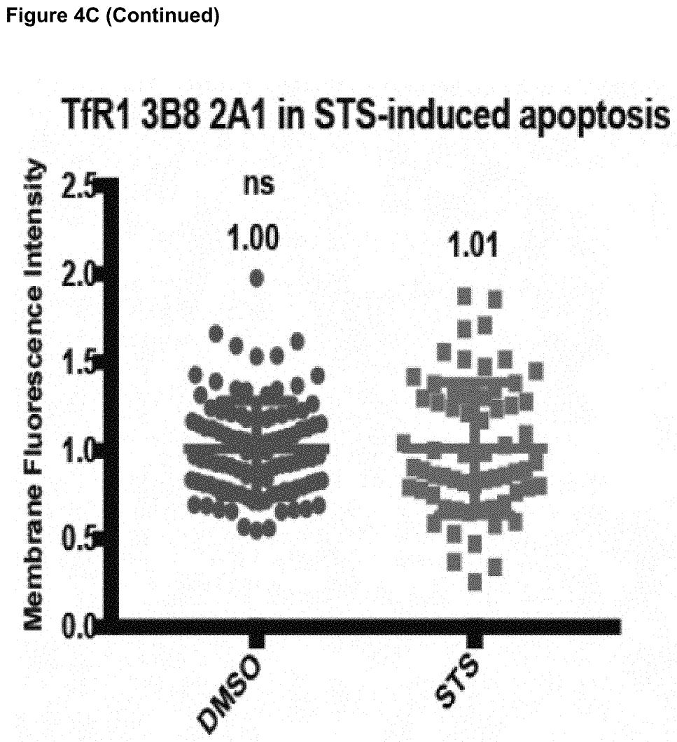

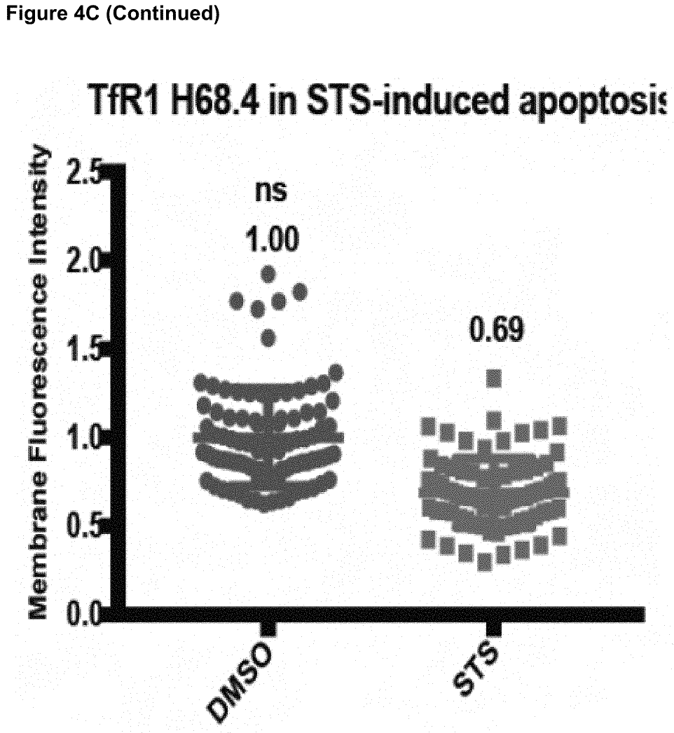

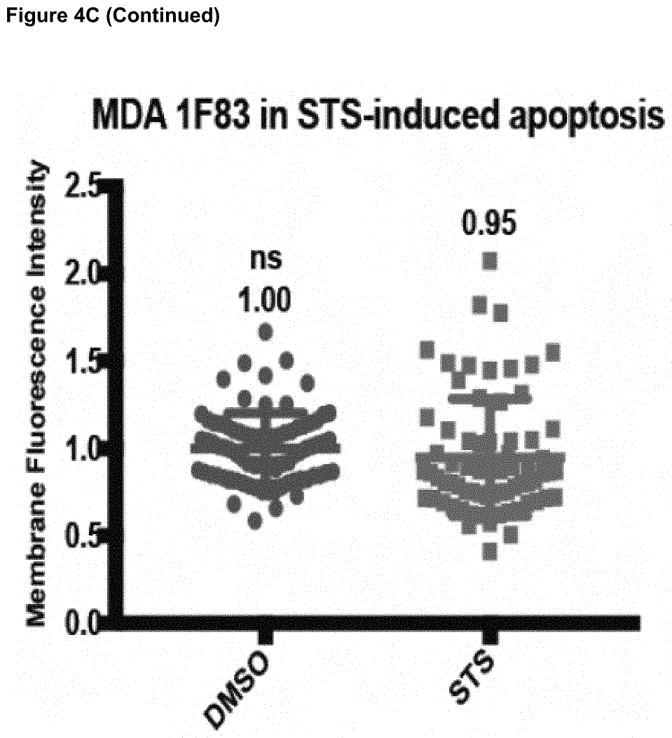

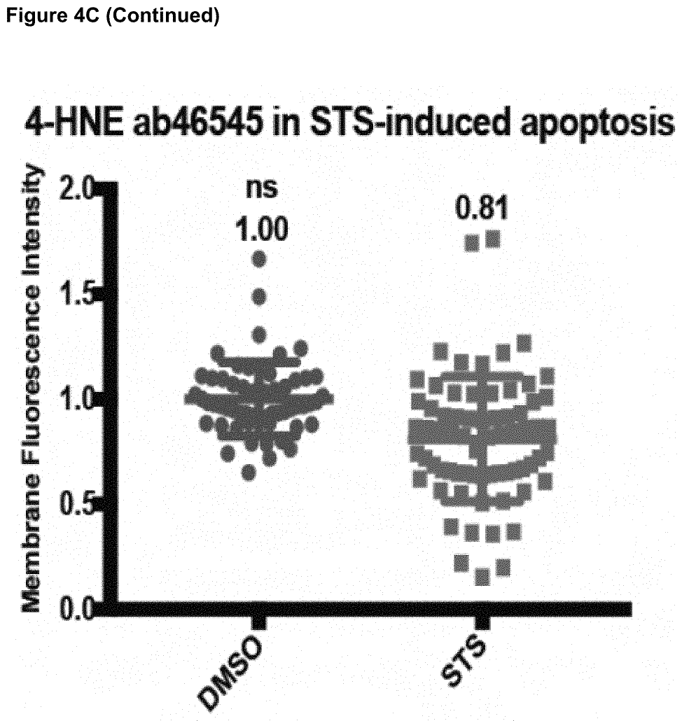

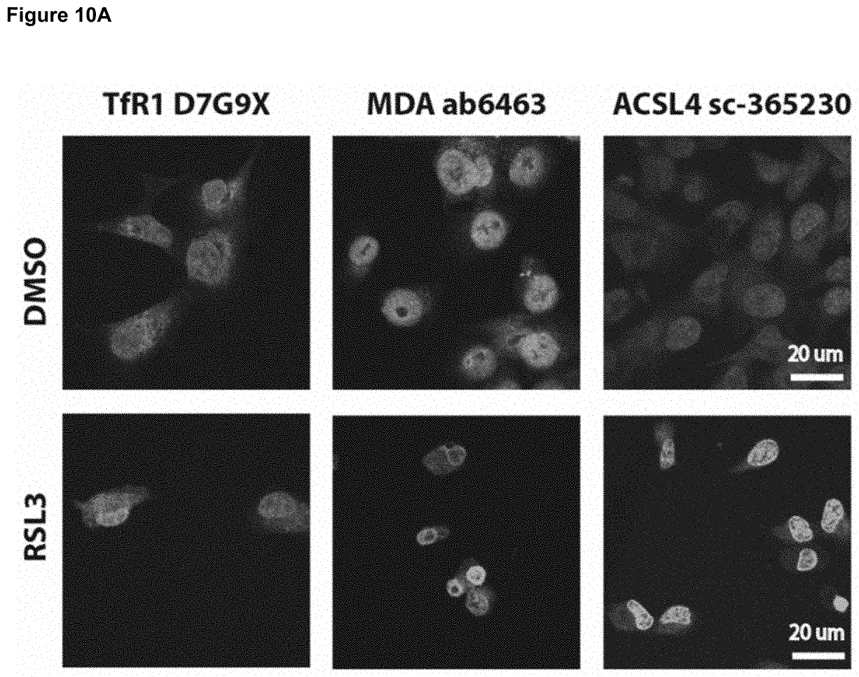

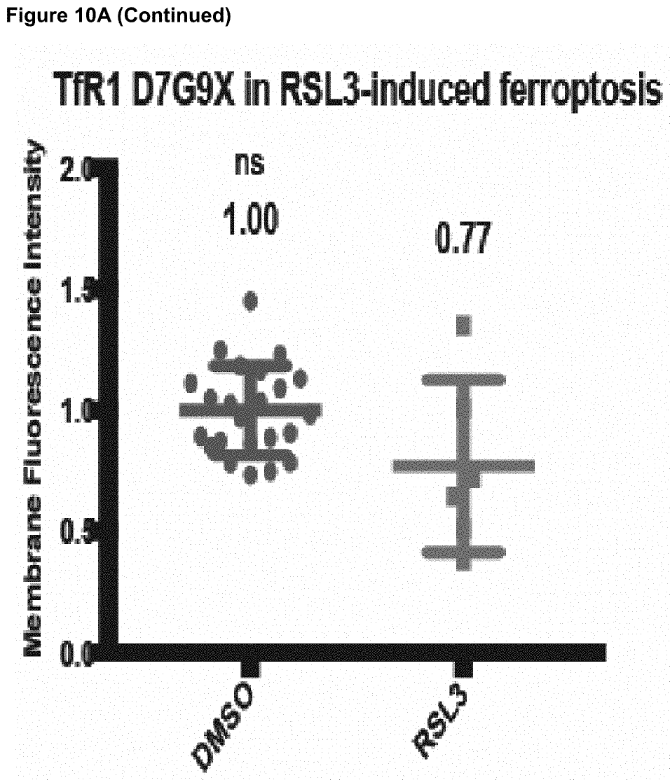

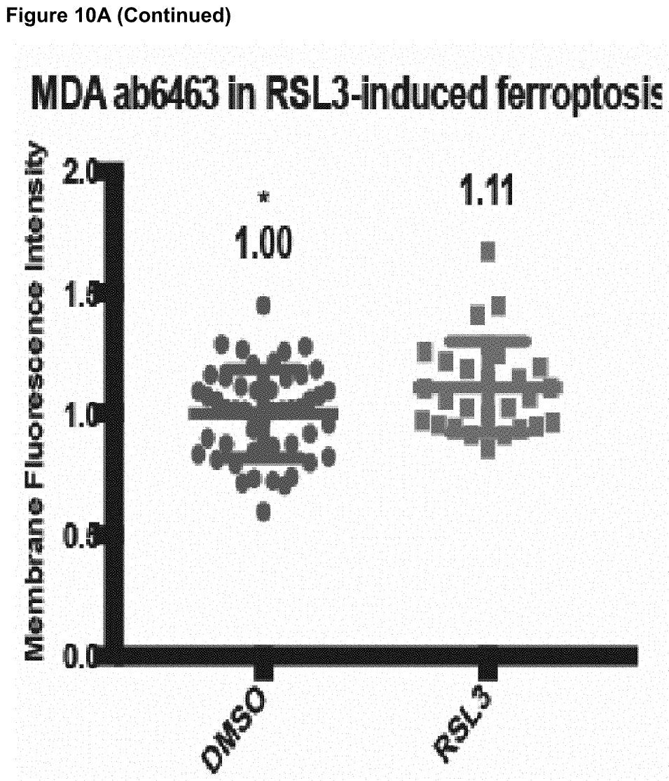

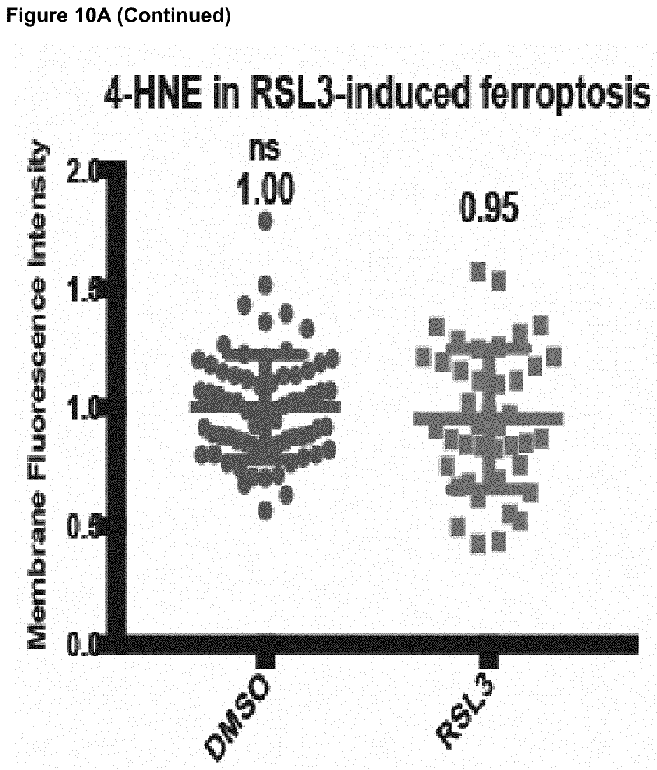

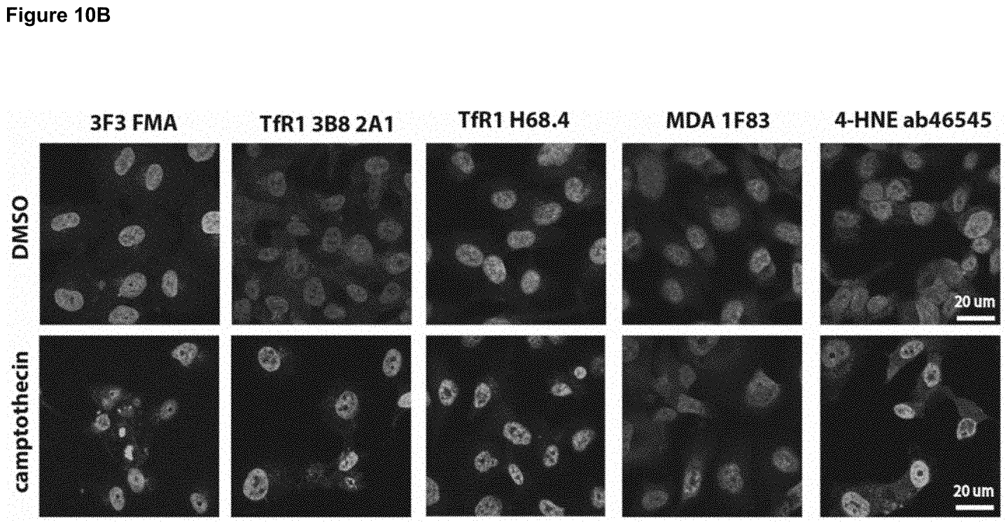

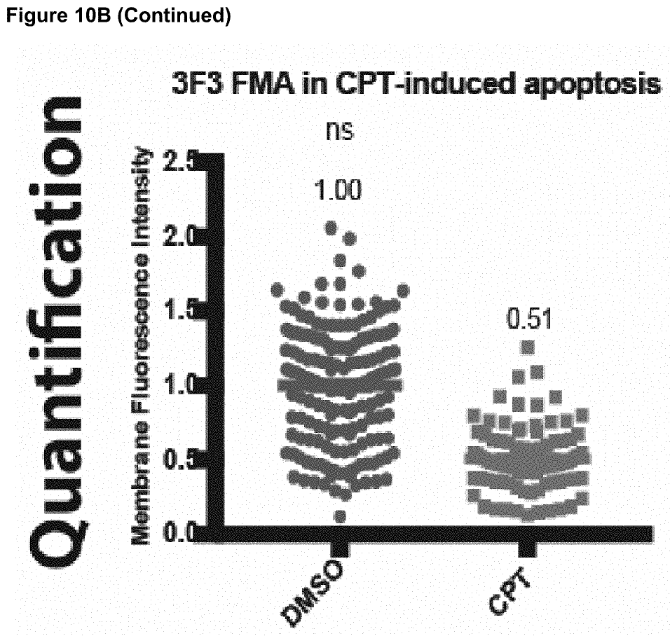

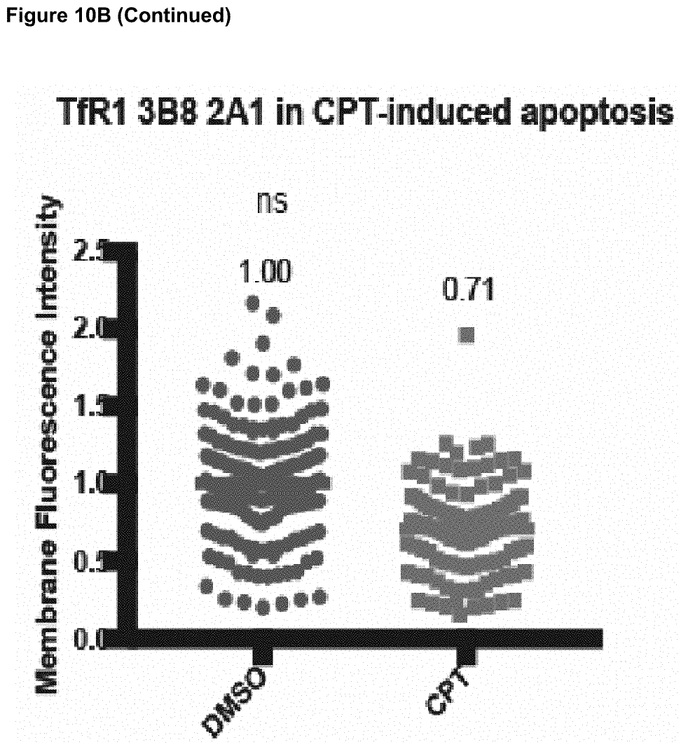

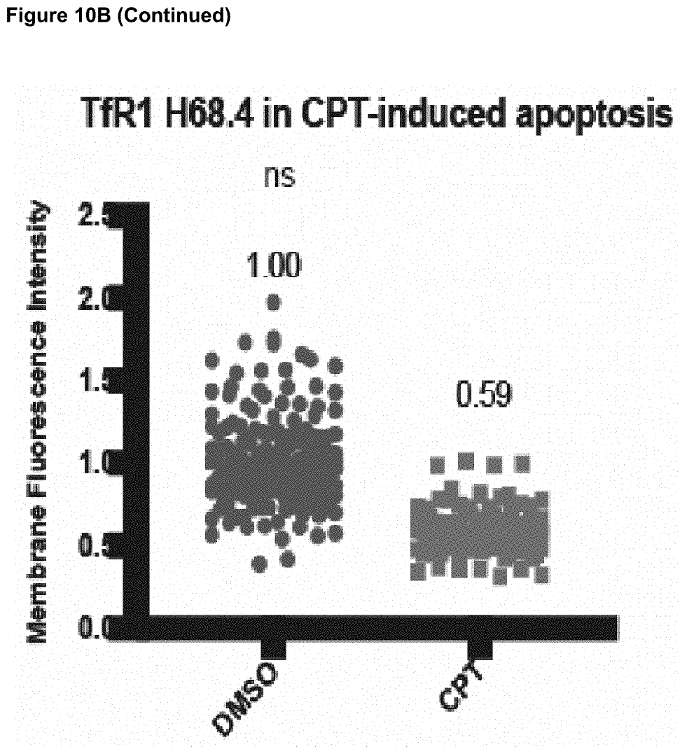

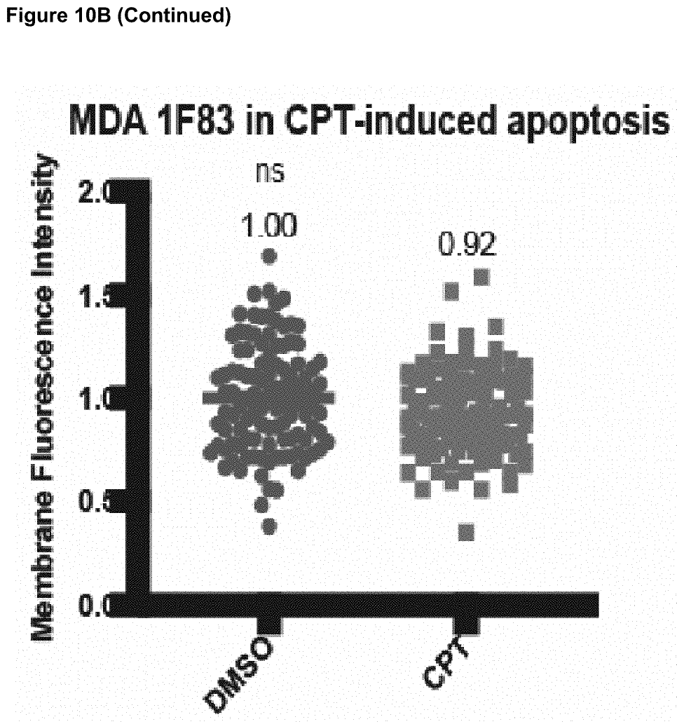

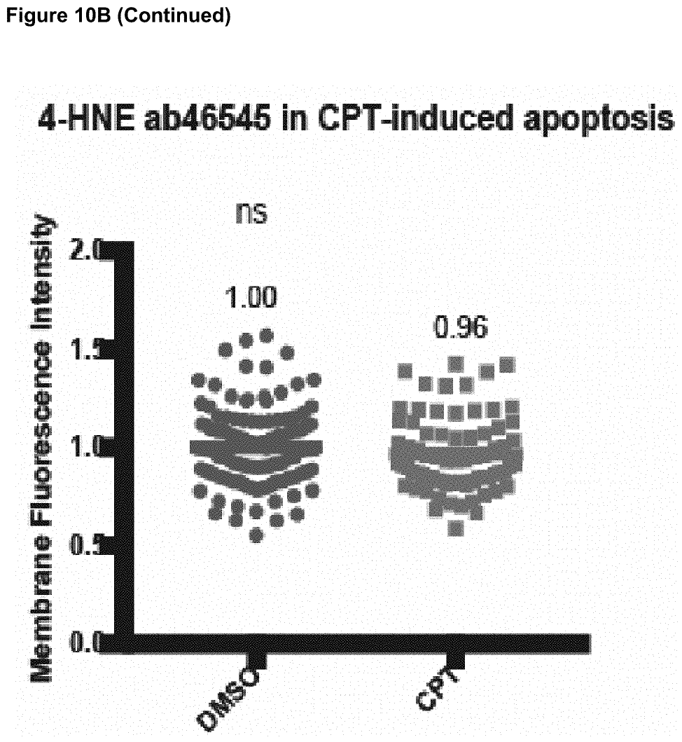

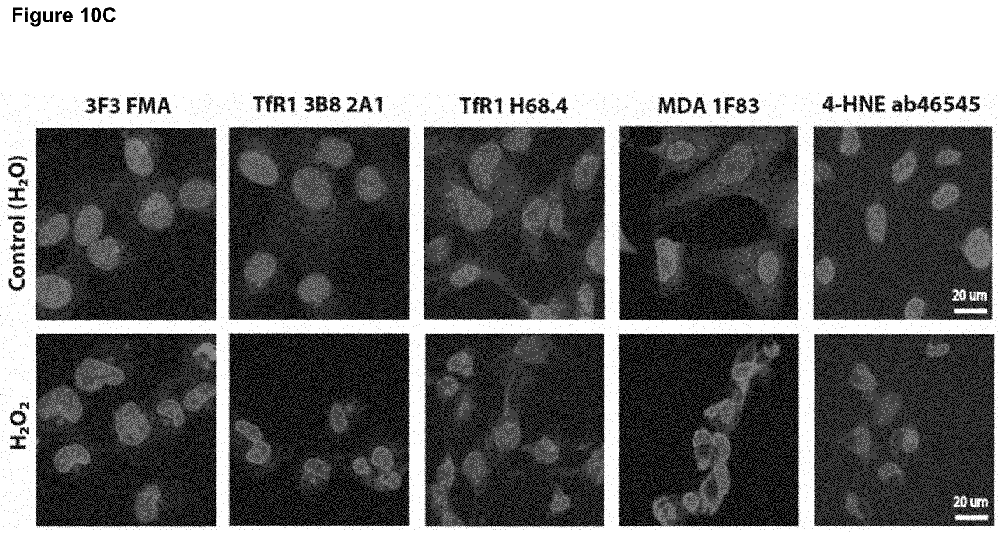

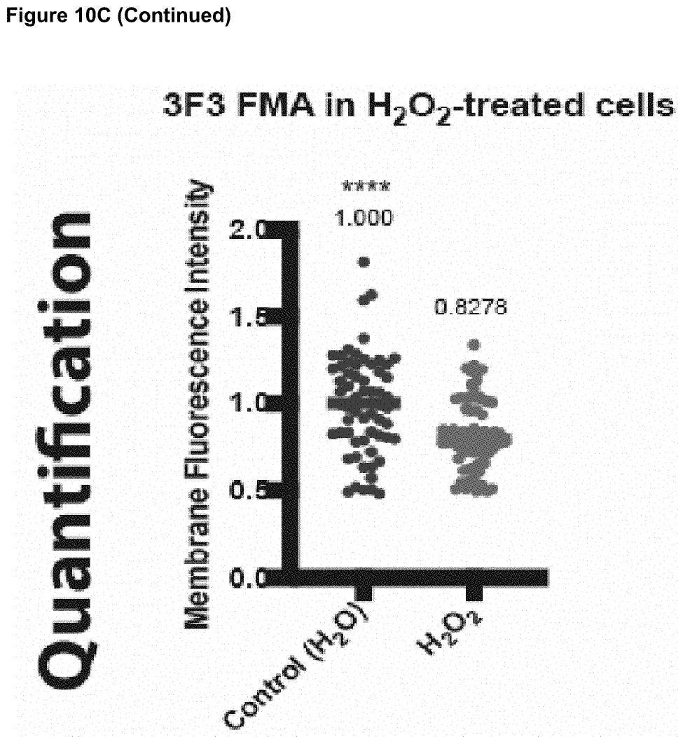

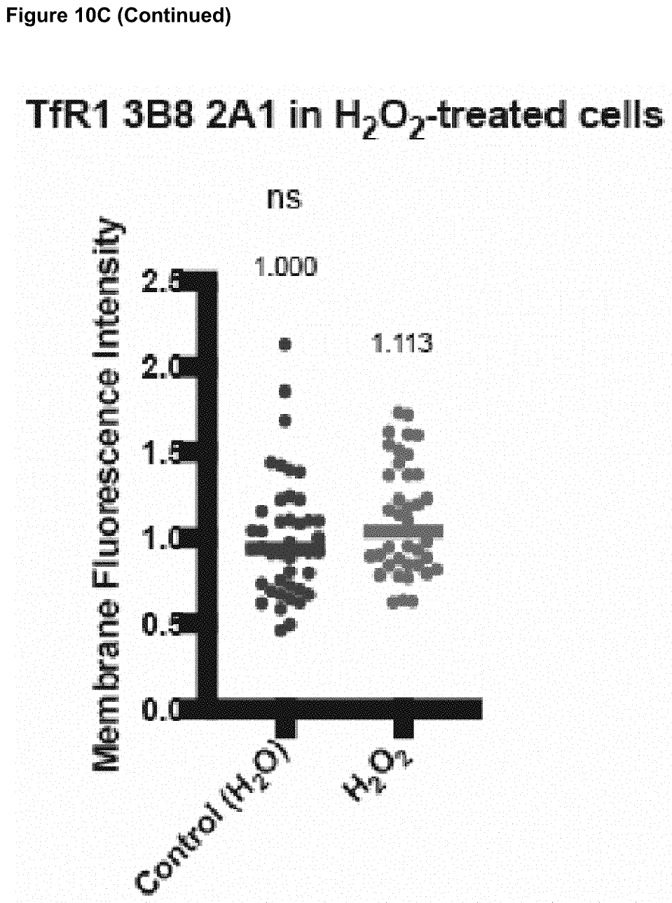

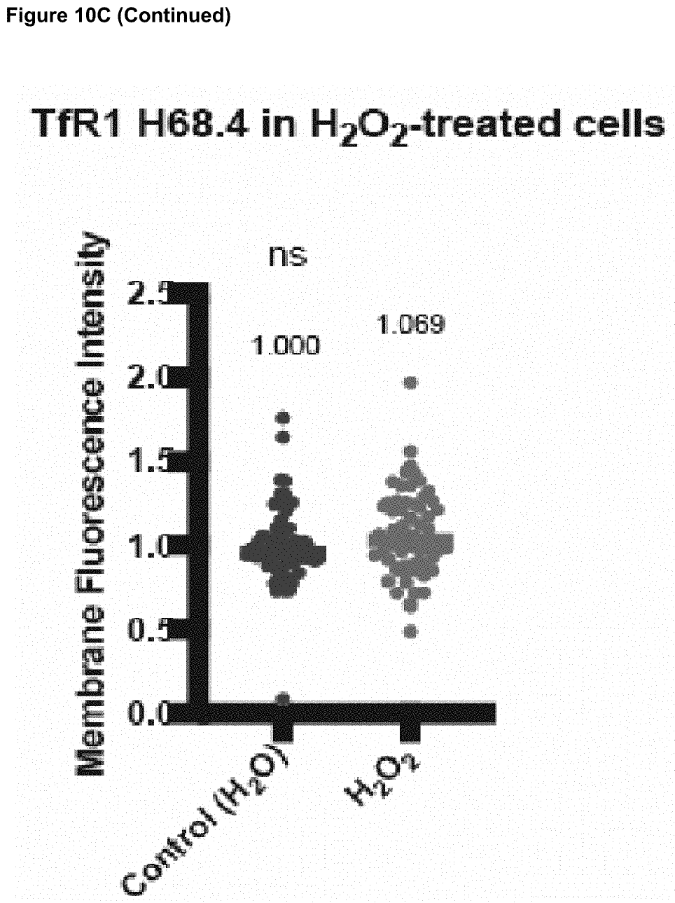

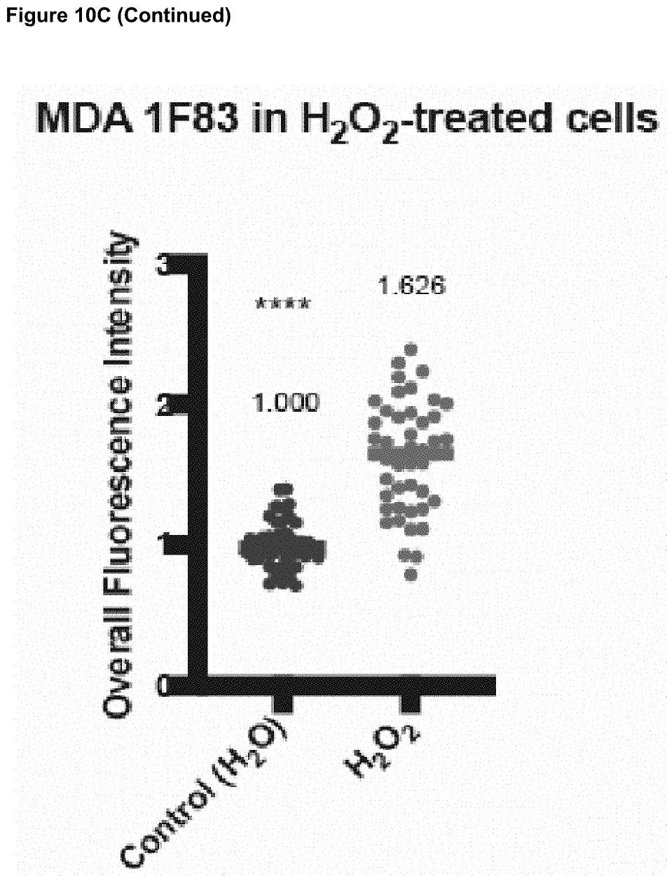

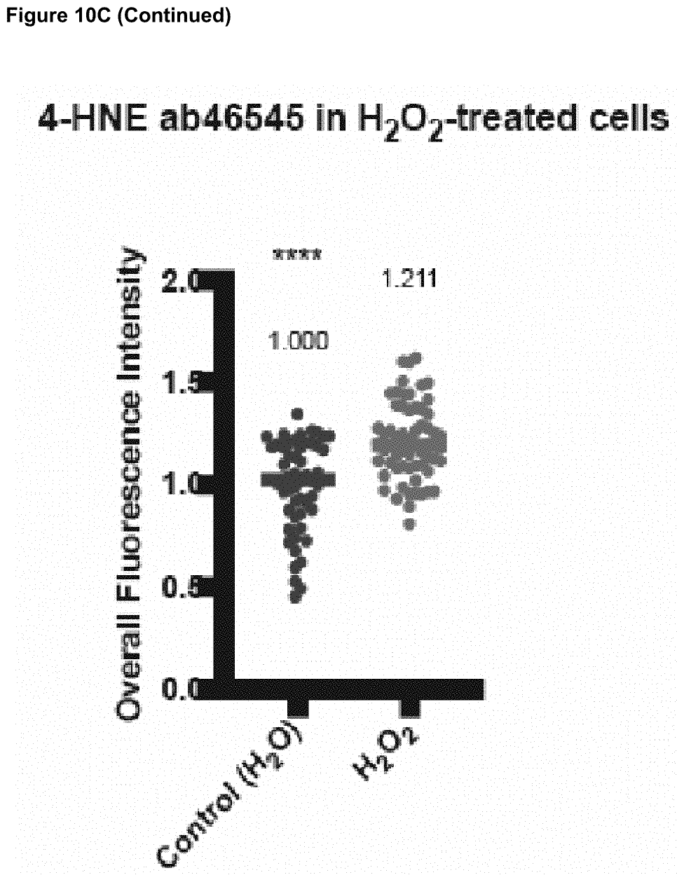

RSL3 was used to induce ferroptosis in this comparison test. We found an increase in membrane intensities for anti-TfR1 3B8 2A1 and anti-TfR1 H68.4 (FIG. 4A) but not for anti-TfR1 D7G9× (FIG. 10A). This was not unexpected, as they have different target sequences within TfR1. Both anti-TfR1 3B8 2A1 and anti-TfR1 H68.4 target ectodomains, whereas TfR1 D7G9× targets the cytoplasmic domain (residues 3-28). There was thus an increase of ectodomains of TfR1 staining as well as translocation from the cytoplasm to the cell surface during ferroptosis. 3F3-FMA might also target ectodomains, based on the similarity of the staining pattern.

Next, we compared other potential ferroptosis-staining reagents—anti-MDA 1F83, anti-MDA ab6463, anti-4-HNE ab46545 and anti-ACSL4 sc-365230 antibodies. It's found that anti-MDA 1F83 and anti-4-HNE ab46545 were capable of staining HT-1080 cells during RSL3-induced ferroptosis (FIG. 4B). We saw an increase of intensities in the cell membrane with these antibodies. However, anti-MDA ab6463 antibody and anti-ACSL4 sc-365230 antibody were not effective in these conditions (FIG. 10A). In summary, 3F3-FMA and other anti-TfR1 antibodies targeting ectodomains, anti-MDA 1F83 antibody, and anti-4-HNE ab46545 antibody can be used as ferroptosis markers by immunofluorescence in cell culture.

These antibodies were then tested for their ability to detect STS-induced apoptosis to see if these antibodies could differentiate ferroptosis from apoptosis. An anti-cleaved-caspase-3 antibody was used to verify induction of apoptosis (FIG. 2C). Unlike ferroptosis, we found that anti-TfR1 antibody staining didn't accumulate on the cell surface during apoptosis. Consistent with the formation of apoptotic bodies, TfR1 was detected outside of intact cells (FIG. 4C). Quantification did not show an increase in intensities of anti-TfR1 antibody staining in cell membranes, but rather a slight decrease. Neither membranous anti-MDA staining nor anti-4-HNE staining increased during apoptosis, indicating that both of these antibodies could differentiate ferroptosis from apoptosis (FIG. 4C).

Anti-TfR1 3B8 2A1, anti-TfR1 H68.4, anti-MDA 1F83, and anti-4-HNE ab46545 antibodies were also tested in camptothecin-induced apoptosis; cleaved PARP antibody was used to detect induction of apoptosis. We found that, as with STS-induced apoptosis, TfR1 did not accumulate on the cell surface; there was rather a decrease in membrane intensity (FIG. 10B). The membrane intensity of anti-MDA 1F83 and anti-4-HNE ab46545 antibodies didn't change in camptothecin-treated cells. This indicates that anti-TfR1 antibodies together with anti-MDA 1F83 and anti-4-HNE ab46545 are effective in differentiating ferroptosis from apoptosis.

Next, we tested whether these antibodies could differentiate between ferroptosis and more general oxidative stress that does not lead to ferroptosis. HT-1080 cells were incubated with 1 mM H₂O₂ for 4 h to test if anti-TfR1 antibodies and other potential ferroptosis-staining reagents could differentiate ferroptosis from H₂O₂-induced cell death, which has been suggested to be a necrotic death associated with oxidative stress. We didn't observe increased membrane intensities for anti-TfR1 antibodies, including 3F3-FMA, TfR1 3B8 2A1 and TfR1 H68.4 (FIG. 10C). However, we did see increased cellular intensities of anti-MDA 1F83 and anti-4-HNE ab46545 antibodies in H₂O₂-treated cells (FIG. 10C). Therefore, in the HT-1080 cell context, anti-TfR1 antibodies were able to differentiate ferroptosis from H₂O₂-induced oxidative stress and necrotic death, but anti-MDA and anti-4-HNE antibodies could not.

### Example 6

### Application of Transferrin Receptor 1 Antibodies in Flow Cytometry and Western Blot and Comparison with Other Ferroptosis-Staining Reagents

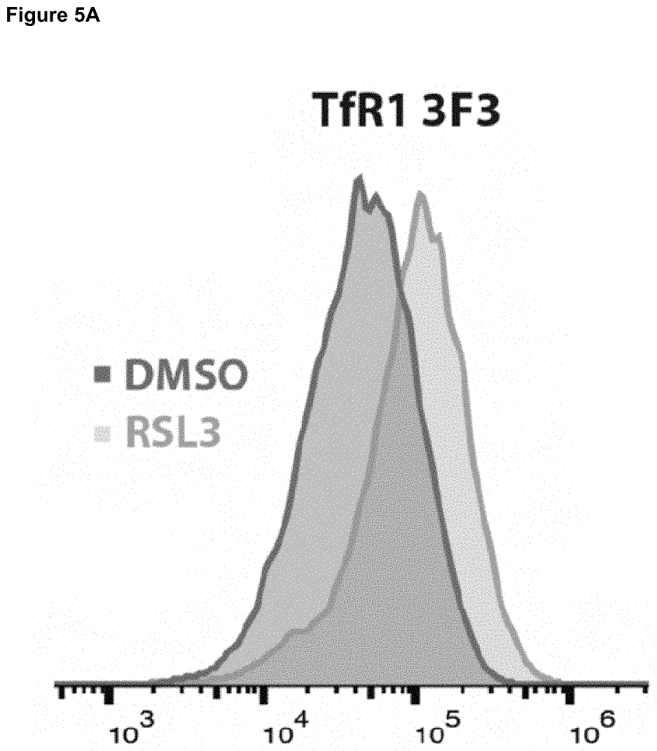

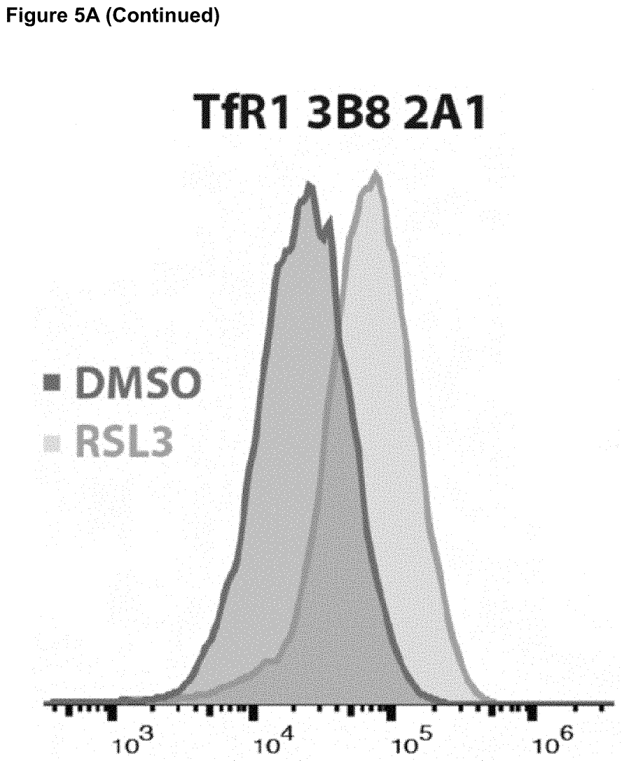

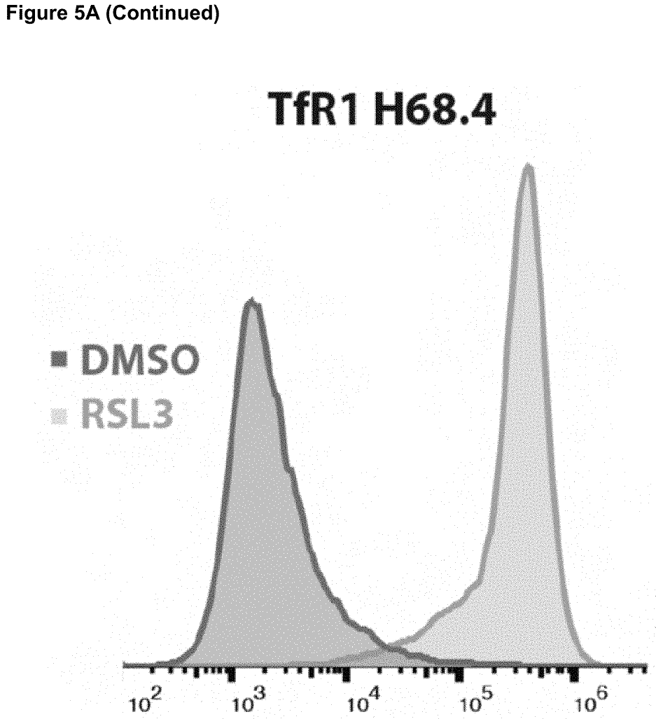

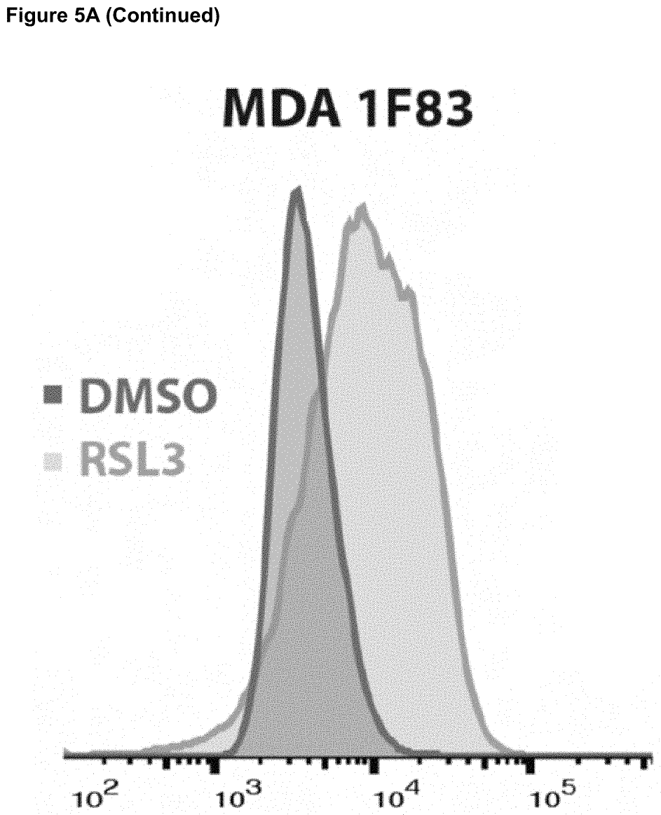

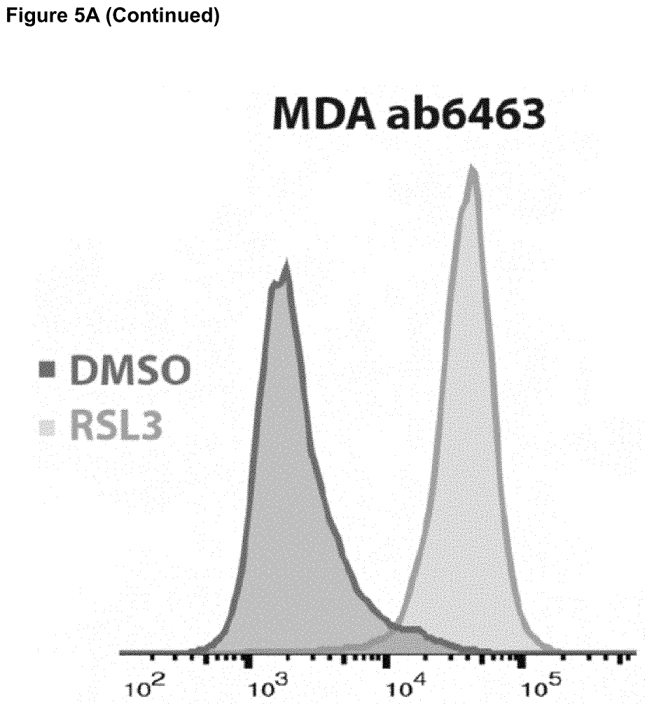

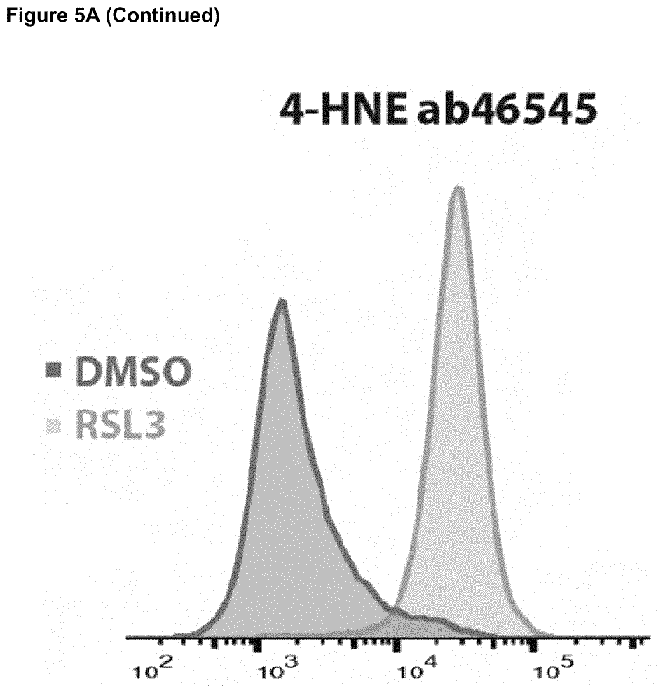

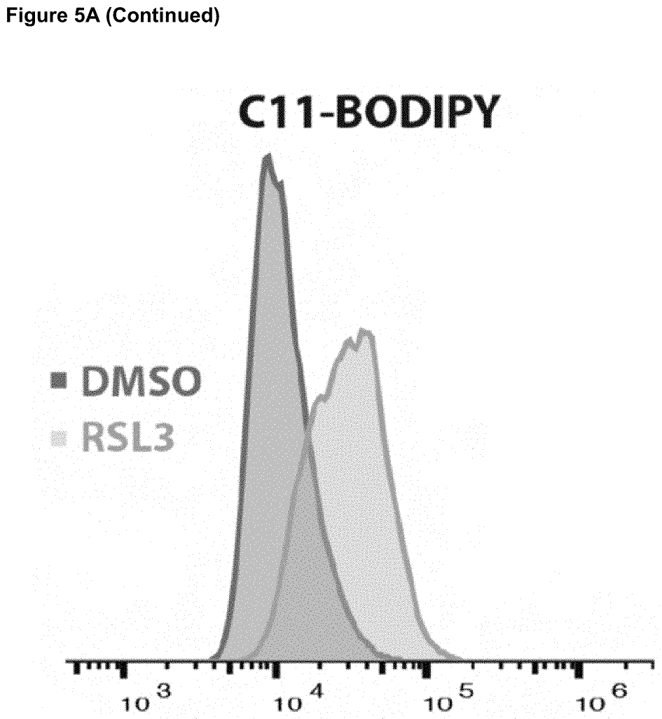

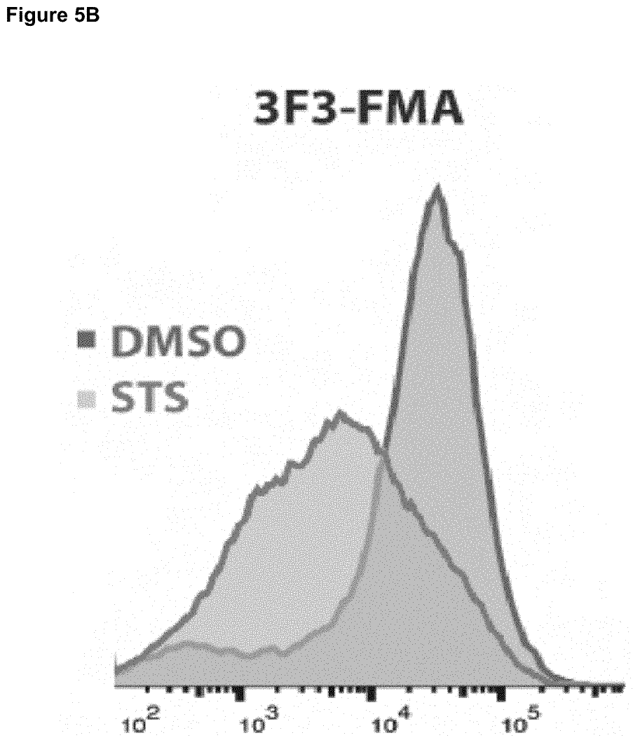

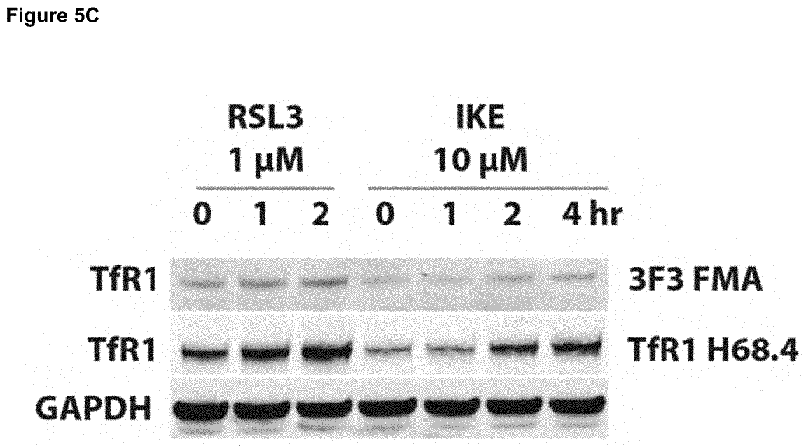

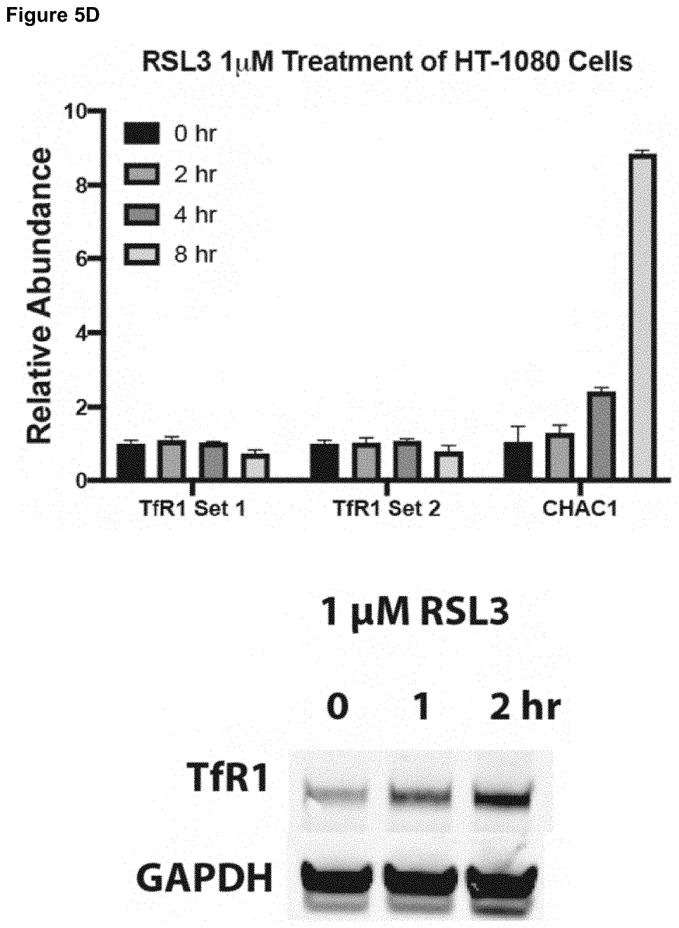

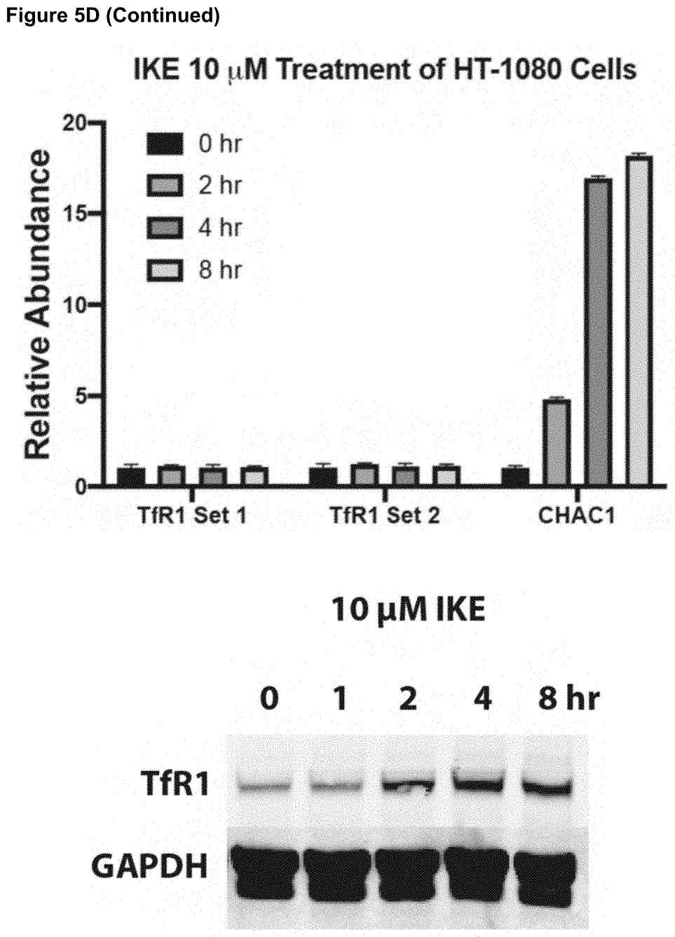

To explore the scope of applications for these antibodies, anti-TfR1, anti-MDA and anti-4-HNE antibodies were tested using flow cytometry and western blot. It's found that all of these antibodies showed increased staining intensities in RSL3-treated HT-1080 cells (FIG. 5A). Compared to C11-BODIPY, which is a sensor of lipid peroxidation, anti-TfR1 H68.4, anti-MDA ab6463 and anti-4-HNE ab46545 antibodies showed distinct differences between DMSO-treated and RSL3-treated cells. We also found that 3F3-FMA showed a decreased intensity in STS-induced apoptosis, indicating that 3F3-FMA can differentiate ferroptosis from apoptosis by flow cytometry (FIG. 5B).

In western blotting, increased intensity of the bands blotted by 3F3-FMA and anti-TfR1 H68.4 antibodies was detected in both RSL3-induced and IKE-induced ferroptosis, indicating increased level or accessibility of cellular TfR1 proteins, not just a change in localization (FIG. 5C). We tested whether the mRNA level of TfR1 changed during ferroptosis using qPCR; we didn't observe any difference, indicating that the amount of TfR1 transcript wasn't affected during ferroptosis (FIG. 5D), and that the IRP-IRE system is likely not altered during ferroptosis. We hypothesize that the increase in TfR1 protein level by western blot is due to the upregulation of translation, downregulation of proteolysis, and/or increased accessibility to the antibodies.

### Example 7

### Applications of Transferrin Receptor 1 Antibodies in Mouse Xenograft Tumor Tissues and Comparison with Other Potential Ferroptosis-Staining Reagents

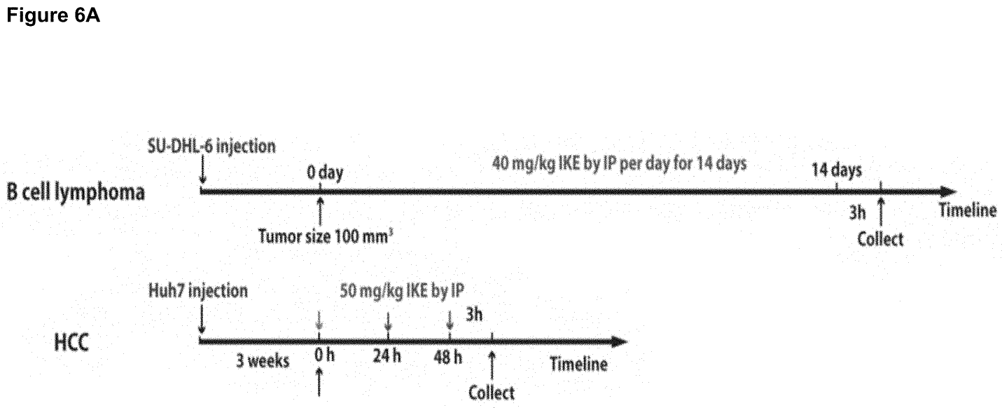

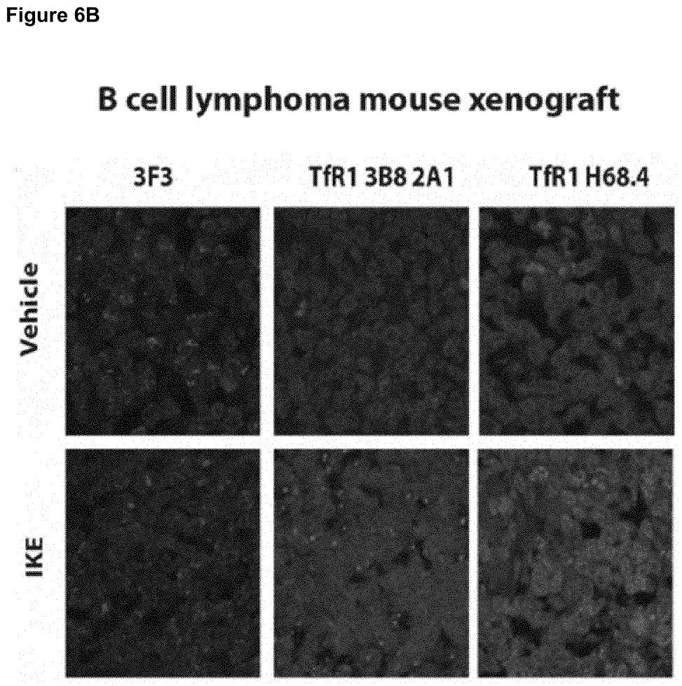

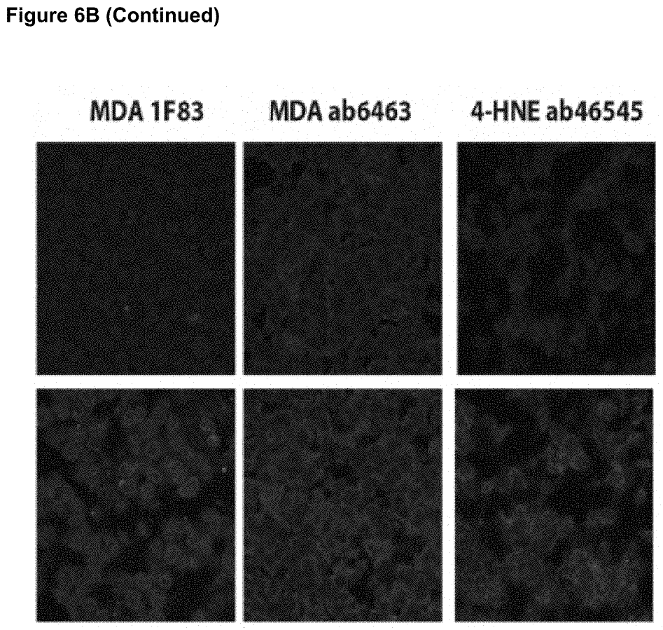

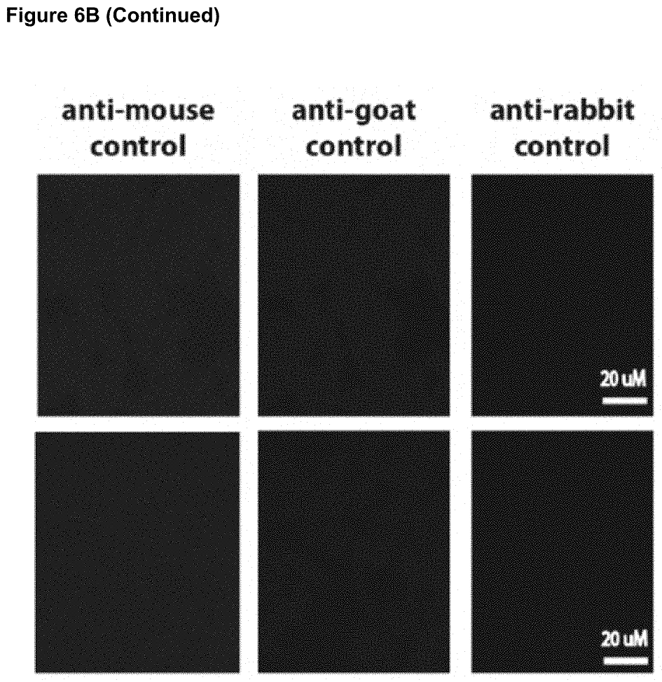

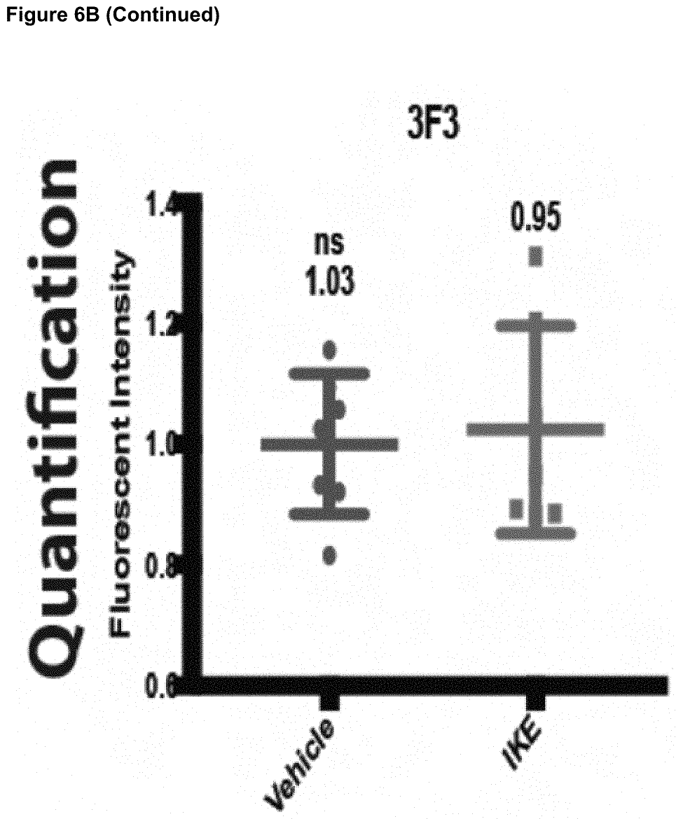

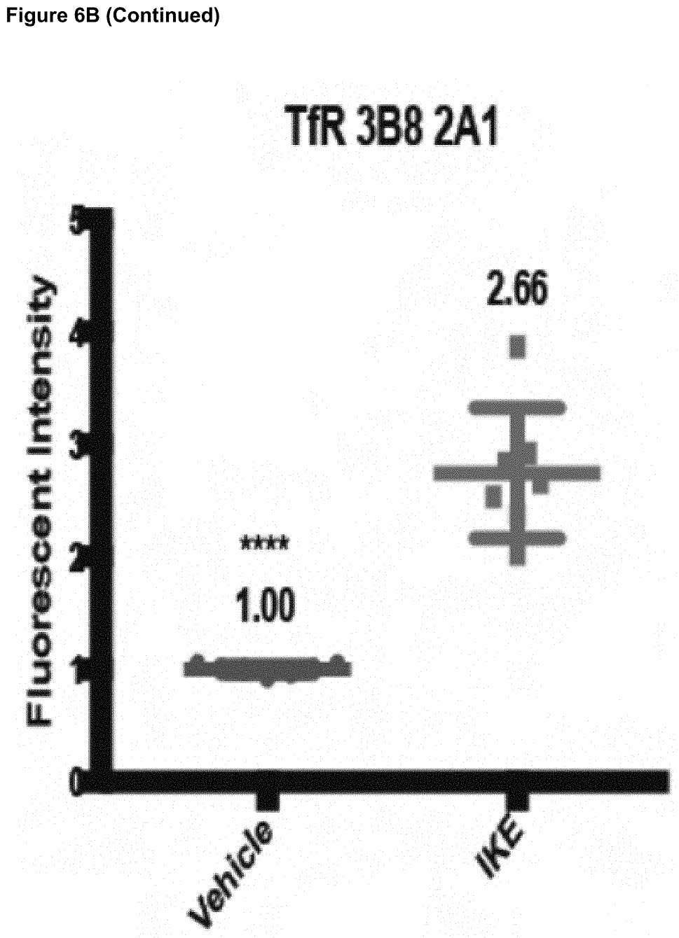

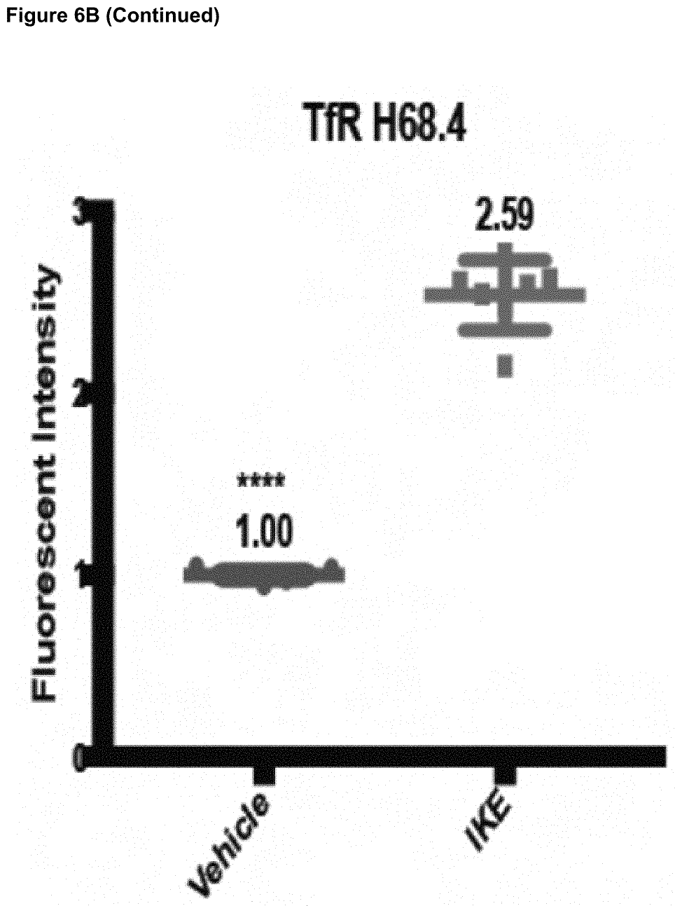

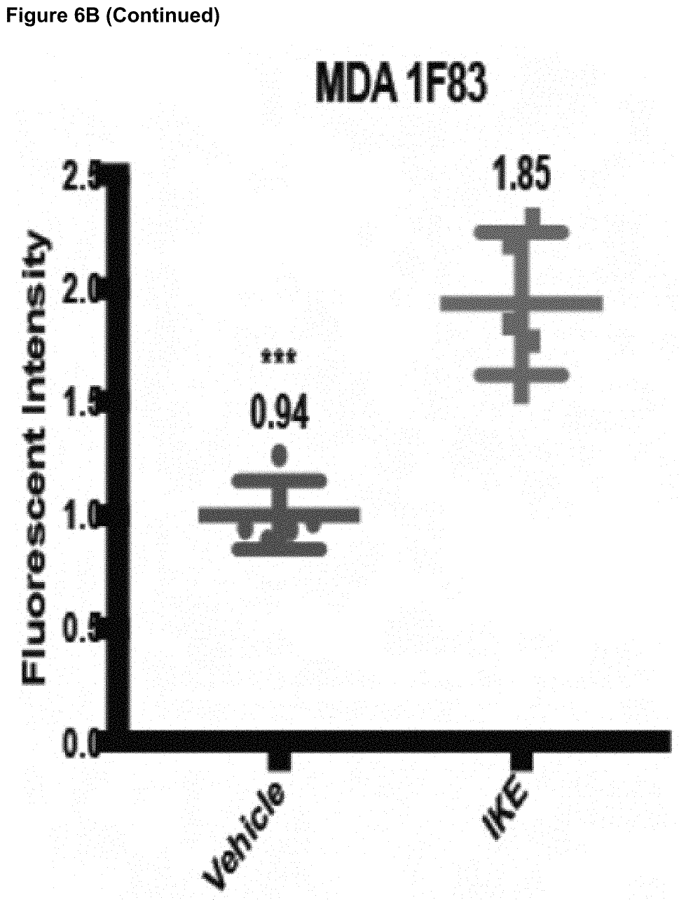

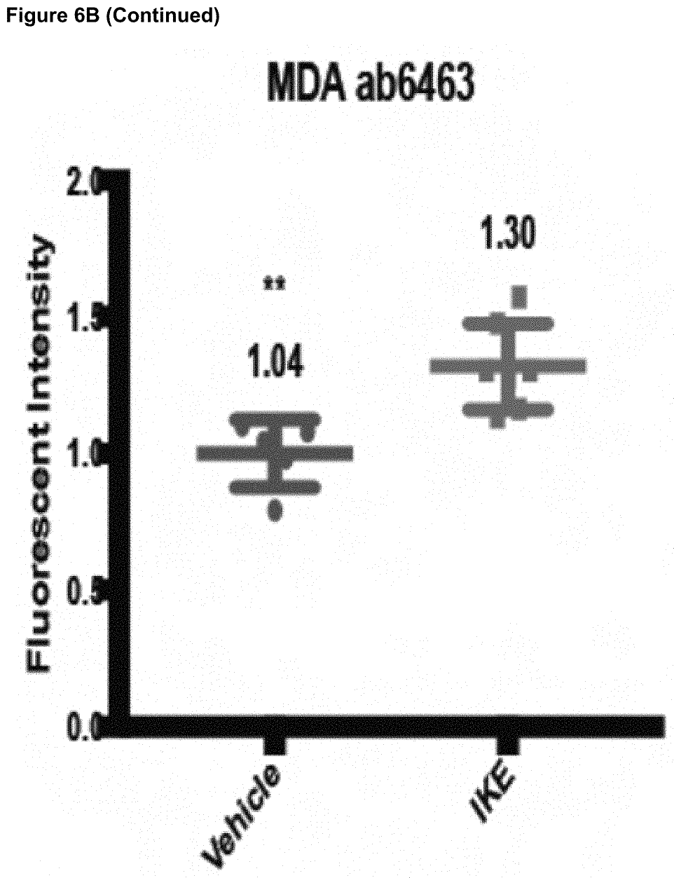

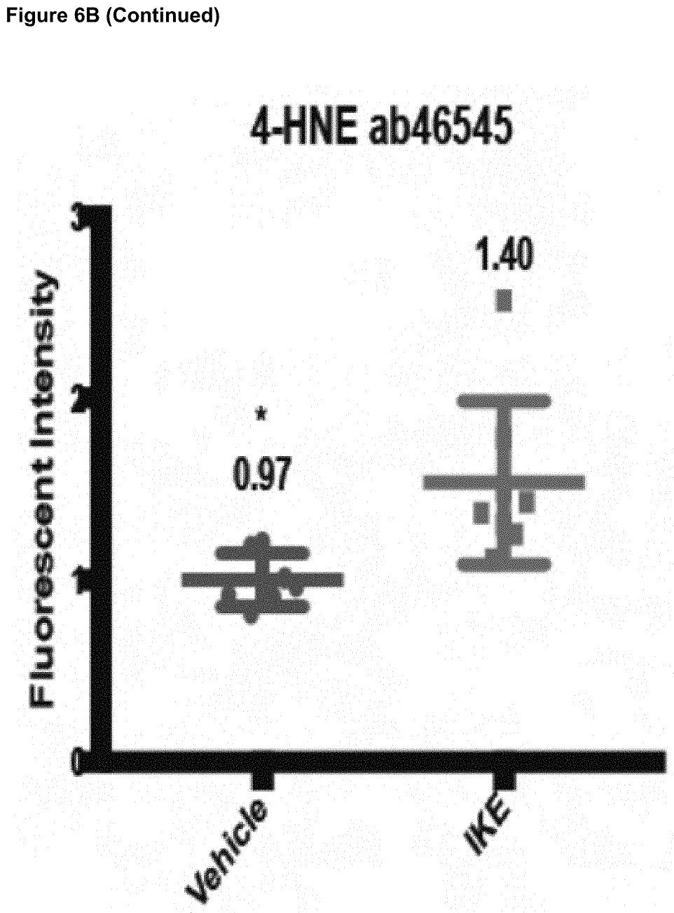

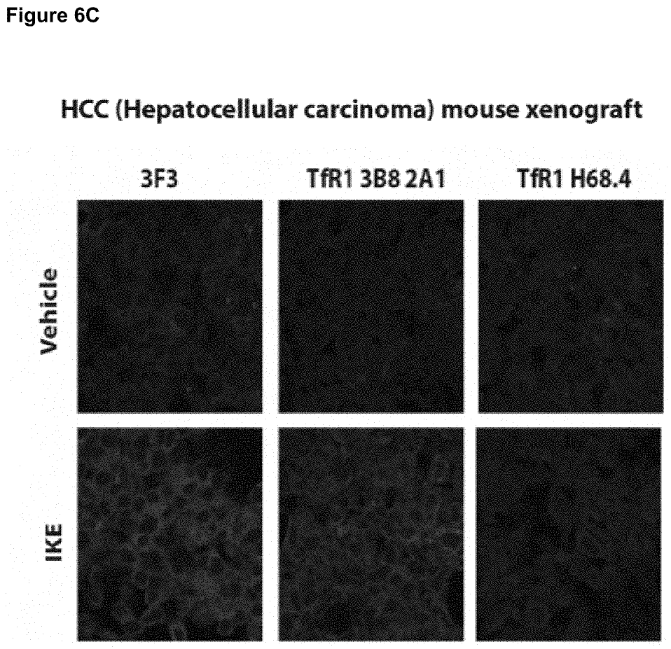

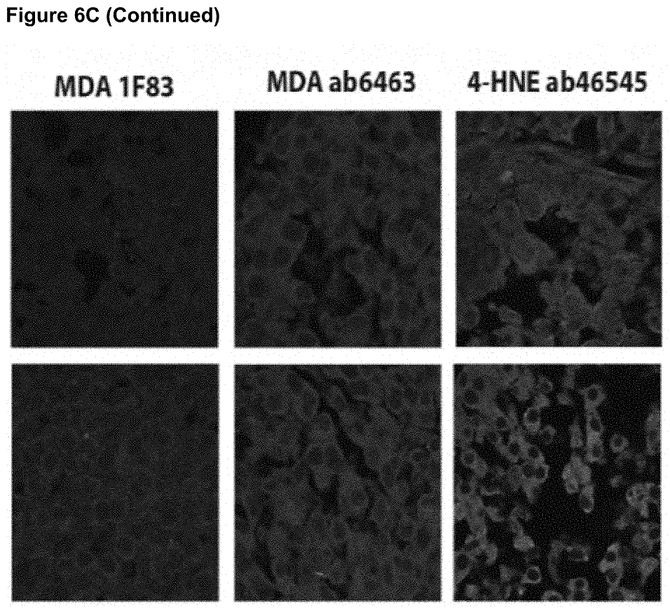

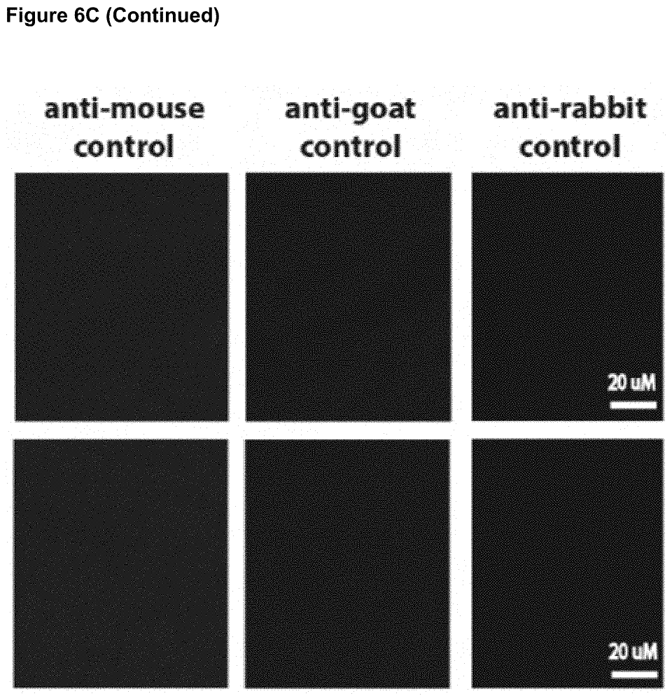

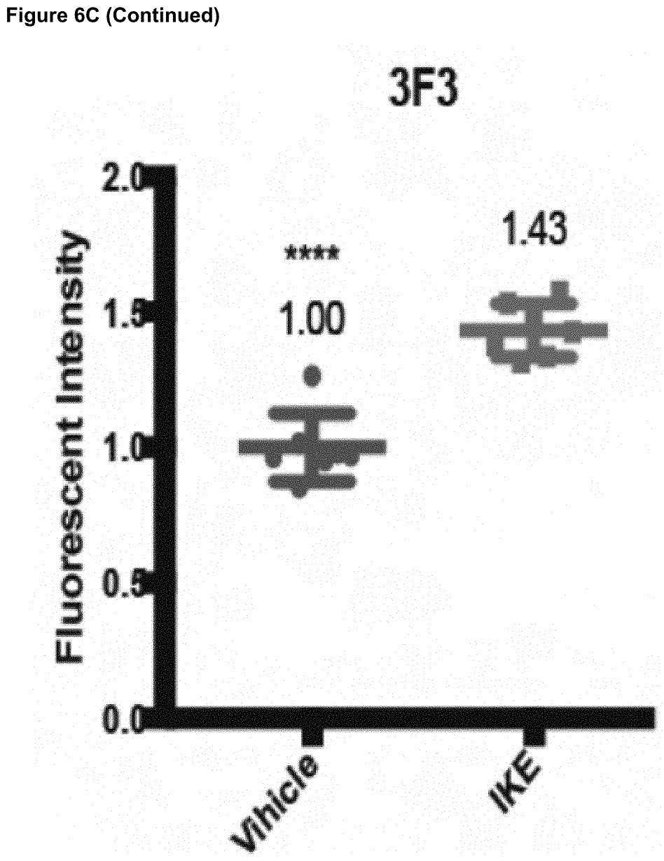

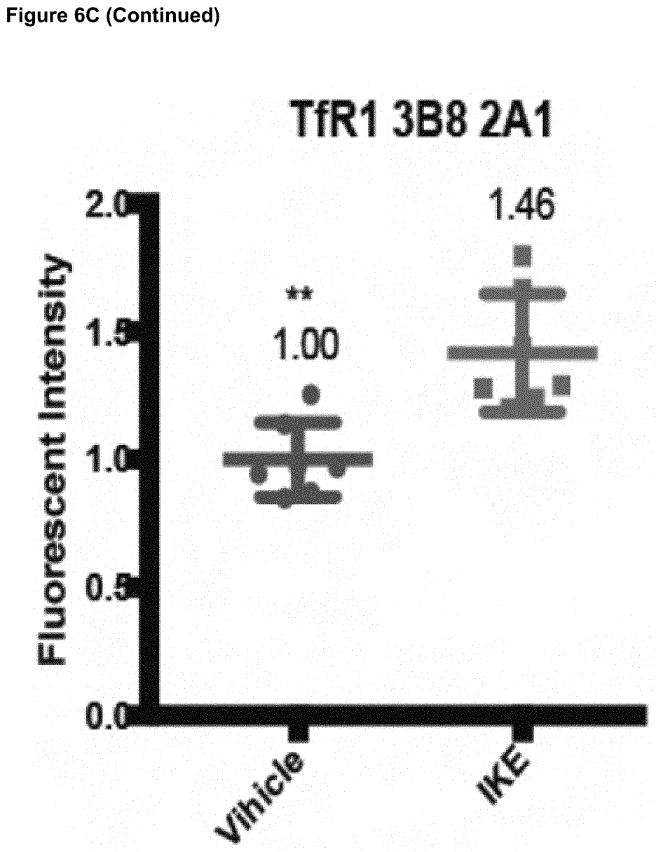

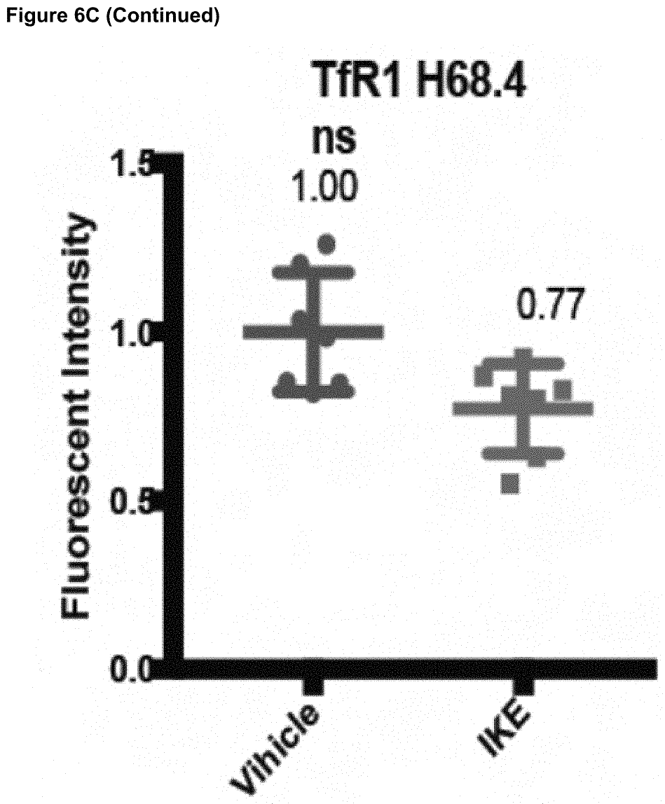

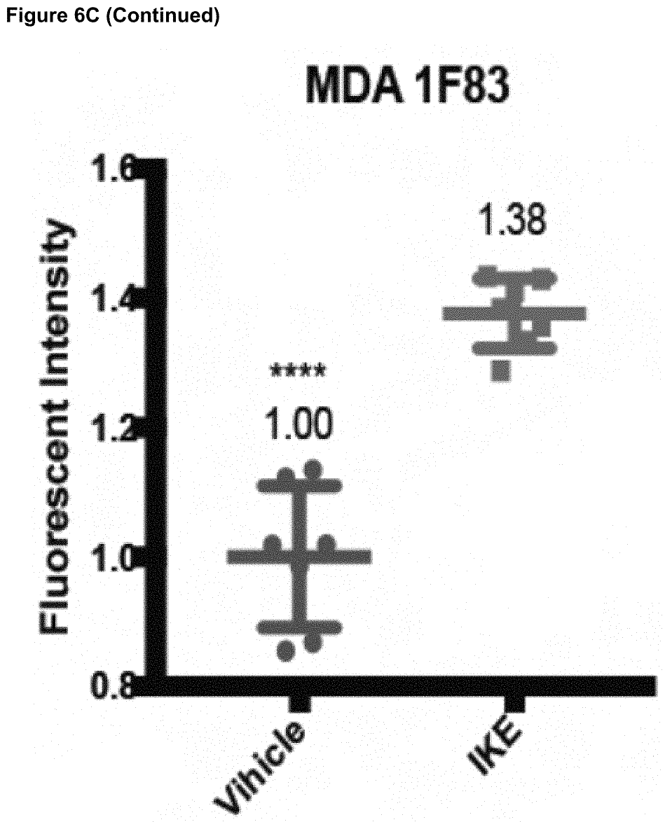

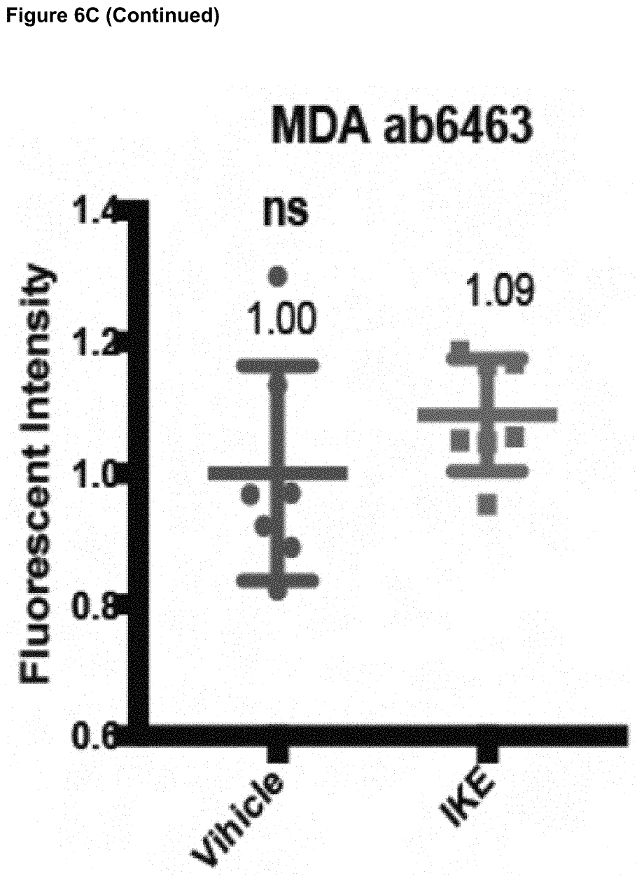

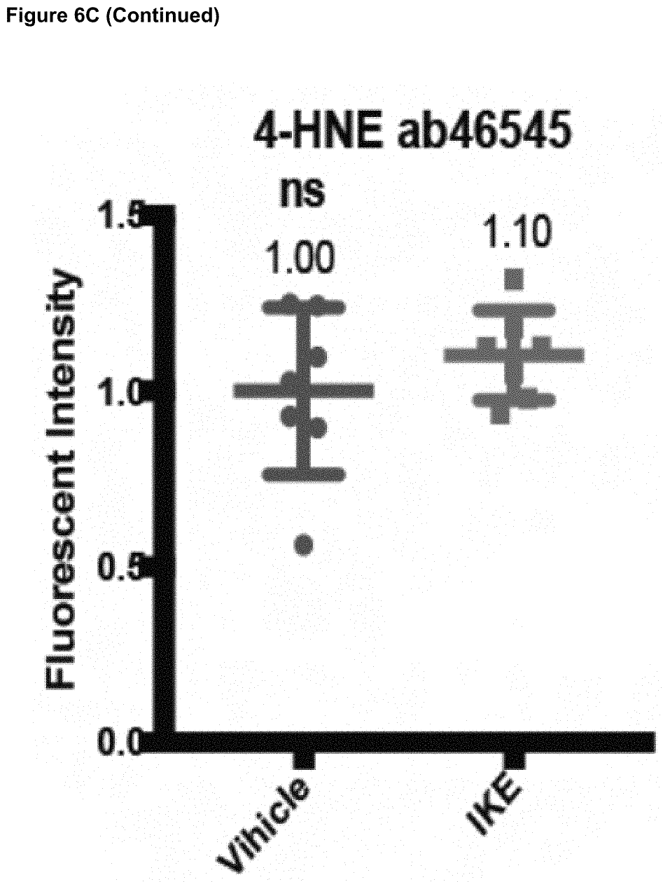

Finally, 3F3-FMA was tested together with other anti-TfR1 antibodies, as well as anti-MDA and anti-4-HNE antibodies in mouse xenograft tumor tissue sections. The preparation of human B cell lymphoma xenograft tissue samples was described previously (Zhang et al., 2019). 6-week-old NCG mice were injected with 10 million SU-DHL-6 cells subcutaneously. The mice were treated after the tumor size reached 100 mm³. Mice were separated randomly into treatment groups and dosed with vehicle and 40 mg/kg IKE once daily by IP for 14 days. 3 h after the final dosage, mice were euthanized with CO₂, and tumor tissue was dissected, frozen, fixed and cut to make slides (FIG. 6A). We found that anti-TfR1 3B8 2A1, anti-TfR1 H68.4 and anti-MDA 1F83 showed significant increase of intensities in IKE-treated samples; however, 3F3-FMA did not detect its antigen in these samples (FIG. 6B)—this may require optimization of fixation conditions for 3F3-FMA use in DLBCL xenograft tissue sections. Anti-MDA ab6463 and anti-4-HNE ab46545 showed increased intensities in IKE-treated samples as well, but to a lesser extent (FIG. 6B).

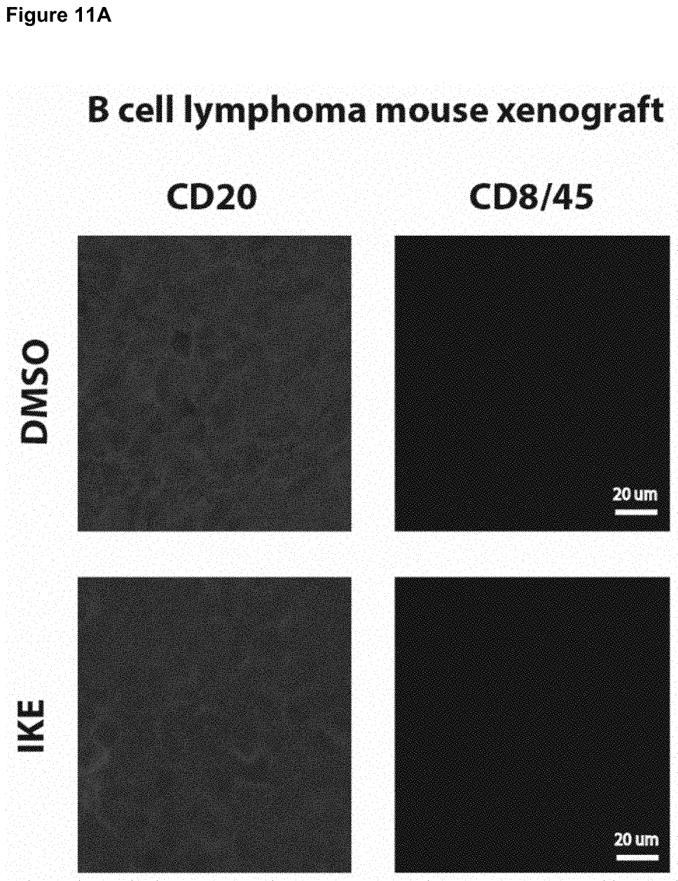

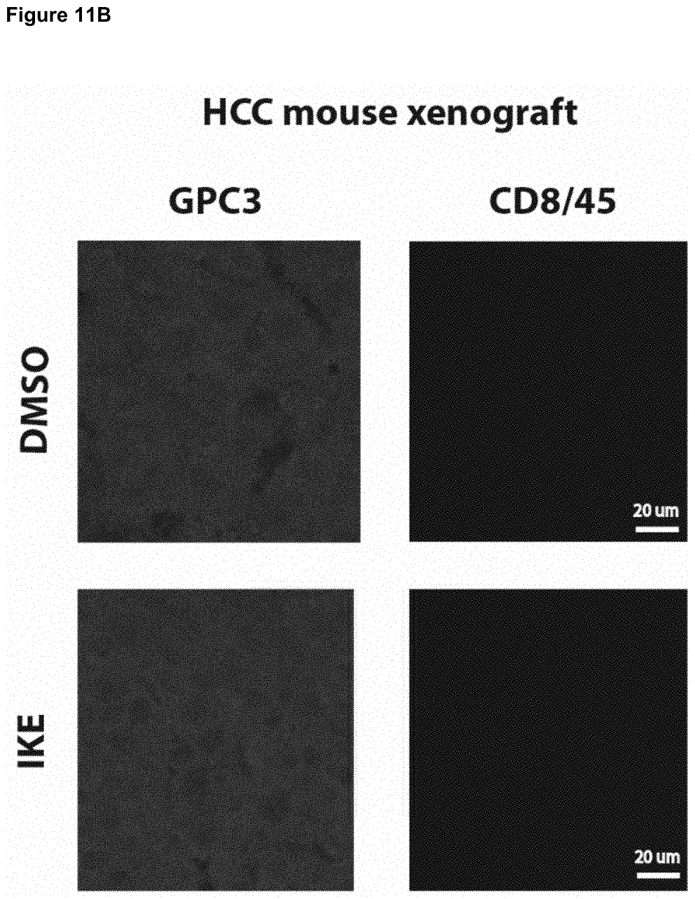

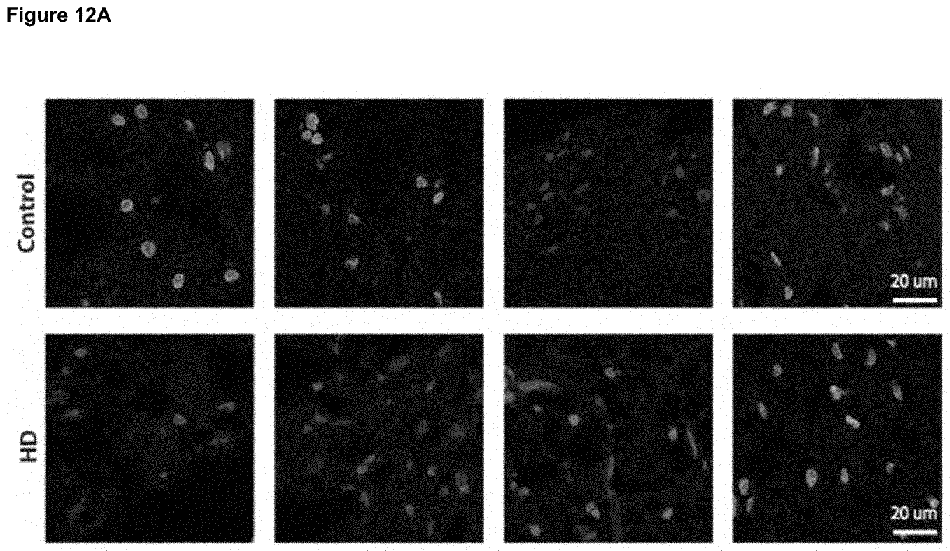

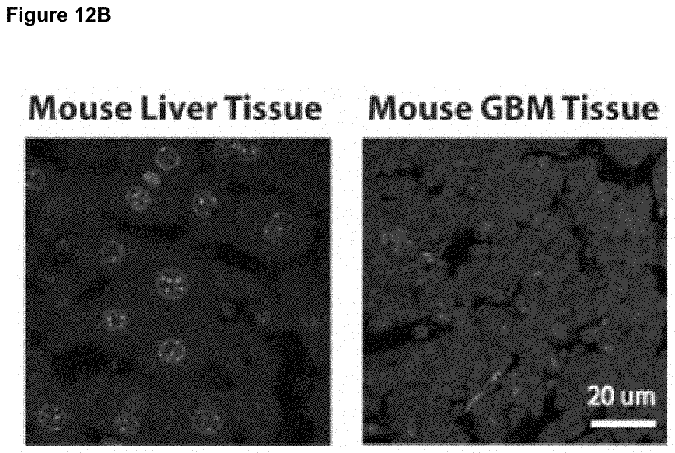

Next, HCC (Hepatocellular carcinoma) mouse xenograft tissue samples were generated by injecting 6-week-old NCG mice with 5 million human Huh-7 HCC cells. After three weeks, mice were dosed with vehicle or 50 mg/kg IKE once daily by IP for 2 days. 3 h after the final dosage, mice were euthanized with CO₂ and tumor tissue was dissected, frozen, fixed and sectioned to make slides (FIG. 6A). Only 3F3-FMA, anti-TfR1 3B8 2A1 and anti-MDA 1F83 antibodies showed increased intensities in IKE-treated samples (FIG. 6C). We validated that tumor cells, but not infiltrating immune cells, were stained in both of these mouse xenograft tissue samples using the cell markers CD20, CD8/45 and GPC3 (FIGS. 11A-11B). Overall, anti-TfR1 3B8 2A1 and anti-MDA 1F83 showed the strongest increases in both samples and are recommended as robust ferroptosis markers for frozen tissue xenograft samples. We were also interested in determining whether 3F3-FMA could be used to detect TfR1 in normal tissues. We evaluated 3F3-FMA staining in post-mortem human brain tissues: we compared the level of staining in Huntington's disease (HD) and control human brain tissues. The expression of TfR1 in brain tissue was apparently low, as evidenced by a lack of signal, and we did not detect any differences between the control group and HD group (FIG. 12A). We also evaluated 3F3-FMA in normal mouse liver frozen tissue samples. We detected a robust signal for 3F3-FMA in this tissue. This suggests that human TfR1 expression may be low in normal human brain tissue and higher in normal mouse liver, such that it may be feasible to detect ferroptosis in human brains and mouse livers in some disease contexts, if TfR1 abundance increases substantially in disease contexts.

Finally, we sought to test whether 3F3-FMA could be used in paraffin-embedded tissue samples, which are historically more abundant and accessible than fresh frozen tissues. We evaluated 3F3-FMA staining in mouse glioblastoma (GBM) paraffin-embedded tissue samples, to see if the increased TfR1 that has been observed in many tumors would render 3F3-FMA staining detectable in this setting over the low signal evident in normal brain tissue. 3F3-FMA was indeed able to recognize mouse TfR1 protein in these tumor samples, suggesting future studies could evaluate TfR1 levels as a ferroptosis marker in mouse, and possibly human, glioblastoma samples (FIG. 12B).

### Example 8

### Discussion

3F3-FMA was assessed together with three commercially available anti-TfR1 antibodies and four additional potential ferroptosis-staining reagents in different assays (immunofluorescence, flow cytometry, and tissue sections). A summary of applications is shown in Table 1. The anti-TfR1 3B8 2A1 and anti-MDA 1F83 antibodies perform best. They yielded reliable results in mouse xenograft tumor tissue samples, as well as immunofluorescence and flow cytometry applications. We propose that researchers can use a combination of anti-TfR1 3B8 2A1 and anti-MDA 1F83 antibodies as ferroptosis markers to stain human tissue sections, which will aid research on the role of ferroptosis in human disease.

The identification of TfR1 accumulation on the cell surface as a feature of ferroptosis is significant. Nonetheless, specificity is a potential limitation of using anti-TfR1 antibodies as ferroptosis markers. We found, however, that anti-TfR1 antibodies could differentiate ferroptosis from apoptosis; other cell death forms including necroptosis, autophagic death and pyroptosis have not been tested. It was reported previously that uptake of extracellular iron by a TfR1-dependent iron transport mechanism was required in hydroperoxide-induced DCFH oxidation and endothelial cell apoptosis (Tampo et al., 2003). Treatment with an anti-TfR1 antibody also dramatically inhibited iron uptake, intracellular oxidant formation, and doxorubicin-induced apoptosis (Kotamraju et al., 2002). Further validation of the specificity of anti-TfR1 antibodies is therefore needed across diverse contexts before we can be certain of its suitability as a specific marker of ferroptosis.

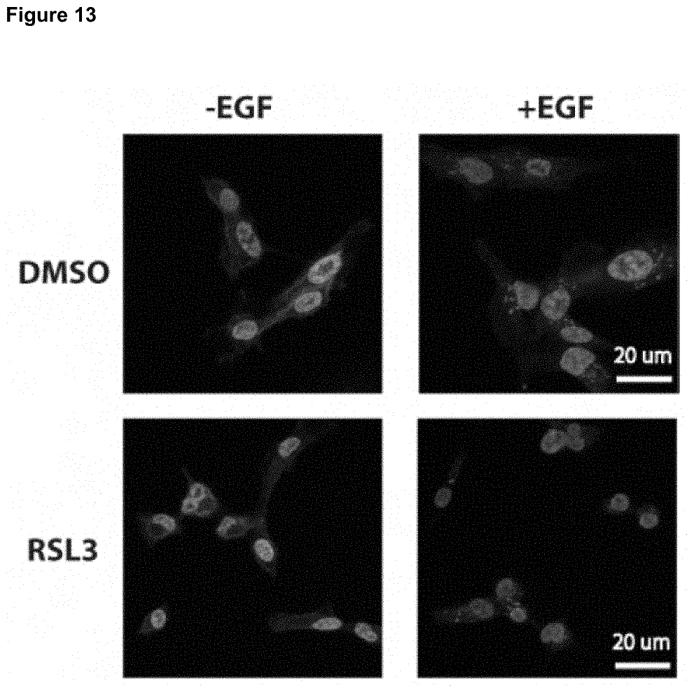

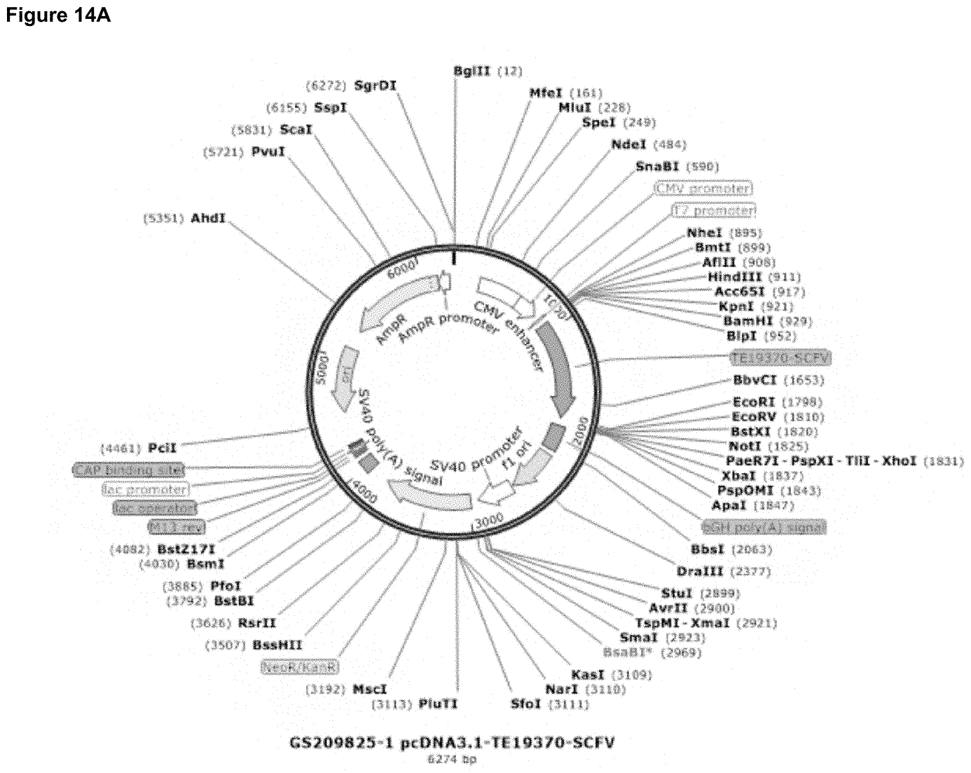

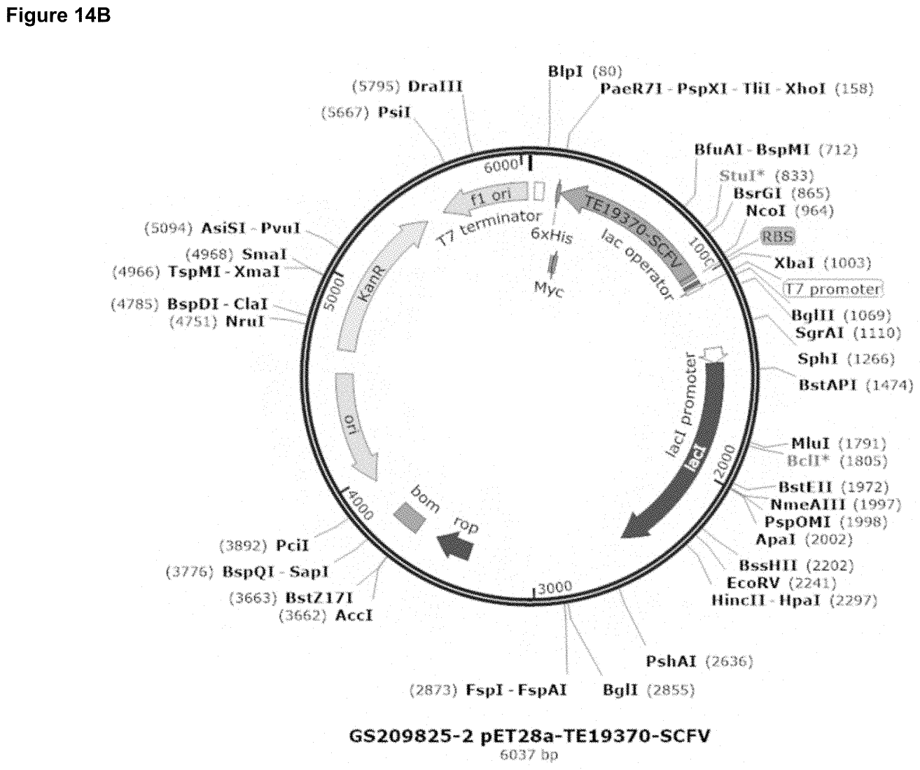

In addition to its potential use as a ferroptosis marker, future studies may examine why TfR1 accumulation on the cell surface occurs during ferroptosis. One hypothesis is that the internalization machinery is disrupted in the ferroptotic context. To test this hypothesis, we examined Epidermal Growth Factor Receptor (EGFR) because it uses the same clathrin-mediated endocytosis with TfR1 and is internalized in the presence of EGF only. However, EGFR was still internalized in the presence of EGF during ferroptosis, indicating that clathrin-mediated endocytosis is not affected during ferroptosis (FIG. 13). Therefore, we hypothesize that recruitment of TfR1 to the plasma membrane during ferroptosis is related to iron metabolism. This might occur through a positive feedback cycle between iron uptake and ferroptotic death.

A further direction for the future is the study of the role of TfR1 in ferroptosis. It was reported previously that cells with knockdown of TfR1 became more resistant to erastin-induced cell death (Yang and Stockwell, 2008) and that siTfR1 RNAi significantly inhibited serum-dependent necrosis, which was subsequently determined to be ferroptosis (Gao et al., 2015). These results indicate that decreased iron uptake caused by knockdown of TfR1 is implicated in ferroptosis. However, the cellular iron pool is also controlled by an iron storage protein complex consisting of ferritin heavy chain 1 (FTH1) and ferritin light chain (FTL) (Harrison and Arosio, 1996). The precise role of TfR1 and the related iron metabolism pathway in ferroptosis remains to be determined.

TfR1 is abundantly expressed and actively involved in the progression of several kinds of cancers, including brain cancer, breast cancer, colon cancer, and liver cancer, rendering TfR1 a valuable target (Daniels et al., 2012). The increased need for iron uptake leads to the high expression of TfR1, because iron is required for tumor cell proliferation (Marques et al., 2016). On the other hand, the upregulation of iron uptake by TfR1 also refills the labile redox-active iron pool, which is needed for ferroptosis. Therefore, how iron metabolism is regulated between tumor progression and ferroptotic tumor suppression remains elusive. More research is needed to define the relationship between TfR1 expression, iron metabolism, ferroptosis and cancer progression.

In summary, we began with a pool of antibodies with unknown targets generated from PE-treated cell membrane fractions. The 3F3-FMA antibody was selected to mark ferroptotic cells and its antigen was identified as transferrin receptor protein 1 (TfR1). Different anti-TfR1 antibodies and other potential ferroptosis staining reagents were assessed in immunofluorescence, flow cytometry, western blot and tissue samples. Anti-TfR1 3B8 2A1 and Anti-MDA 1F83 antibodies were selected as the best-performed reagents across immunofluorescence, flow cytometry and tissue section applications. We recommend using a combination of these two antibodies to detect cells undergoing ferroptosis in diverse contexts.

TABLE 1
A summary of different applications for all antibodies

| | antibody | | | | | | | |
|---|---|---|---|---|---|---|---|---|
| | | TfR1 | | | | | | |
| | | 3B8 | TfR1 | TfR1 | MDA | MDA | 4-HNE | ACSL4 |
| application | 3F3 | 2A1 | H68.4 | D7G9X | 1F83 | ab6463 | ab46545 | sc-365230 |
| IF | [symbol] | [symbol] | [symbol] | [symbol] | [symbol] | [symbol] | [symbol] | [symbol] |
| Flow Cytometry | [symbol] | [symbol] | [symbol] | NA | [symbol] | [symbol] | [symbol] | NA |
| Lymphoma Tissue | [symbol] | [symbol] | [symbol] | NA | [symbol] | [symbol] | [symbol] | NA |
| HCC Tissue | [symbol] | [symbol] | [symbol] | NA | [symbol] | [symbol] | [symbol] | NA |

Eight antibodies were evaluated in IF (immunofluorescence), flow cytometry and two mouse xenograft tumor tissue samples using immunofluorescence. Anti-TfR1 3B8 2A1 and anti-MDA 1F83 performed best overall.

### Example 9

### Vector Constructions for Expressing 3F3-FMA in scFv Format

Further, the following vectors were constructed to express 3F3-FMA in the form of scFv in both mammalian cell and bacteria,

pcDNA3.1-scFv Construction (for Mammalian Cell Expression)

A target gene scFv of 875 bp was synthesized (5′-GGATCCGCCGCCACCATGCACAGCTCAGCACTGCTCTGTTGCCTGGTCCTCCTGA CTGGGGTGAGGGCCGAAGTCCTCCTCCAACAATCCGGAACAGAACTCGTTAGACC TGGTGCACTGGTCAAGCTCTCCTGTAAAGCCAGTGGATTCAACATTCAGGACCTCT ACATTCACTGGGTCAAGCAACGCCCAGAGCAAGGGCTGGAGTGGATCGGCTGGA TTGATCCCGAGACAGATAACACAATCTACGATCCTAAGTTCCAGGGTAAGGCATCC ATAACTGCCGACACAAGCAGTAATACTGCATACCTCCAGCTGTCATCACTCACCAG CGAGGATACAGCCATGTACTACTGTTCCACTGGACTGCTGCAATGGTACTTTGATG TTTGGGGAGCAGGGACTGCCGTTACCGTGTCCTCTGGAGGCGGAGGGTCCGGTG GAGGAGGCTCCGGAGGTGGTGGCAGCGACATAGTCATGACACAGAGCCCAAGCA GCCTGGCTATGAGCATCGGGCAGAAGGTGACAATGCGGTGCAAGTCCTCTCAGAG TCTGCTGAACTCCTACAATCAGAAGAATTGTCTGGCCTGGTATCAACAGAAGCCAG GCCAAAGTCCTAAGCTCCTCCTGTATTTCGTTTCAACTCGCGAGTCTGGTGTGCCT GACAGATTCATCGGCAGTGGGAGCGGGACAGACTTCACTCTCACCATCAGTAGTG TCCAGGCTGAGGACCTGGCCGACTATTTCTGTCAACAACATTACTCAACTCCTCTG ACTTTCGGTGCTGGAACCAAACTGGAGCTGAAGGGCAGCGAGCAGAAACTGATTT CCGAAGAGGACCTGAGCCACCATCACCACCATCACTAATAGAATTC-3′, SEQ ID No: 22). The plasmid vector pcDNA3.1 and target gene were digested by EcoRI and BamHI. The digestion reaction was performed in a 37° C. water bath for 2 hours. The plasmid pcDNA3.1 and target gene were recovered by 1% agarose gel electrophoresis. The recovered plasmid pcDNA3.1 were ligated to the recovered target gene, and the ligation reaction was performed at 16° C. for 12 hours. Took 10 μl ligation product and 100 μl DH5a competent bacteria and mixed in an ice bath for 30 min, heat shock at 42° C. for 90 s, immediately placed on ice for 5 min, then added 700 μl LB medium, incubated at 37° C. for 50 min, and sucked 200 μl of the bacterial solution. After mixing with a pipette, evenly spreaded on a LB plate containing 100 μg/ml Ampicillin, and cultured in a 37° C. incubator overnight. Picked 5 single colonies and inoculated them in 5 ml LB culture medium containing 100 μg/ml Ampicillin, cultured at 300 rpm, 37° C. in a constant temperature shaker overnight, and expanded the overnight bacterial solution. The plasmid was extracted by the quantitative extraction kit, and then verified by sequencing.

pET28a-scFv Construction (for Bacterial Expression)

A target gene scFv of 812 bp was synthesized (5′-CCATGGAAGTGCTGCTGCAACAAAGCGGCACCGAACTGGTACGTCCGGGTGCTCT GGTGAAACTGTCTTGTAAAGCATCTGGTTTCAACATCCAGGACCTGTACATTCACT GGGTTAAACAGCGTCCGGAACAAGGCCTGGAATGGATCGGTTGGATCGACCCGG AAACTGACAACACTATCTACGACCCGAAATTCCAAGGTAAAGCAAGCATTACCGCA GACACCTCTTCCAACACCGCGTACCTGCAACTGTCCTCTCTGACCTCTGAAGATAC CGCAATGTACTACTGTAGCACTGGTCTGCTGCAATGGTACTTTGATGTTTGGGGTG CCGGTACTGCGGTGACTGTTTCCTCTGGTGGCGGTGGTTCTGGCGGTGGTGGTTC TGGTGGTGGTGGCTCTGACATTGTGATGACCCAGTCTCCGAGCAGCCTGGCGATG TCCATCGGTCAGAAGGTTACTATGCGCTGCAAGTCCTCCCAGTCCCTGCTGAACTC CTATAACCAGAAGAATTGTCTGGCTTGGTATCAGCAGAAACCGGGTCAATCTCCGA AGCTGCTGCTGTACTTTGTTTCTACTCGTGAGTCCGGTGTACCAGATCGCTTTATC GGTTCCGGTTCTGGCACCGACTTCACCCTGACCATCAGCTCCGTTCAGGCGGAGG ATCTGGCCGACTATTTCTGCCAGCAACACTATAGCACTCCGCTGACCTTTGGTGCT GGCACCAAACTGGAACTGAAGGGTTCTGAGCAGAAACTGATTAGCGAAGAGGATC TGTCTCATCACCACCACCATCATTAATAGCTCGAG-3′, SEQ ID No: 23). The plasmid vector pET28a and target gene were digested by NcoI and XhoI, and the digestion reaction was performed in a 37° C. water bath for 2 hours. The plasmid pET28a and target gene were recovered by 1% agarose gel electrophoresis. The recovered plasmid pET28a were ligated to the recovered target gene, and the ligation reaction was performed at 16° C. for 12 hours. Took 10 μl ligation product and 100 μl DH5a competent bacteria and mixed in an ice bath for 30 min, heat shock at 42° C. for 90 s, immediately placed on ice for 5 min, then add 700 μl LB medium, incubated at 37° C. for 50 min, and sucked 200 μl of the bacterial solution. After mixing with a pipette, evenly spreaded on a LB plate containing 100 μg/ml Kanamycin, and cultured in a 37° C. incubator overnight. Picked 5 single colonies and inoculated them in 5 ml LB culture medium containing 100 μg/ml Kanamycin, cultured at 300 rpm, 37° C. in a constant temperature shaker overnight, and expanded the overnight bacterial solution. The plasmid was extracted by the quantitative extraction kit, and then verified by sequencing.

### Example 10

### Applications of Transferrin Receptor 1 Antibodies in Combination Therapy of Ferroptosis Inducers and Radiation

We previously demonstrated that small molecule inducers of ferroptosis could synergize with radiation to promote cancer cell killing. Using a combination of radiation and IKE or sorafenib, we showed that synergistic tumor cell killing through ferroptosis can be extended to patient-derived models of glioma and lung cancer.

To improve/maximize the efficiency of the combination therapy of ferroptosis inducers and radiation, we will introduce the transferrin receptor 1 antibodies disclosed herein to the combination treatment in order to obtain critical information including, for example, whether the subject is undergoing ferroptosis, whether the treatment protocol needs adjustment such as, for example, modifying the dosage of the ferroptosis inducer, or replacing the ferroptosis inducer for another, etc.

### Example 11

### Machine Learning Classifies Ferroptosis and Apoptosis Cell Death Modalities with TfR1 Immunostaining

Determining cell death mechanisms occurring in patient and animal tissues is a longstanding goal that requires suitable biomarkers and accurate quantification. However, effective methods remain elusive. To develop more powerful and unbiased analytic frameworks, we developed a machine learning approach for automated cell death classification. Image sets were collected of HT-1080 fibrosarcoma cells undergoing ferroptosis or apoptosis and stained with an anti-transferrin receptor 1 (TfR1) antibody, together with nuclear and F-actin staining. Features were extracted using high-content-analysis software, and a classifier was constructed by fitting a multinomial logistic lasso regression model to the data. The prediction accuracy of the classifier within three classes (control, ferroptosis, apoptosis) was 93%. Thus, TfR1 staining, combined with nuclear and F-actin staining, can reliably detect both apoptotic and ferroptosis cells when cell features are analyzed in an unbiased manner using machine learning, providing a method for unbiased analysis of modes of cell death.

### INTRODUCTION

Regulated cell death is a complex and tightly regulated phenomenon, involving intricate molecular mechanisms. For numerous cell death processes, molecular markers have been developed that identify cells undergoing apoptosis (Denton and Kumar, 2015) or necroptosis (He et al. 2022) through immunolabeling. Such markers may be used in cell culture and tissue histopathological applications to examine the prevalence of cell death processes, which may improve the treatment and diagnosis of diseases in which these processes are implicated.

Ferroptosis is a form of regulated cell death characterized by the iron-dependent accumulation of lipid peroxides, as well as the loss of cellular antioxidant repair capabilities (Stockwell et al. 2017). The enzyme glutathione peroxidase 4 (GPX4) is a cellular regulator of lipid peroxidation levels, and several ferroptosis inducers have been developed that specifically target the activity of this enzyme through direct inhibition (e.g., RSL3) (Yang et al. 2014). A second class of ferroptosis inducers (e.g., IKE and erastin) causes inactivation of GPX4 through depletion of glutathione via inhibition of the antiporter system x_{c}⁻ (Larraufie et al. 2015). Ferroptosis has been implicated in several disease pathologies, such as degenerative diseases and organ injury (Jiang et al. 2021; Weiland et al. 2018). Furthermore, ferroptosis induction may have potential as a cancer treatment strategy (Angeli et al. 2019; Chen et al. 2021).

Toward the goal of specific identification of ferroptosis in tissue samples, we previously discovered an effective ferroptosis-staining reagent, 3F3 anti-Ferroptotic Membrane Antibody (3F3-FMA), that can be used to stain cells and tissue samples directly (Feng et al. 2020). The antigenic target of 3F3-FMA is transferrin receptor 1 (TfR1), a membrane receptor that internalizes iron-bound transferrin through receptor-mediated endocytosis (Cheng et al. 2004). This iron uptake activity of TfR1 contributes to intracellular iron levels necessary for ferroptosis (Yang and Stockwell, 2008). 3F3-FMA, as well as other anti-TfR1 antibodies, exhibit an increase in total and membrane-localized fluorescence when used to stain cells undergoing ferroptosis in culture (compared to vehicle-treated control cells). TfR1 has been used to identify the occurrence of ferroptosis in traumatic brain injury (Chen et al. 2021) and myocardial ischemia/reperfusion injury (Fan et al. 2021), among other uses. Thus, TfR1 serves as a biomarker to facilitate the identification of ferroptosis in cell and tissue contexts.

The identification of plasma membrane fluorescence as a distinguishing feature between ferroptosis and other cell death processes upon staining with anti-TfR1 antibodies was discovered using visual inspection; here, we sought instead to evaluate the use of machine learning as an unbiased tool to detect ferroptotic cells. Machine learning methods facilitate the high-throughput analysis of cell image sets versus tedious and subjective manual processes; in cell biology applications, machine learning can increase processing capabilities and objectivity. The supervised machine learning pipeline involves image collection and pre-processing, object detection, and feature extraction and prioritization (Sommer and Gerlich, 2013). Our goals were to assess the machine learning potential in discriminating ferroptosis, apoptosis and control-treated samples as well as to provide a pipeline for identification of features that best distinguish those cell death modalities in our setting.

Therefore, after collecting images of fluorescently-stained cells treated with vehicle only or undergoing ferroptosis or apoptosis, images were analyzed via high-content-image analysis and a classifier was trained on the extracted data. The trained classifier corresponds to a non-exclusive list of informative features with assigned coefficients, which was validated with a second data set by successfully predicting the same classes. These results expand and strengthen the applicability of biomarkers, such as 3F3-FMA/TfR1, for differentiating cell death mechanisms in an objective and high-throughput manner.

### Results and Discussion

To explore the application of machine learning to the classification of different cell death modalities, we collected large numbers of images of cells fixed and immunofluorescently stained with 3F3 anti-Ferroptotic Membrane Antibody (3F3-FMA), a ferroptosis-specific antibody with TfR1 as its target antigen. Specifically, HT-1080 cells were treated with ferroptosis inducers (RSL3, a GPX4 inhibitor, or IKE, a system x_{c}⁻ inhibitor), an apoptosis inducer (staurosporine, STS) (Bertrand et al. 1994), or DMSO vehicle control. In addition to being stained with anti-TfR1 3F3-FMA (labeled with AlexaFluor 594), cells were stained with DAPI as a nuclear marker and FITC-phalloidin as a cytoplasmic (F-actin) marker to assist identification of cellular features for machine learning classification (see below).

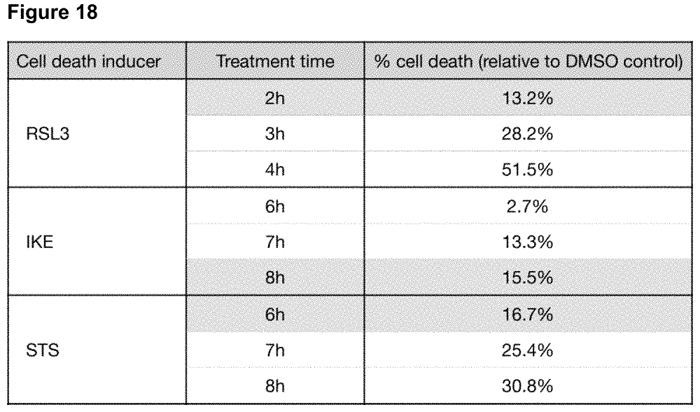

Machine learning tools are designed to adapt to any data pattern associated with the task to learn. There were several important aspects to consider in collecting images for machine learning classification. First, all treatments within a day (i.e., using the same microscope settings) were balanced. Moreover, we collected all images of the discovery data on one day and the validation data later on a different day. Second, the extent of cell death was standardized across the different conditions to analyze cells in an early stage of cell death induction. Specifically, we fixed cells in each treatment condition when they reached 10-20% cell death, so that cell death has been initiated, but not to the extent of excessive end-stage necrosis. At this point, the cells should still have intact cell membrane integrity and not have detached from the surface. The CellTiter-Glo (CTG) viability assay, which measures intracellular ATP levels as an indicator of viability, was used to monitor the extent of cell death. We performed a pilot study and established optimal concentration and timepoint ranges for each treatment (FIG. 18).

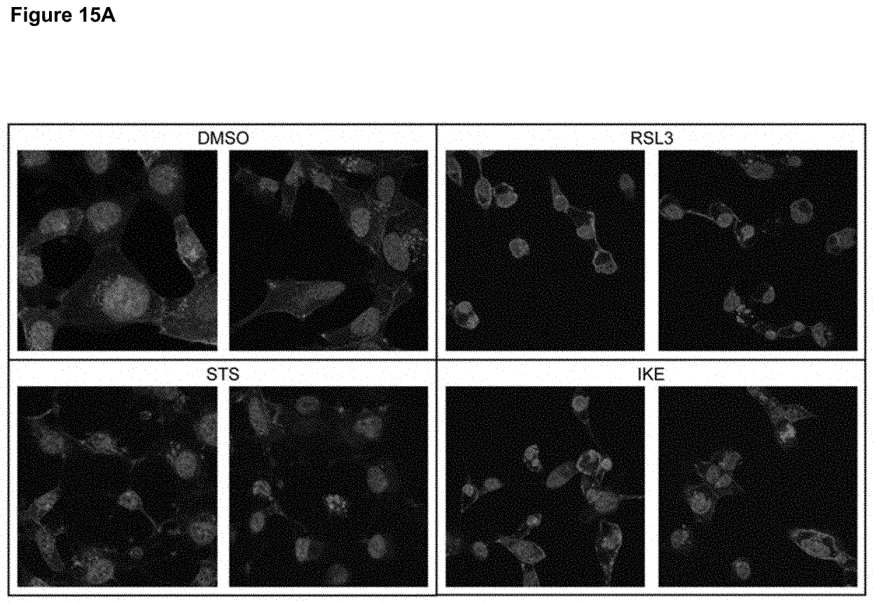

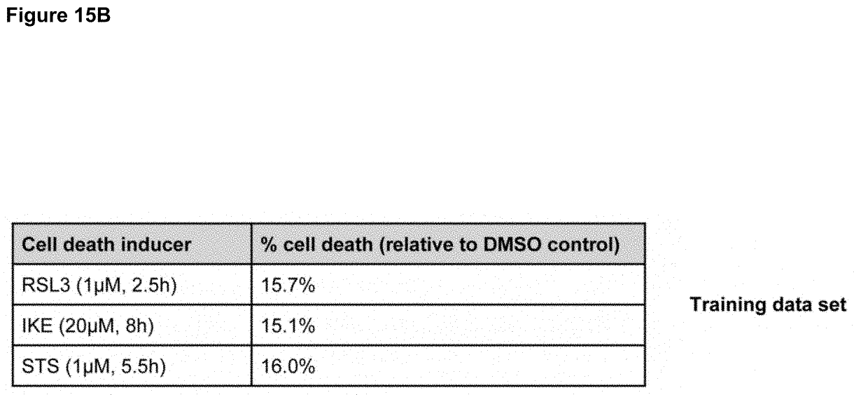

Guided by the results of the pilot study, the first image set for training and discovery of classifiers was collected, and immunofluorescence experiments were performed when the extent of cell death reached 10-20% compared to DMSO control treatment in parallel CTG assays (FIG. 15). Viewing the images, the characteristic membrane localization of 3F3-FMA signal can be seen in ferroptotic cells compared to DMSO control (Feng et al. 2020), and characteristic membrane blebbing can be observed in apoptotic cells (Wyllie et al. 1980).

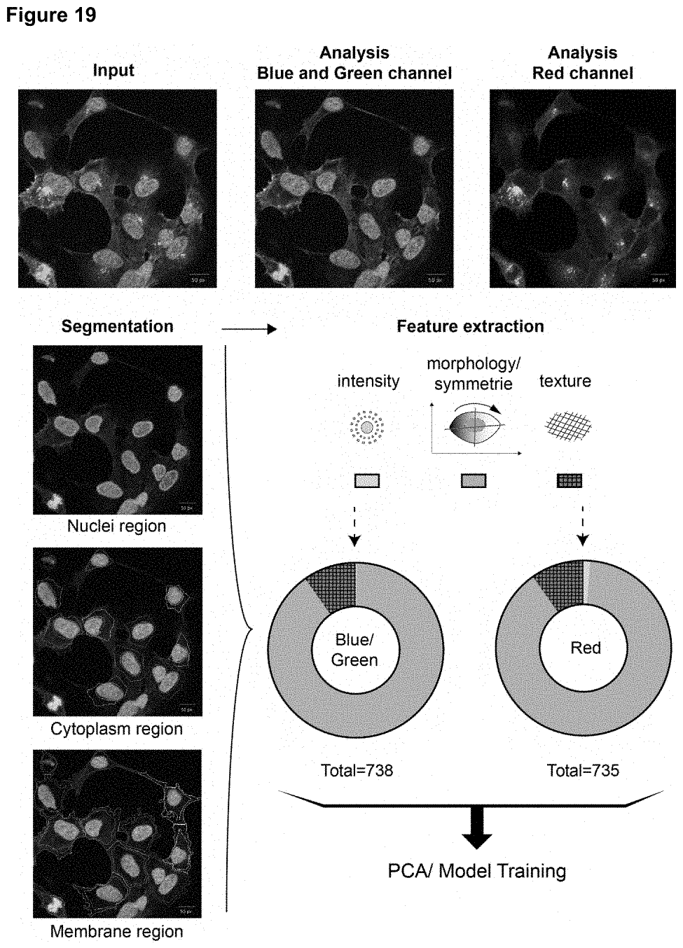

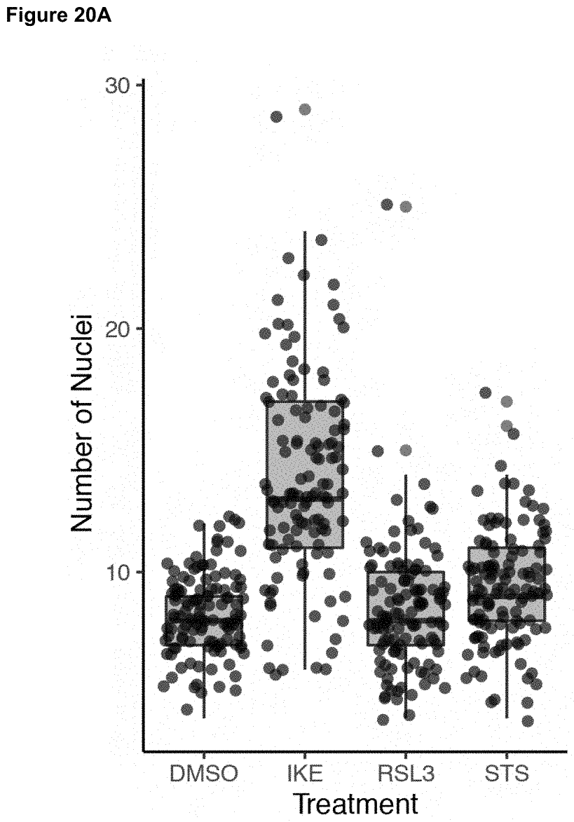

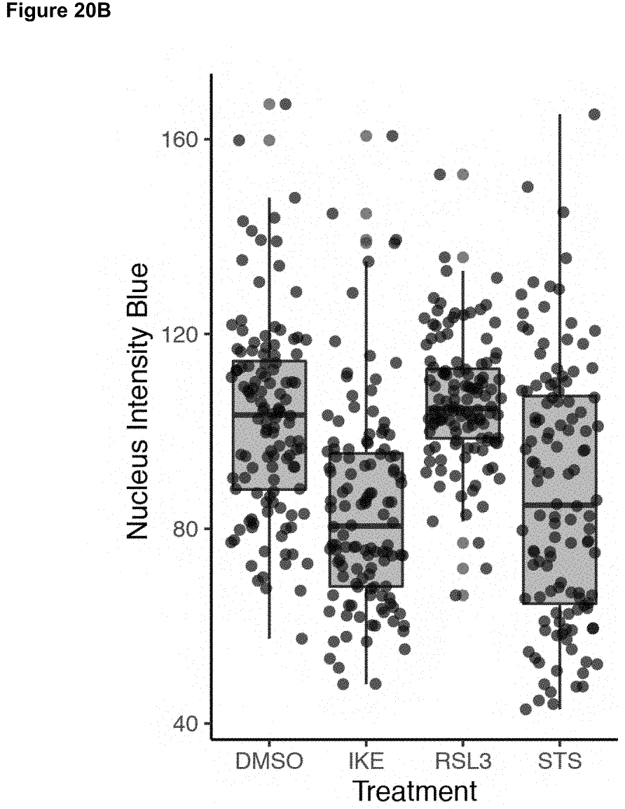

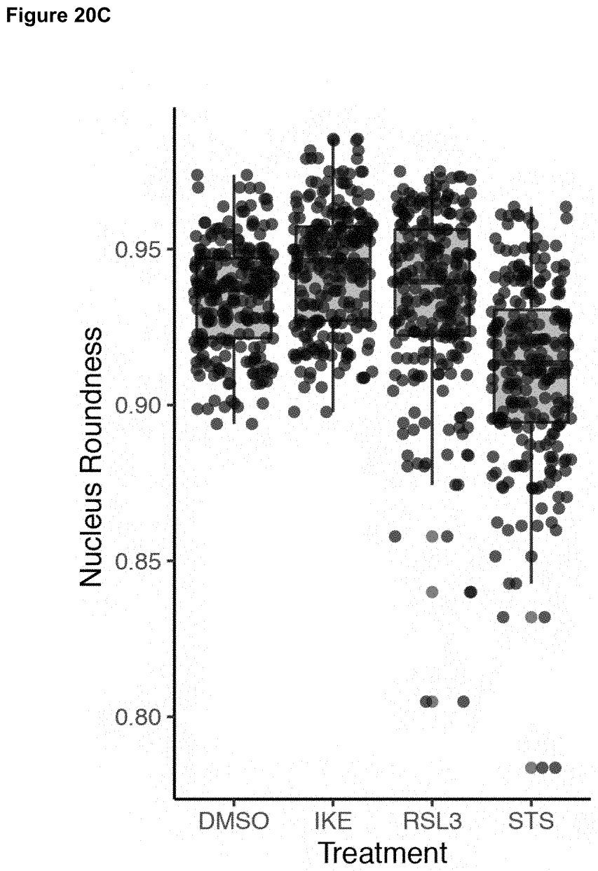

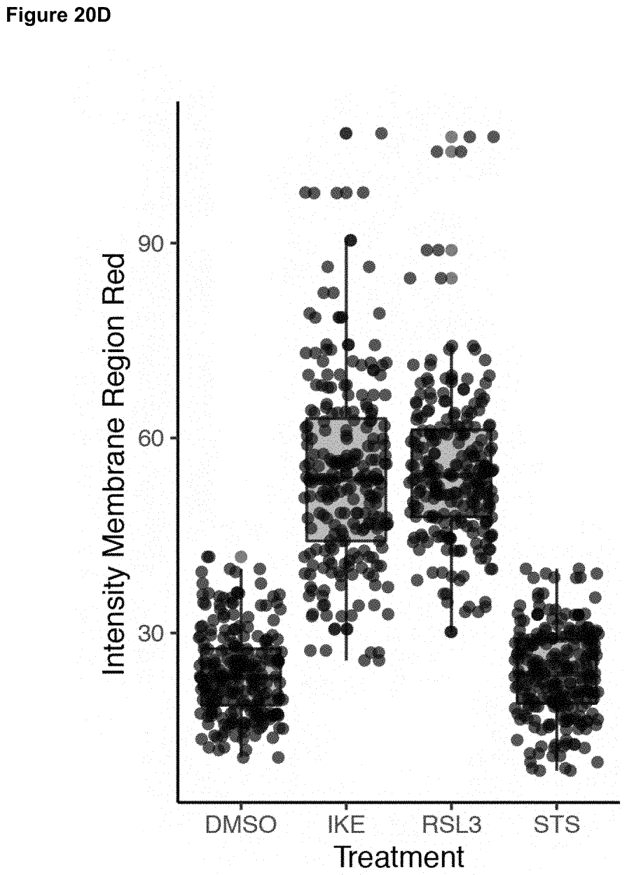

For the training set, once the cells were fixed and stained with DAPI, FITC-phalloidin, and anti-TfR1 3F3-FMA, 120 images were collected per treatment condition (DMSO control, RSL3, IKE, STS) with an average of 10 cells per image (FIG. 2A), which corresponds to a cell density of approximately 80% for DMSO-treated cells. Subsequently, we analyzed images with the PerkinElmer Columbus high-content-analysis software. For this purpose, nuclei were identified using DAPI signal, and based on this, the cytoplasm and the membrane regions were segmented using the F-actin signal (FIG. 19). The intensity, the morphology, and the symmetry of the objects, as well as the texture and structure of the fluorescence signal were determined within these cell segments for the blue, green and red channels, respectively. Consequently, we were able to extract a large number of features for each image. Importantly, during the analysis, the features for single cells were averaged for each image (median). This gave rise to 120 observations per treatment for each feature. The blue (DAPI) and green (FITC-phalloidin) channel provided together 738 features, while the red (TfR1) channel provided 735 features (FIG. 19). Among these features there were frequently used features such as “Number of Nuclei”, “Nucleus Intensity” and “Nucleus Roundness”. As expected, different effects are visible for basic features after treatment, but no reasonable classification could be made (FIGS. 20A-20C). In order to validate the quality of the data, we analyzed the membrane fluorescence intensity for the TfR1 signal. As expected, we found a significant increase in TfR1 fluorescence intensity after treatment with RSL3 and IKE, but not upon treatment with DMSO or STS (FIG. 20D).

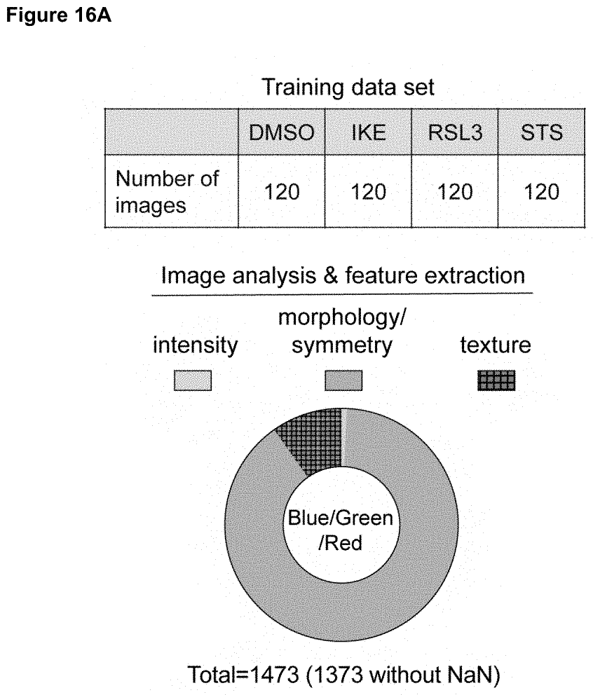

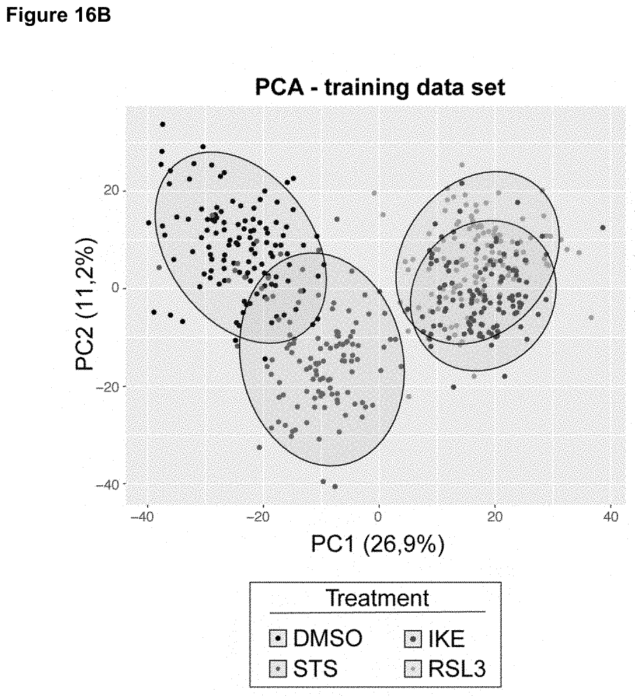

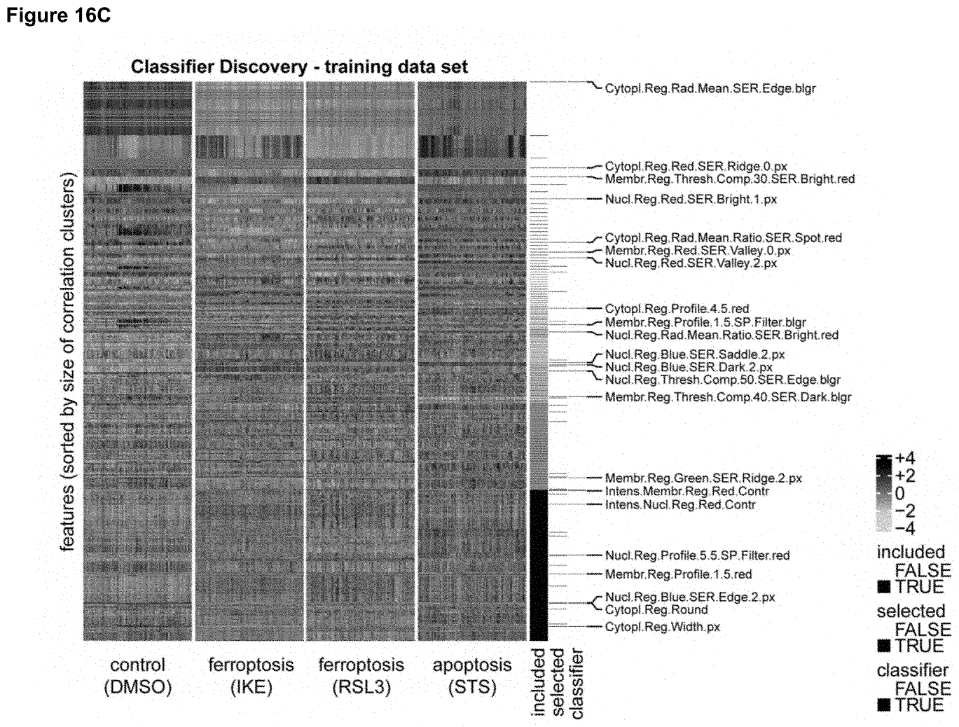

We then removed all features that contained undefined values (NaN, “Not a-Number”) and reduced the number of features from 1,473 to 1,373. We performed a principal component analysis (PCA) with the data matrix of 1,373 features and a total of 480 observations (=120 images per condition; DMSO, IKE, RSL3 and STS) and visualized principal component 1 and 2 (FIG. 16B). The cells treated with RSL3 and IKE separated well from the other samples in the first principal component (FIG. 16B). As expected, the RSL3-treated and IKE-treated samples overlapped in the first two principal components, as both induce the same type of cell death modality, namely ferroptosis. Cells treated with STS also separated from the DMSO population, although to a lesser extent compared to ferroptosis inducers. STS differs not only from the vehicle DMSO, but also from RSL3 and IKE, although cell death in the CTG viability assay performed in parallel was almost identical. This indicated that the staining and analysis strategy was able to distinguish vehicle-treated from ferroptosis, and from apoptosis.

This data set was then used for supervised machine learning to build a classifier that would allow the determination of whether treatments of cells with certain substances trigger ferroptosis or apoptosis (FIG. 16C).

A classifier is a mathematical function or procedure that assigns a sample to one or several classes, usually by calculating class scores for each sample (i.e., image) from its feature values. With respect to the type of mathematical procedure, classifiers vary in terms of interpretability and transferability to new data sets. Multinomial logistic regression models using the lasso (least absolute shrinkage and selection operator) inherently provide a feature selection and return a vector of coefficients for the selected features, called signature, which is directly interpretable and transferable.

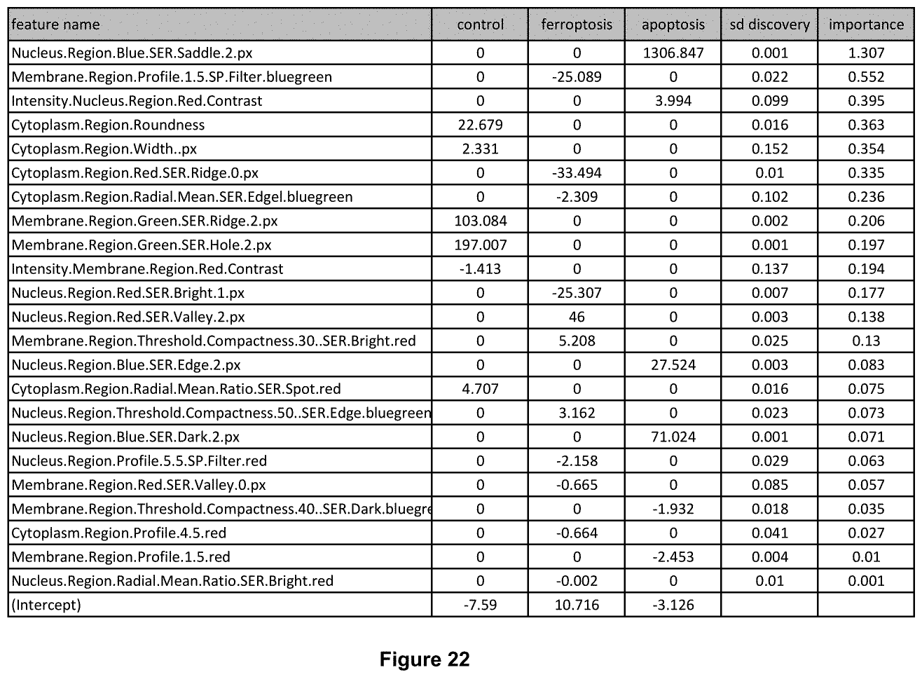

For numerical stability of a treatment classifier, all non-normally distributed features (Shapiro-Wilk test of normality in discovery data, alpha=0.05) were Box-Cox transformed (parameters lambda1=0 and lambda2=1 if the p-value of this test was increased by transformation). Reduction of dimensionality was carried out by removal of redundancies (according to feature-pairwise Pearson correlation of |r|>0.9 in discovery data) and by pre-selection of informative features through treatment-pairwise logistic lasso regression analysis. Notably, only informative features of limited correlation among each other were used for signature discovery. The CRAN package glmnet was used to perform multinomial logistic lasso regression (Friedman et al. 2010). For classification of three groups (DMSO; IKE/RSL3; STS) a signature of 23 features was identified (FIG. 22). These features have biological meanings and can be interpreted as such: for instance, the feature “Membrane.Region.Red.SER.Valley.0.px” is based on texture changes (=SER.Valley.0.px; SER=Spots, Edges and Ridges) of the TfR1 staining (=Red) within the cell membrane (=Membrane.Region). We have previously shown that TfR1 plasma membrane intensity staining changes under ferroptotic conditions (Feng et al. 2020). Thus, it is plausible that this feature should be represented in a classifier signature. Interestingly, the signature also consists of features that are not TfR1 related. For example, the feature “Nucleus. Region. Blue.SER.Saddle.2.px” describes a texture (SER.Saddle.2.px) in the nucleus that is determined using the blue channel (DNA staining). Importantly, this particular texture changes upon treatment with apoptosis inducers, which is expected as apoptosis induces alterations to DNA and chromatin structure. Similar to these two examples, the biological context of features can be interpreted.

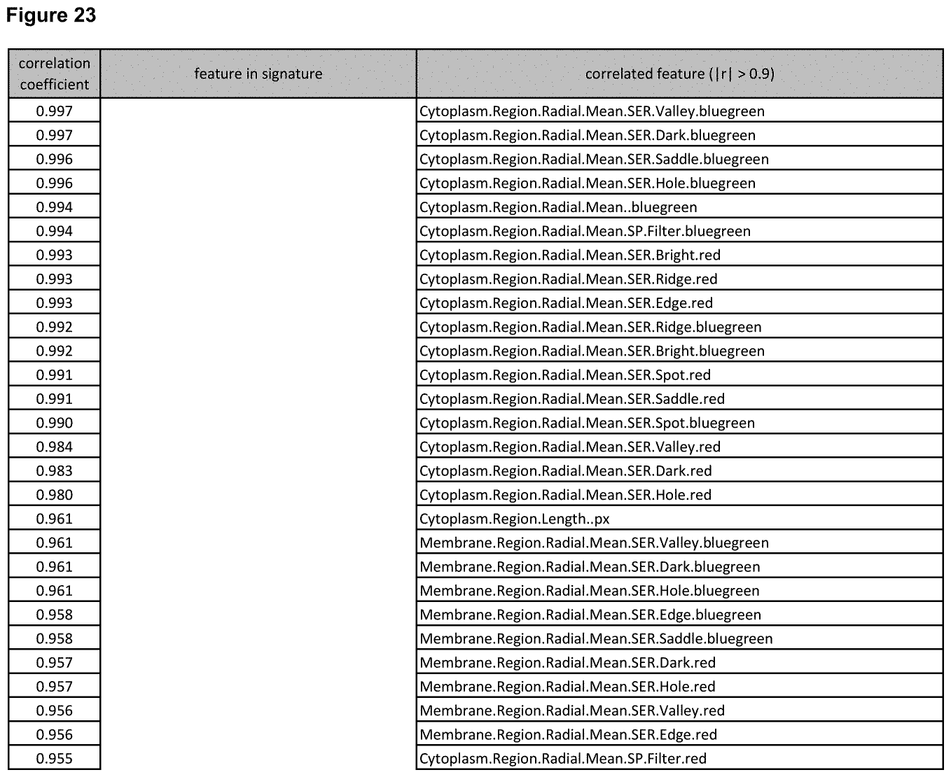

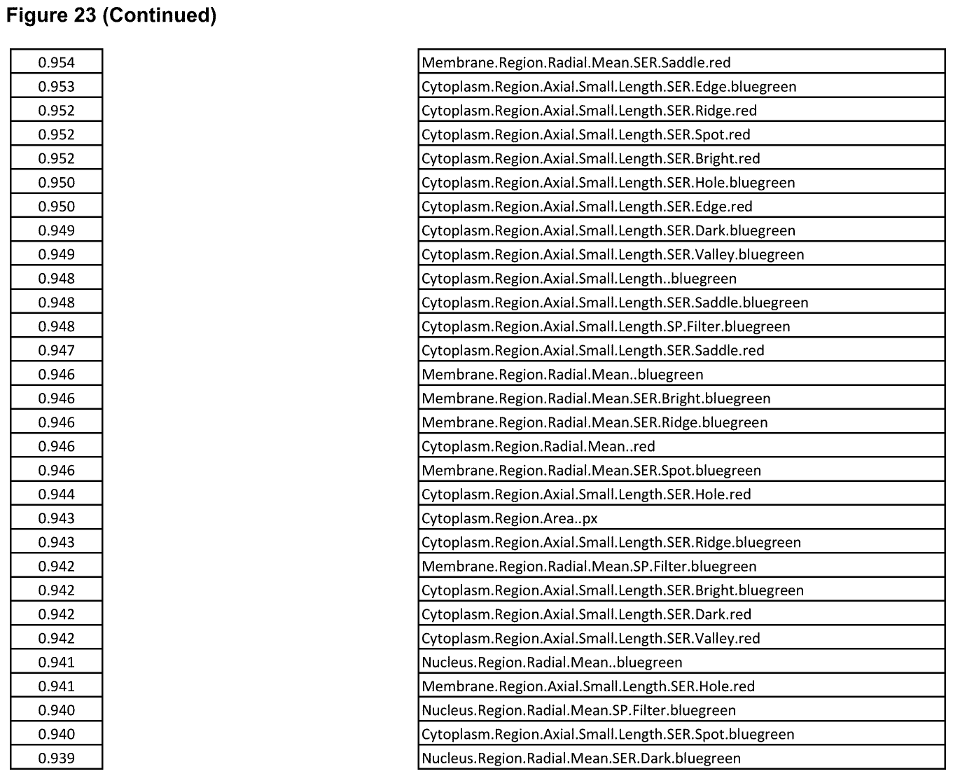

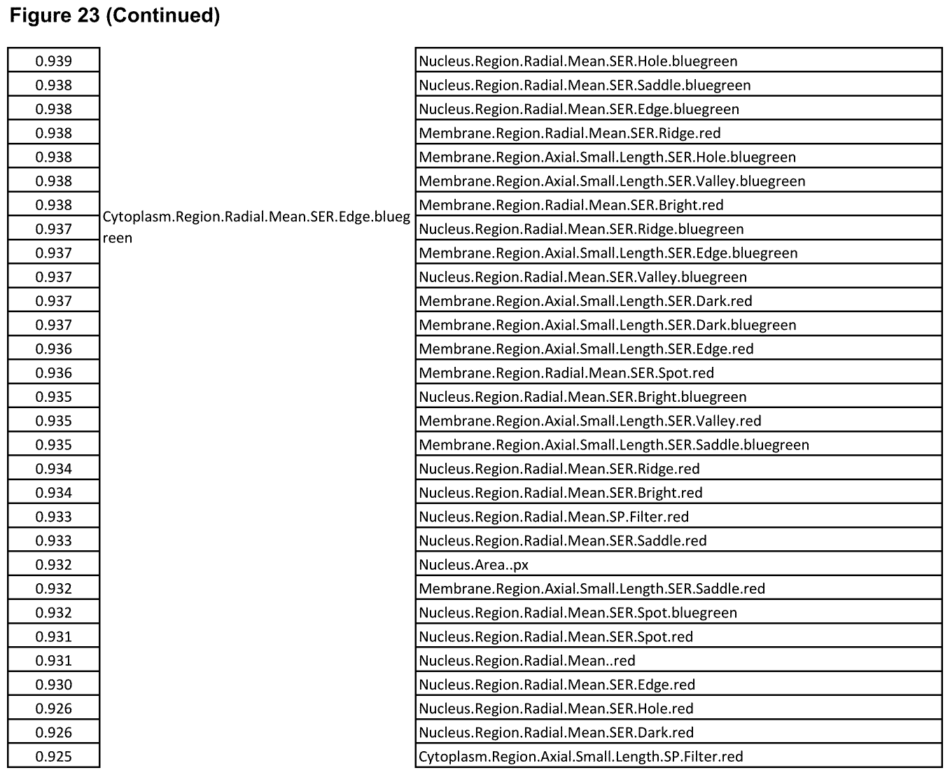

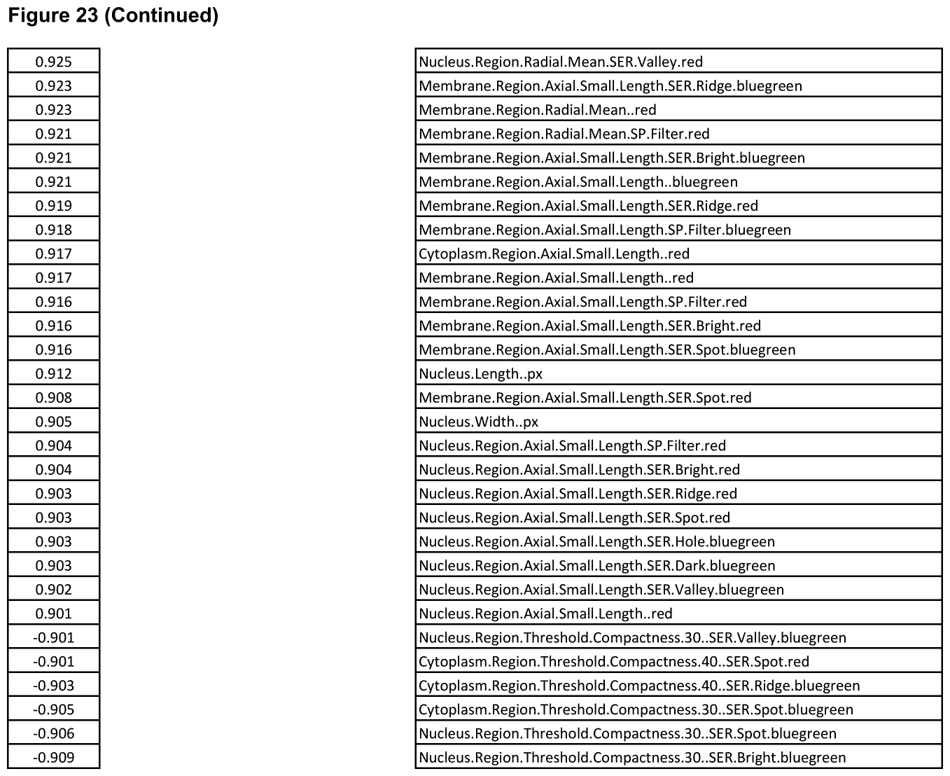

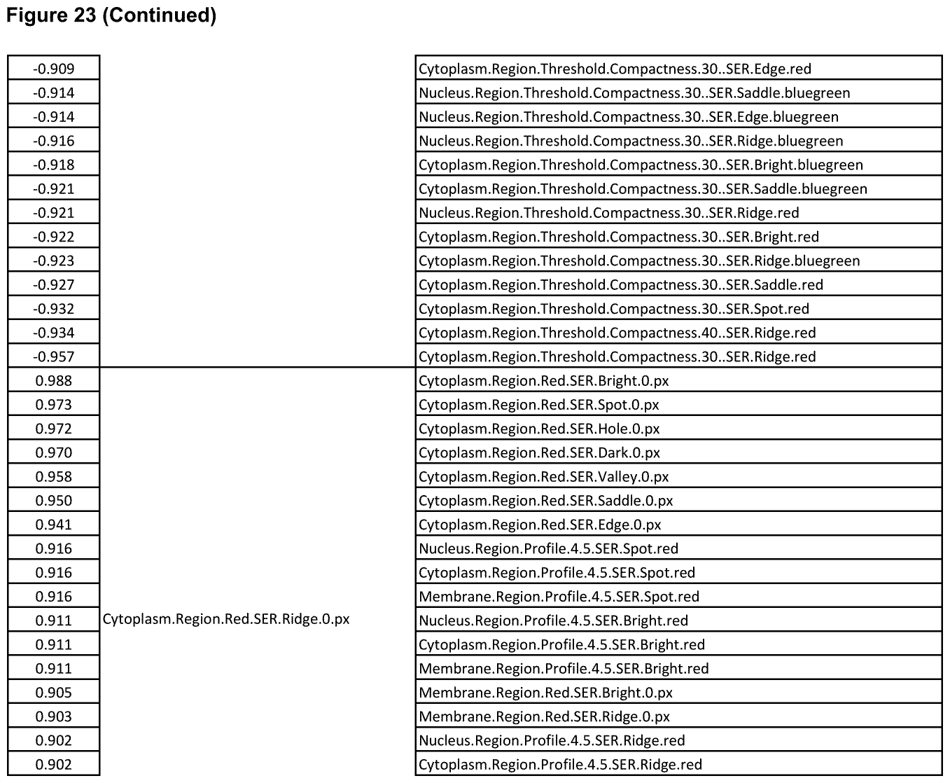

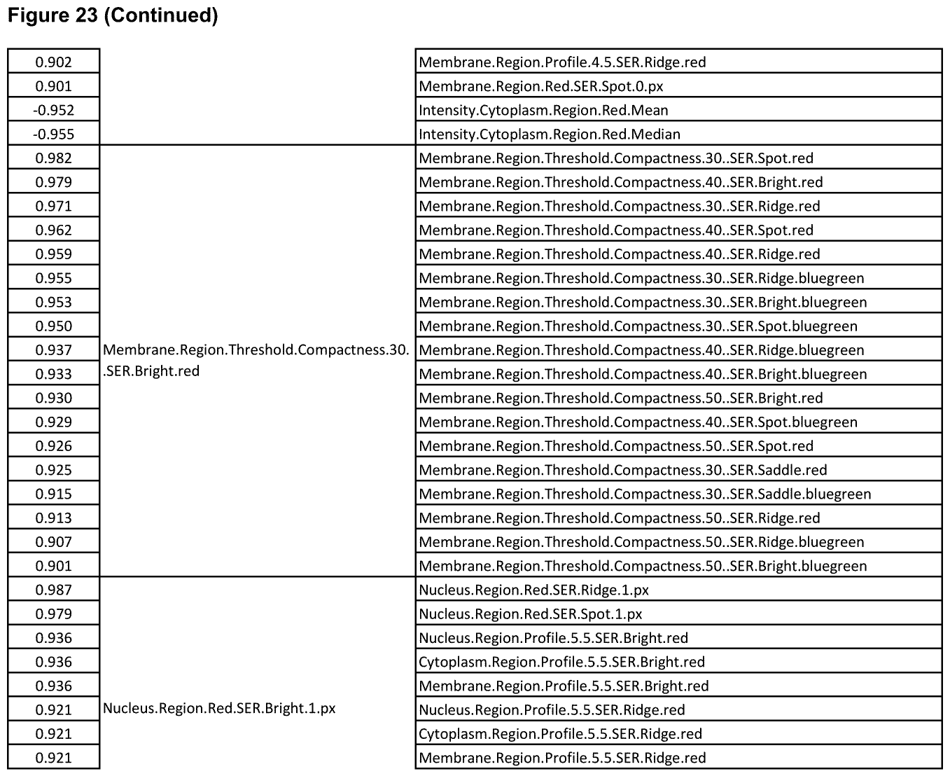

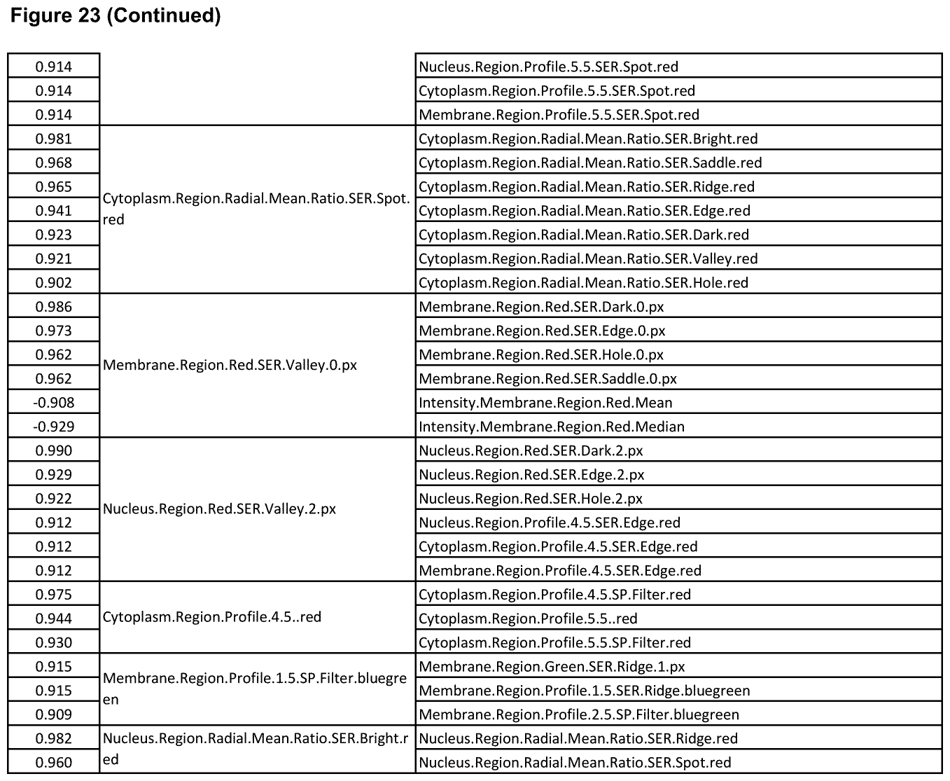

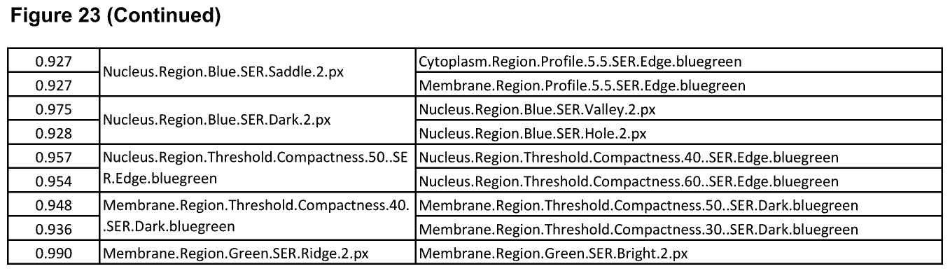

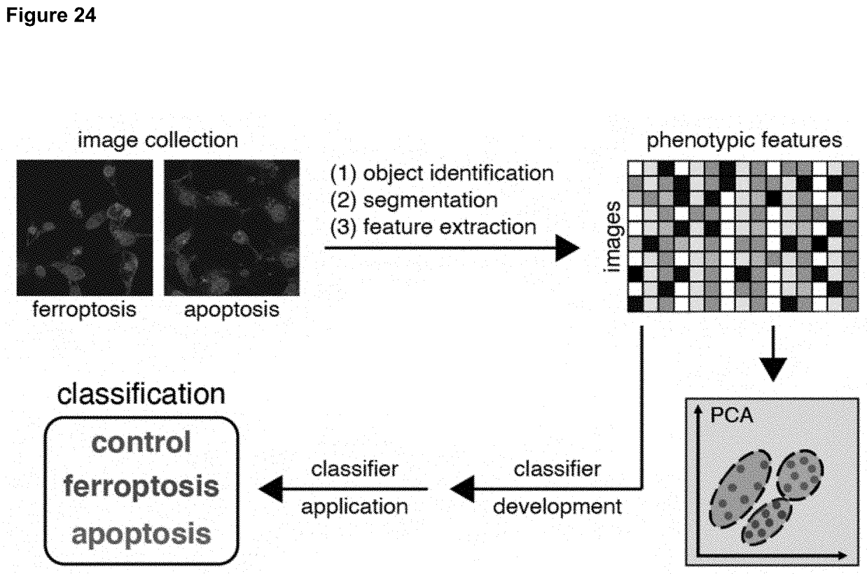

Together, this unbiased approach to classifier identification offers the possibility of discovering features that previously have not been considered in cell death. Hence, this strategy allows the development of a signature using features whose changes human eyes would not necessarily perceive, and helps to more accurately classify cell death states. Notably, there are highly correlated features in the full data set (FIG. 23), which are potentially replaceable in the classifier (after refitting the coefficients). Features that were not included in the classifier are not necessarily uninformative—they were not selected, because they do not contribute additional information to improve the classifier.

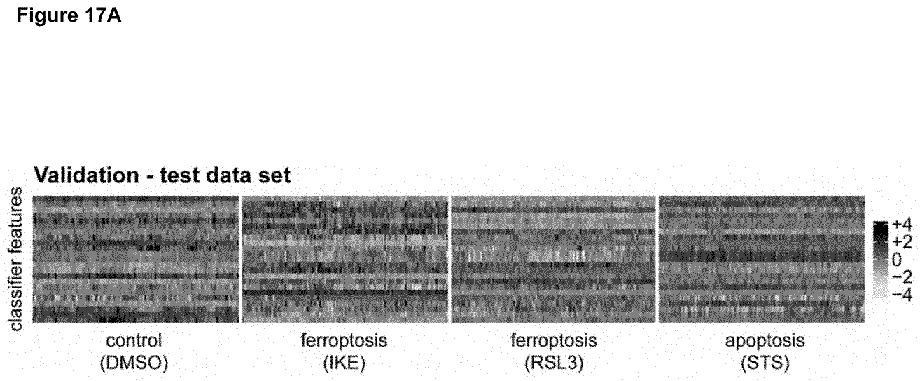

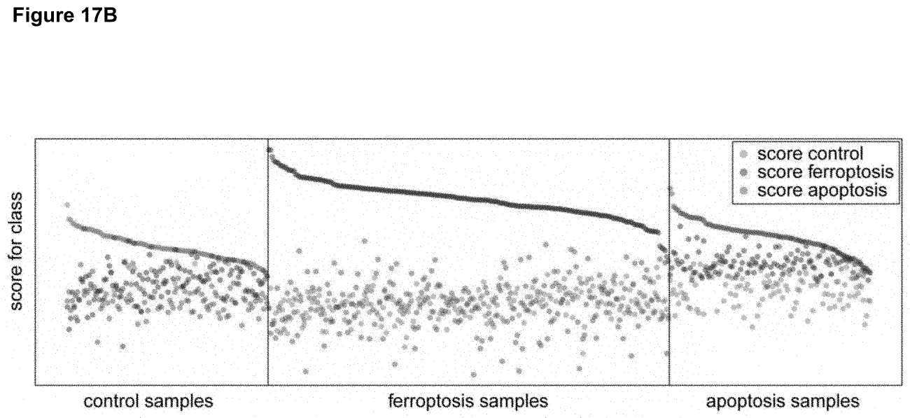

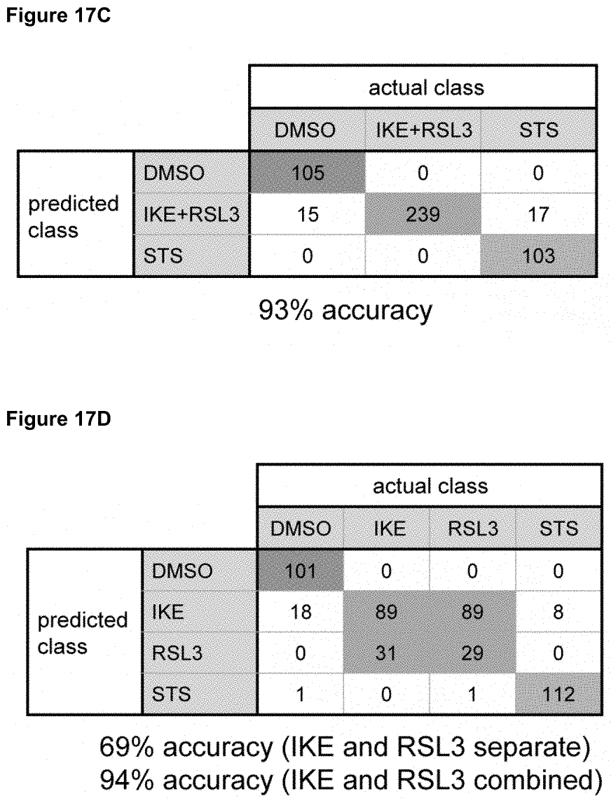

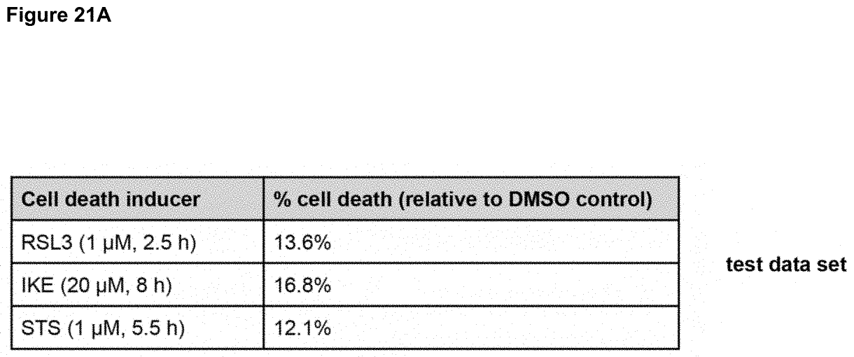

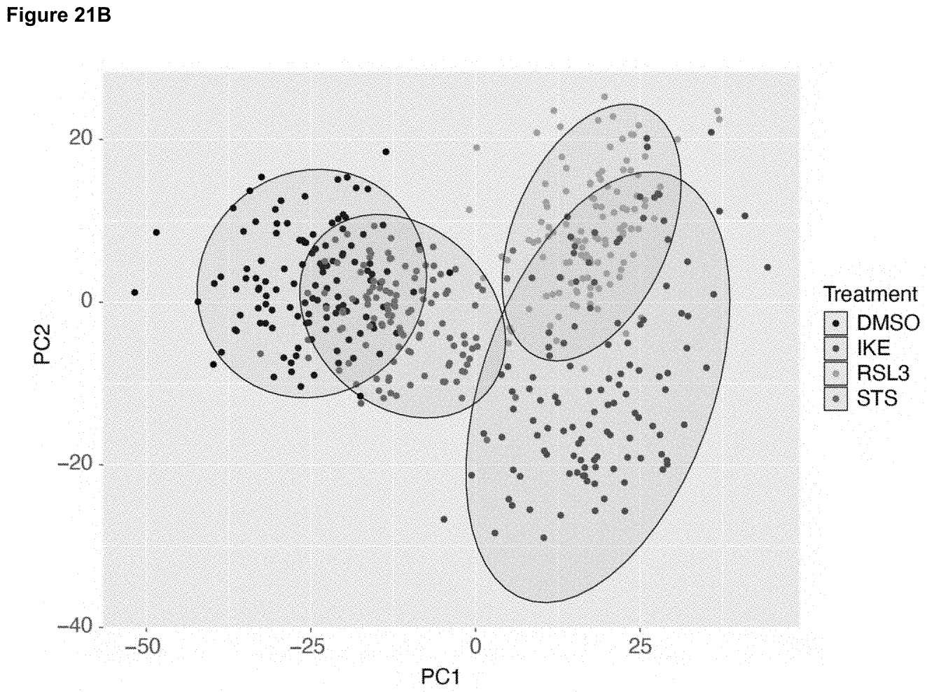

We then collected an independent second image set—using the same conditions with viabilities in the 80-90% range (FIG. 21A)—in order to generate biological replicates for model validation (FIG. 21B). For this experiment, termed the ‘validation experiment’, we ran an identical analysis to extract image data and generated the same set of features as was used in the ‘training experiment’. For model validation, the data from the validation experiment was used to challenge the identified classifier. The coefficients of the 23 features in the classifier were used to predict the class of the samples in the validation experiment, i.e., control, ferroptosis or apoptosis (FIGS. 17A-17B). The accuracy of prediction for the three classes of control (DMSO), ferroptosis (RSL3+IKE) or apoptosis (STS) was 93% (447 out of 479 cases correct) (FIG. 17C).

A four-class classifier trained to distinguish the three inducers (IKE, RSL3 and STS), as well as the DMSO control, did not differentiate between IKE and RSL3, as expected. Both classes were assigned identically to IKE (89 cases each) or RSL3 (31 and 29 cases) and minimally to STS (0 or 1 case). Combining IKE and RSL3 resulted in an accuracy of 94% (FIG. 17D). Consistently, even when excluded from model discovery, IKE validation set images were constantly identified as RSL3-like by two-class logistic lasso regression classifiers trained to discriminate DMSO control from RSL3 or STS from RSL3 (120 of 120 and 113 of 120 images, respectively—see supplementary PDF file “MachineLearning_Ferroptosis_SI.pdf”: “Binary Prediction”). Importantly, this suggests that both ferroptosis inducers induce a similar phenomenology with respect to the features extracted from the images.

The classifier performed well for detecting ferroptosis, as TfR1 is a known ferroptosis marker, and features from this channel are prominently represented in the signature. However, we were intrigued that apoptosis was also readily distinguished from the control group using the developed signature.

This classifier is based on images of cells treated with ferroptosis or apoptosis inducers and stained with anti-TfR1 3F3-FMA, DAPI and FITC-Phalloidin. It is important to consider that for any new (unknown) small molecule that is desired to be tested with this classifier, the concentration and incubation times reducing the viability to 80-90% have to be identified in advance. Standardized microscopy image acquisition of treated cells in combination with this classifier could provide the information of whether the substances induce ferroptosis or apoptosis. As with any analysis tool, some refinement might be needed.

Further, this work may have important implications for tissue analysis and allow for a high-throughput, objective procedure to identify ferroptosis and other cell death modalities in a tissue context, whether with animal disease models or patient samples. One such application may involve assessing the response of cancer patients to therapy (Chen et al. 2021).

This classifier cannot directly be applied to images taken under entirely different conditions (treatments, staining, etc.). However, we present a workflow on how researchers can develop a classifier based on a training image set for various cell death processes with the help of standardization of experiments and corresponding analysis tools. Hence, this strategy may serve as a blueprint to be employed for the detection of other cell death pathways, including necroptosis and pyroptosis, and ultimately a universal classifier that detects and classifies all of the major types of cell death.

### Methods

Cell culture. HT-1080 (ATCC Cat #CRL-7951, RRID:CVCL 0317) cells were grown in Dulbecco's Modified Eagle Medium (DMEM) with 10% fetal bovine serum, 1% penicillin-streptomycin, and 1% non-essential amino acids. Cells were grown in a humidified incubator at 37° C. and 5% CO₂.

CellTiter-Glo assay. HT-1080 cells were plated in technical triplicates in white opaque 96-well plates at 15,000 cells/100 μL media per well. For the pilot experiment, the cells were treated with 1 μM RSL3, 20 μM IKE, or 1 μM staurosporine (STS) at different time points. For the immunofluorescence experiments, the cells were treated at the time points determined in the pilot experiment and several time points before and after. 100 μL of 50% CellTiter-Glo (Promega) and 50% cell culture medium was added to each well, and the cells were incubated and shaken for 2 min at room temperature. Luminescence was measured using a Victor X5 plate reader (PerkinElmer).

Immunofluorescence (IF). HT-1080 cells were treated with 1 μM RSL3, 20 μM IKE, or 1 μM STS on poly-lysine-coated coverslips (Sigma Aldrich P4832) in 24-well plates. When the cell death percentage reached around 10-20% (determined using the CellTiter-Glo assay), media was removed and the cells were gently washed with PBS⁺⁺ (PBS with 1 mM CaCl₂) and 0.5 mM MgCl₂) twice, ensuring the cells did not dry out. The cells were fixed and permeabilized with 4% PFA in PBS with 0.1% Triton X-100 (PBT), with 200 μL per well. The plates were covered with foil, and the cells were incubated and shaken at room temperature for 15-20 min. The PFA was disposed of safely, and the cells were washed with PBT three times. The cells were blocked with 5% normal goat serum (NGS) (ThermoFisher 50197Z) in PBT for 1 h at room temperature. The cells were then incubated with mouse 3F3 anti-Ferroptotic Membrane Antibody (3F3-FMA) at 1:500 dilution in PBT with 1% bovine serum albumin (BSA) and 5% NGS at 4° C. overnight. The cells were washed with PBT for 5 min three times. The cells were then incubated with goat anti-mouse IgG (H+L) Highly Cross-Adsorbed Secondary Antibody, Alexa Fluor 594 (Thermo Fisher Scientific Cat #A-11032, RRID:AB_2534091) at 1:200 dilution and FITC-phalloidin at 1:1000 dilution in PBT with 1% BSA for 1 h at room temperature. The cells were washed with PBT for 5 min three times. The cells were placed on slides using Prolong Diamond anti-fade mountant with DAPI (ThermoFisher P36962). All images were collected on a Zeiss LSM 800 confocal microscope at Plan-Apochromat 63×/1.40 oil DIC objective with constant laser intensity for all images.

Automated image analysis. Image analysis was performed using Columbus software version 2.8.0 (PerkinElmer). In the following, the analysis steps in Columbus are described: DAPI and FITC signal were smoothened for the cell segmentation process using Median filters to reduce noise signals. Nuclei were detected via the DAPI signal. The FITC channel was used to define the cytoplasm and membrane region. In a next step, morphology/symmetry features, texture (SER features) and intensity properties of the DAPI, FITC and red channel were calculated for each cell region (nuclei, cytoplasm, and membrane). Moreover, we applied a filter to remove border objects (nuclei that cross image borders). For the detailed analysis pipeline in Columbus please see FIG. 19 and the following analysis sequences.

ANALYSIS SEQUENCES

| Blue-Green: | | | |
|---|---|---|---|
| Input | Stack Processing: | | |
| Image | Individual Planes | | |
| | Flatfield Correction: | | |
| | None | | |
| Filter | Channel: Blue | Method: Sliding Parabola | Output Image: |
| Image | | Curvature: 10 | Sliding |
| | | | Parabola |
| Filter | Channel: Sliding Parabola | Method: Smoothing | Output Image: |
| Image (2) | | Filter: Median | Median |
| | | Scale: 6 px | Smoothed Nuclei |
| Filter | Channel: Green | Method: Smoothing | Output Image: |
| Image | | Filter: Median | Median |
| (3) | | Scale: 3 px | Smoothed |
| Find | Channel: Median | Method: B | Output Population: |
| Nuclei | Smoothed | Common Threshold: 0.4 | Nuclei |
| | Nuclei | Area: >150 px2 | |
| | ROI: None | Split Factor: 10 | |
| | | Individual Threshold: 0.4 | |
| | | Contrast: >0.1 | |
| Calculate | Channel: Blue | Method: Standard | Output Properties: |
| Intensity | Population: Nuclei | Mean | Intensity |
| Properties | Region: Nucleus | | Nucleus Blue |
| Calculate | Population: Nuclei | Method: Standard | Output Properties: |
| Morphology | Region: Nucleus | Area | Nucleus |
| Properties | | Roundness | |
| | | Width | |
| | | Length | |
| | | Ratio Width to Length | |
| | | Output Properties: | |
| | | Nucleus | |
| Select | Population: Nuclei | Method: Filter by Property | Output Population: |
| Population | | Intensity Nucleus Blue Mean: | Nuclei |
| | | >35 | Selected |
| | | Nucleus Roundness: >0.6 | |
| | | Nucleus Area [px2]: >300 | |
| | | Boolean Operations: F1 and | |
| | | F2 and F3 | |
| Find | Channel: Median | Method: A | Output Population: |
| Cytoplasm | Smoothed | Individual Threshold: 0.15 | Nuclei |
| | Nuclei: Nuclei Selected | | |
| Select | Population: Nuclei | Method: Common Filters | Output Population: |
| Population | Selected | Remove Border Objects | Nuclei Selected |
| (2): | | Region: Nucleus | Selected |
| Select | Population: Nuclei | Method: Resize Region [%] | Output Region: |
| Cell | Selected | Region Type: Cytoplasm | Cytoplasm |
| Region | Selected | Region | Region |
| | | Outer Border: 15% | |
| | | Inner Border: 40% | |
| Select | Population: Nuclei | Method: Resize Region [%] | Output Region: |
| Cell | Selected | Region Type: Membrane | Membrane |
| Region | Selected | Region | Region |
| (2) | | Outer Border: −10% | |
| | | Inner Border: 10% | |
| Select | Population: Nuclei | Method: Resize Region [%] | Output Region: |
| Cell | Selected | Region Type: Nucleus | Nucleus |
| Region | Selected | Region | Region |
| (3) | | Outer Border: 55% | |
| | | Inner Border: 100% | |
| Calculate | Channel: Blue | Method: SER Features | Output Properties: |
| Texture | Population: Nuclei | Scale: 0 px | Nucleus |
| Properties | Selected | Normalization by: Kernel | Region Blue |
| (1) | Selected | SER Spot | |
| | Region: Nucleus Region | SER Hole | |
| | | SER Edge | |
| | | SER Ridge | |
| | | SER Valley | |
| | | SER Saddle | |
| | | SER Bright | |
| | | SER Dark | |
| Calculate | Channel: Blue | Method: SER Features | Output Properties: |
| Texture | Population: Nuclei | Scale: 1 px | Nucleus |
| Properties | Selected | Normalization by: Kernel | Region Blue |
| (2) | Selected | SER Spot | |
| | | SER Hole | |
| | | SER Edge | |
| | | SER Ridge | |
| | | SER Valley | |
| | | SER Saddle | |
| | | SER Bright | |
| | | SER Dark | |
| Calculate | Channel: Blue | Method: SER Features | Output Properties: |
| Texture | Population: Nuclei | Scale: 2 px | Nucleus |
| Properties | Selected | Normalization by: Kernel | Region Blue |
| (3) | Selected | SER Spot | |
| | Region: Nucleus Region | SER Hole | |
| | | SER Edge | |
| | | SER Ridge | |
| | | SER Valley | |
| | | SER Saddle | |
| | | SER Bright | |
| | | SER Dark | |
| Calculate | Population: Nuclei | Method: STAR | Output Properties: |
| Morphology | Selected | Channel: Blue | Nucleus Region |
| Properties | Selected | Symmetry | |
| (2) | Region: Nucleus Region | Threshold Compactness | |
| | | Axial | |
| | | Radial | |
| | | Profile | |
| | | Profile Inner Region: Nucleus | |
| | | Profile Width: 10 px | |
| | | Sliding Parabola | |
| | | Sliding Parabola | |
| | | Curvature: 10 | |
| | | Use for Center | |
| | | Texture SER | |
| | | Scale: 1 px | |
| | | Normalization by: Kernel | |
| | | SER Spot | |
| | | SER Hole | |
| | | SER Edge | |
| | | SER Ridge | |
| | | SER Valley | |
| | | SER Saddle | |
| | | SER Bright | |
| | | SER Dark | |
| Calculate | Channel: Green | Method: Standard | Output Properties: |
| Intensity | Population: Nuclei | Mean | Intensity |
| Properties | Selected | | Cytoplasm Region |
| (2) | Selected | | Green |
| | Region: Cytoplasm | | |
| | Region | | |
| Calculate | Population: Nuclei | Method: Standard | Output Properties: |
| Morphology | Selected | Area | Cytoplasm Region |
| Properties | Selected | Roundness | |
| (3) | Region: Cytoplasm | Width | |
| | Region | Length | |
| | | Ratio Width to Length | |
| Calculate | Channel: Green | Method: SER Features | Output Properties: |
| Texture | Population: Nuclei | Scale: 0 px | Cytoplasm Region |
| Properties | Selected | Normalization by: Kernel | Green |
| (4) | Selected | SER Spot | |
| | Region: Cytoplasm | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Green | Method: SER Features | Output Properties: |
| Texture | Population: Nuclei | Scale: 1 px | Cytoplasm Region |
| Properties | Selected | Normalization by: Kernel | Green |
| (5) | Selected | SER Spot | |
| | Region: Cytoplasm | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Green | Method: SER Features | Output Properties: |
| Texture | Population: Nuclei | Scale: 2 px | Cytoplasm Region |
| Properties | Selected | Normalization by: Kernel | Green |
| (6) | Selected | SER Spot | |
| | Region: Cytoplasm | SER Hole | |
| | Region | SER Edge | |
| | | SER Ridge | |
| | | SER Valley | |
| | | SER Saddle | |
| | | SER Bright | |
| | | SER Dark | |
| Calculate | Population: Nuclei | Method: STAR | Output Properties: |
| Morphology | Selected | Channel: Green | Cytoplasm Region |
| Properties | Selected | Symmetry | |
| (4) | Region: Cytoplasm | Threshold Compactness | |
| | Region | Axial | |
| | | Radial | |
| | | Profile | |
| | | Profile Inner Region: Nucleus | |
| | | Profile Width: 10 px | |
| | | Sliding Parabola | |
| | | Sliding Parabola | |
| | | Curvature: 10 | |
| | | Use for Center | |
| | | Texture SER | |
| | | Scale: 1 px | |
| | | Normalization by: Kernel | |
| | | SER Spot | |
| | | SER Hole | |
| | | SER Edge | |
| | | SER Ridge | |
| | | SER Valley | |
| | | SER Saddle | |
| | | SER Bright | |
| | | SER Dark | |
| Calculate | Channel: Green | Method: SER Features | Output Properties: |
| Texture | Population: Nuclei | Scale: 0 px | Membrane Region |
| Properties | Selected | Normalization by: Kernel | Green |
| (7) | Selected | SER Spot | |
| | Region: Membrane | SER Hole | |
| | Region | SER Edge | |
| | | SER Ridge | |
| | | SER Valley | |
| | | SER Saddle | |
| | | SER Bright | |
| | | SER Hole | |
| | | SER Edge | |
| | | SER Ridge | |
| | | SER Valley | |
| | | SER Saddle | |
| | | SER Bright | |
| | | SER Dark | |
| Calculate | Channel: Green | Method: SER Features | Output Properties: |
| Texture | Population: Nuclei | Scale: 1 px | Membrane Region |
| Properties | Selected | Normalization by: Kernel | Green |
| (8) | Selected | SER Spot | |
| | Region: Membrane | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Green | Method: SER Features | Output Properties: |
| Texture | Population: Nuclei | Scale: 2 px | Membrane Region |
| Properties | Selected | Normalization by: Kernel | Green |
| (9) | Selected | SER Spot | |
| | Region: Membrane | SER Hole | |
| | Region | SER Edge | |
| | | SER Ridge | |
| | | SER Valley | |
| | | SER Saddle | |
| | | SER Bright | |
| | | SER Dark | |
| Calculate | Population: Nuclei | Method: STAR | Output Properties: |
| Morphology | Selected | Channel: Green | Membrane Region |
| Properties | Selected | Symmetry | |
| (5) | Region: Membrane | Threshold Compactness | |
| | Region | Axial | |
| | | Radial | |
| | | Profile | |
| | | Profile Inner Region: Nucleus | |
| | | Profile Width: 10 px | |
| | | Sliding Parabola | |
| | | Sliding Parabola | |
| | | Curvature: 10 | |
| | | Use for Center | |
| | | Texture SER | |
| | | Scale: 1 px | |
| | | Normalization by: Kernel | |
| | | SER Spot | |
| | | SER Hole | |
| | | SER Edge | |
| | | SER Ridge | |
| | | SER Valley | |
| | | SER Saddle | |
| | | SER Bright | |
| | | SER Dark | |
| Red: | | | |
| Input | Stack Processing: | | |
| Image | Individual Planes | | |
| | Flatfield Correction: | | |
| | None | | |
| Filter | | | |
| Image | | Curvature: 10 | Parabola |
| Filter | Channel: Sliding Parabola | Method: Smoothing | Output Image: Median |
| Image | | Filter: Median | Smoothed Nuclei |
| (2) | | Scale: 6 px | |
| Filter | Channel: Green | Method: Smoothing | Output Image: Median |
| Image | | Filter: Median | Smoothed |
| (3) | | Scale: 3 px | |
| Find | Channel: Median | Method: B | Output Population: |
| Nuclei | Smoothed | Common Threshold: 0.4 | Nuclei |
| | Nuclei | Area: >150 px2 | |
| | ROI: None | Split Factor: 10 | |
| | | Individual Threshold: 0.4 | |
| | | Contrast: >0.1 | |
| Calculate | Channel: Blue | Method: Standard | Output Properties: |
| Intensity | Population: Nuclei | Mean | Intensity |
| Properties | Region: Nucleus | | Nucleus Blue |
| Calculate | Population: Nuclei | Method: Standard | Output Properties: |
| Morphology | Region: Nucleus | Area | Nucleus |
| Properties | | Roundness | |
| | | Width | |
| | | Length | |
| | | Ratio Width to Length | |
| Select | Population: Nuclei | Method: Filter by Property | Output Population: |
| Population | | Intensity Nucleus Blue Mean | Nuclei |
| | | >35 | Selected |
| | | Nucleus Roundness: >0.6 | |
| | | Nucleus Area [px2]: >300 | |
| | | Boolean Operations: F1 and | |
| | | F2 and F3 | |
| Find | Channel: Median | Method: A | |
| Cytoplasm | Smoothed | Individual Threshold: 0.15 | |
| | Nuclei: Nuclei Selected | | |
| Select | Population: Nuclei | Method: Common Filters | Output Population: |
| Population | Selected | Remove Border Objects | Nuclei |
| (2) | | Region: Nucleus | Selected Selected |
| Select | Population: Nuclei | Method: Resize Region [%] | Output Region: |
| Cell | Selected | Region Type: Membrane | Membrane |
| Region | Selected | Region | Region |
| | | Outer Border: −10% | |
| | | Inner Border: 10% | |
| Select | Population: Nuclei | Method: Resize Region [%] | Output Region: |
| Cell | Selected | Region Type: Cytoplasm | Cytoplasm |
| Region | Selected | Region | Region |
| (2) | | Outer Border: 15% | |
| | | Inner Border: 40% | |
| Select | Population: Nuclei | Method: Resize Region [%] | Output Region: |
| Cell | Selected | Region Type: Nucleus | Nucleus |
| Region | Selected | Region | Region |
| (3) | | Outer Border: 55% | |
| | | Inner Border: 100% | |
| Calculate | Channel: Red | Method: Standard | Output Properties: |
| Intensity | Population: Nuclei | Mean | Intensity |
| Properties | Selected | Median | Membrane Region |
| (2) | Selected | Contrast | Red |
| | Region: Membrane | | |
| | Region | | |
| Calculate | Channel: Red | Method: Standard | Output Properties: |
| Intensity | Population: Nuclei | Mean | Intensity |
| Properties | Selected | Median | Cytoplasm Region |
| (3) | Selected | Contrast | Red |
| | Region: Cytoplasm | | |
| | Region | | |
| Calculate | Channel: Red | Method: Standard | Output Properties: |
| Intensity | Population: Nuclei | Mean | Intensity |
| Properties | Selected | Median | Nucleus Region Red |
| (4) | Selected | Contrast | |
| | Region: Nucleus Region | | |
| Calculate | Channel: Red | Method: SER Features | Output Properties: |
| Texture | Population: Nuclei | Scale: 0 px | Membrane Region |
| Properties | Selected | Normalization by: Kernel | Red |
| | Selected | SER Spot | |
| | Region: Membrane | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 0 px | Cytoplasm Region |
| Properties | Selected | Normalization by: Kernel | Red |
| (2) | Selected | SER Spot | |
| | Region: Cytoplasm | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 0 px | Nucleus |
| Properties | Selected | Normalization by: Kernel | Region Red |
| (3) | Selected | SER Spot | |
| | Region: Nucleus Region | SER Hole | |
| | | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 1 px | Membrane Region |
| Properties | Selected | Normalization by: Kernel | Red |
| (4) | Selected | SER Spot | |
| | Region: Membrane | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 1 px | Cytoplasm Region |
| Properties | Selected | Normalization by: Kernel | Red |
| (5) | Selected | SER Spot | |
| | Region: Cytoplasm | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 1 px | Nucleus |
| Properties | Selected | Normalization by: Kernel | Region Red |
| (6) | Selected | SER Spot | |
| | Region: Nucleus Region | SER Hole | |
| | | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 2 px | Membrane Region |
| Properties | Selected | Normalization by: Kernel | Red |
| (7) | Selected | SER Spot | |
| | Region: Membrane | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 2 px | Cytoplasm Region |
| Properties | Selected | Normalization by: Kernel | Red |
| (8) | Selected | SER Spot | |
| | Region: Cytoplasm | SER Hole | |
| | Region | SER Edge | |
| | | SER Ridge | |
| | | SER Valley | |
| | | SER Saddle | |
| | | SER Bright | |
| | | SER Dark | |
| Calculate | Channel: Red | Method: SER Features | Output Properties: |
| Texture | Population: Nuclei | Scale: 2 px | Nucleus |
| Properties | Selected | Normalization by: Kernel | Region Red |
| (9) | Selected | SER Spot | |
| | Region: Nucleus Region | SER Hole | |
| | | SER Edge | |
| | | SER Ridge | |
| | | SER Valley | |
| | | SER Saddle | |
| | | SER Bright | |
| | | SER Dark | |
| Calculate | Population: Nuclei | Method: STAR | Output Properties: |
| Morphology | Selected | Channel: Red | Nucleus Region |
| Properties | Selected | Symmetry | |
| (2) | Region: Nucleus Region | Threshold Compactness | |
| | | Axial | |
| | | Radial | |
| | | Profile | |
| | | Profile Inner Region: | |
| | | Nucleus | |
| | | Profile Width: 10 px | |
| | | Sliding Parabola | |
| | | Sliding Parabola | |
| | | Curvature: 10 | |
| | | Use for Center | |
| | | Texture SER | |
| | | Scale: 1 px | |
| | | Normalization by: Kernel | |
| | | SER Spot | |
| | | SER Hole | |
| | | SER Edge | |
| | | SER Ridge | |
| | | SER Valley | |
| | | SER Saddle | |
| | | SER Bright | |
| | | SER Dark | |
| Calculate | Population: Nuclei | Method: STAR | Output Properties: |
| Morphology | Selected | Channel: Red | Cytoplasm Region |
| Properties | Selected | Symmetry | |
| (3) | Region: Cytoplasm | Threshold Compactness | |
| | Region | Axial | |
| | | Radial | |
| | | Profile | |
| | | Profile Inner Region: | |
| | | Nucleus | |
| | | Profile Width: 10 px | |
| | | Sliding Parabola | |
| | | Sliding Parabola | |
| | | Curvature: 10 | |
| | | Use for Center | |
| | | Texture SER | |
| | | Scale: 1 px | |
| | | Normalization by: Kernel | |
| | | SER Spot | |
| | | SER Hole | |
| | | SER Edge | |
| | | SER Ridge | |
| | | SER Valley | |
| | | SER Saddle | |
| | | SER Bright | |
| | | SER Dark | |
| Calculate | Population: Nuclei | Method: STAR | Output Properties: |
| Morphology | Selected | Channel: Red | Membrane Region |
| Properties | Selected | Symmetry | |
| (4) | Region: Membrane | Threshold Compactness | |
| | Region | Axial | |
| | | Radial | |
| | | Profile | |
| | | Profile Inner Region: | |
| | | Nucleus | |
| | | Profile Width: 10 px | |
| | | Sliding Parabola | |
| | | Sliding Parabola | |
| | | Curvature: 10 | |
| | | Use for Center | |
| | | Texture SER | |
| | | Scale: 1 px | |
| | | Normalization by: Kernel | |
| | | SER Spot | |
| | | SER Hole | |
| | | SER Edge | |
| | | SER Ridge | |
| | | SER Valley | |
| | | SER Saddle | |
| | | SER Bright | |
| | | SER Dark | |

STATISTICAL DATA ANALYSIS: Transformation and Feature Selection. From two data sets containing 480 samples each (120 DMSO, 120 IKE, 120 RSL3, 120 STS) 1,473 features were generated and exported by the Columbus imaging software. The data sets were filtered for completeness, i.e., all features containing ‘not-a-number’ (NaN) were excluded from analysis, resulting in 1373 features. The data set generated first, was assigned to model discovery, the second data set to model validation. Features that were non-normally distributed in the discovery data according to Shapiro test for normality (p<0.05) were log-transformed (i.e., log(1+x) also known as two-parameter Box-Cox-transformation with lambda1=0 and lambda2=1), if the transformed data were closer to normality in terms of Shapiro-test p-value. Of all pairs of features that were highly correlated in the discovery data (i.e., absolute Pearson correlation coefficient of larger than 0.9) one member was excluded from analysis iteratively; starting with the feature participating in the largest number of correlations in the training data set for classifier discovery, which was preserved, all highly correlated features were removed from both data sets.

Classifier Discovery. Further feature pre-selection was conducted on the discovery data by logistic regression for pairwise classification among control, ferroptosis, and apoptosis using the lasso (least absolute shrinkage and selection operator) (Tibshirani, 2011). All features that were selected at least once in the pairwise logistic regressions were preserved in the training data set for classifier discovery, on which the classifier was trained. For classification a multinomial logistic regression model with the lasso was used, resulting in a signature for sample classification. Lambda.1se was used as criterion for selection of the optimal penalty parameter. The quality of this signature was determined in terms of accuracy of classification of the validation data, where true class membership is known. Importance of signature-features was estimated by the product of the standard deviation of the transformed feature in the discovery data and the coefficient in the regression model. All statistical calculations were conducted using R version 4.0.3, for lasso regression, glmnet package was used (Friedman et al. 2010).

Data Availability. The data underlying this study (raw data as txt files, R code Rmd file, and complete and intermediate Rdata files) are openly available in Columbia University Academic Commons at https://doi.org/10.7916/3hdp-9j07.

### DOCUMENTS CITED

Abrams, R. P., Carroll, W. L., and Woerpel, K. A. (2016). Five-Membered Ring Peroxide Selectively Initiates Ferroptosis in Cancer Cells. ACS Chem Biol 11, 1305-1312.
Aisen, P. (2004). Transferrin receptor 1. Int J Biochem Cell Biol 36, 2137-2143.
Alkan, S. S. (2004). Landmark—Monoclonal antibodies: the story of a discovery that revolutionized science and medicine. Nat Rev Immunol 4, 153-156.
Bertrand, R., Solary, E., O'Connor, P., Kohn, K. W., Pommier, Y. (1994) Induction of a Common Pathway of Apoptosis by Staurosporine. Experimental Cell Research 211, 314-321.
Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006). CellProfiler: image analysis software for identifying and quantifying cell phenotypes. Genome Biol 7, R100.
Chen, X., Gao, C., Yan, Y., Cheng, Z., Chen, G., Rui, T., Luo, C., Gao, Y., Wang, T., Chen, X. et al. (2021) Ruxolitinib exerts neuroprotection via repressing ferroptosis in a mouse model of traumatic brain injury. Exp Neurol. 342, 113762.
Chen, X., Kang, R., Kroemer, G., Tang, D. (2021) Broadening horizons: the role of ferroptosis in cancer. Nat Rev Clin Oncol. 18, 280-296.
Cheng, Y., Zak, O., Aisen, P., Harrison, S. C., Walz, T. (2004) Structure of the Human Transferrin Receptor-Transferrin Complex. Cell 116, 565-576.
Cox, J., and Mann, M. (2008). MaxQuant enables high peptide identification rates, individualized p.p.b.-range mass accuracies and proteome-wide protein quantification. Nat Biotechnol 26, 1367-1372.
Cox, J., Neuhauser, N., Michalski, A., Scheltema, R. A., Olsen, J. V., and Mann, M. (2011). Andromeda: A Peptide Search Engine Integrated into the MaxQuant Environment. J Proteome Res 10, 1794-1805.
Daniels, T. R., Bernabeu, E., Rodriguez, J. A., Patel, S., Kozman, M., Chiappetta, D. A., Holler, E., Ljubimova, J. Y., Helguera, G., and Penichet, M. L. (2012). The transferrin receptor and the targeted delivery of therapeutic agents against cancer. Bba-Gen Subjects 1820, 291-317.
Denton, D. and Kumar, S. (2015) Immunostaining Using an Antibody against Active Caspase-3 to Detect Apoptotic Cells in Drosophila. Cold Spring Harb Protoc 2015, 576-579.
Dixon, S. J., and Stockwell, B. R. (2019). The Hallmarks of Ferroptosis. Annu Rev Canc Biol 3, 35-54.
Dixon, S. J., Lemberg, K. M., Lamprecht, M. R., Skouta, R., Zaitsev, E. M., Gleason, C. E., Patel, D. N., Bauer, A. J., Cantley, A. M., Yang, W. S., et al. (2012). Ferroptosis: an iron-dependent form of nonapoptotic cell death. Cell 149, 1060-1072.
Dixon, S. J., Patel, D. N., Welsch, M., Skouta, R., Lee, E. D., Hayano, M., Thomas, A. G., Gleason, C. E., Tatonetti, N. P., Slusher, B. S., et al. (2014). Pharmacological inhibition of cystine-glutamate exchange induces endoplasmic reticulum stress and ferroptosis. Elife 3, e02523.
Dixon, S. J., Winter, G. E., Musavi, L. S., Lee, E. D., Snijder, B., Rebsamen, M., Superti-Furga, G., and Stockwell, B. R. (2015). Human Haploid Cell Genetics Reveals Roles for Lipid Metabolism Genes in Nonapoptotic Cell Death. ACS Chem Biol 10, 1604-1609.
Doll, S., Proneth, B., Tyurina, Y. Y., Panzilius, E., Kobayashi, S., Ingold, I., Irmler, M., Beckers, J., Aichler, M., Walch, A., et al. (2017). ACSL4 dictates ferroptosis sensitivity by shaping cellular lipid composition. Nat Chem Biol 13, 91-98.
Dolma, S., Lessnick, S. L., Hahn, W. C., and Stockwell, B. R. (2003). Identification of genotype-selective antitumor agents using synthetic lethal chemical screening in engineered human tumor cells. Cancer Cell 3, 285-296.
Drummen, G. P., van Liebergen, L. C., Op den Kamp, J. A., and Post, J. A. (2002). C11-BODIPY(581/591), an oxidation-sensitive fluorescent lipid peroxidation probe: (micro)spectroscopic characterization and validation of methodology. Free Radic Biol Med 33, 473-490.
Fan, Z., Cai, L., Wang, S., Wang, J., Chen, B. (2021) Baicalin Prevents Myocardial Ischemia/Reperfusion Injury Through Inhibiting ACSL4 Mediated Ferroptosis. Front Pharmacol. 12, 628988.
Feng, H., Schorpp, K., Jin, J., Yozwiak, C. E., Hoffstrom, B. G., Decker, A. M., Rajbhandari, P., Stokes, M. E., Bender, H. G., Csuka, J. M. et al. (2020) Transferrin Receptor Is a Specific Ferroptosis Marker. Cell Rep. 30, 3411-3423.
Feng, H. Z., and Stockwell, B. R. (2018). Unsolved mysteries: How does lipid peroxidation cause ferroptosis? Plos Biol 16.
Friedman, J., Hastie, T., Tibshirani, R. (2010) Regularization Paths for Generalized Linear Models via Coordinate Descent. Journal of Statistical Software 33, 1-22.
Friedmann Angeli, J. P., Krysko, D. V., Conrad, M. (2019) Ferroptosis at the crossroads of cancer-acquired drug resistance and immune evasion. Nat Rev Cancer 19, 405-414.
Gao, M., Monian, P., Quadri, N., Ramasamy, R., and Jiang, X. (2015). Glutaminolysis and Transferrin Regulate Ferroptosis. Mol Cell 59, 298-308.
Gaschler, M. M., Andia, A. A., Liu, H. R., Csuka, J. M., Hurlocker, B., Vaiana, C. A., Heindel, D. W., Zuckerman, D. S., Bos, P. H., Reznik, E., et al. (2018). FINO2 initiates ferroptosis through GPX4 inactivation and iron oxidation. Nature Chemical Biology 14, 507-+.
Hangauer, M. J., Viswanathan, V. S., Ryan, M. J., Bole, D., Eaton, J. K., Matov, A., Galeas, J., Dhruv, H. D., Berens, M. E., Schreiber, S. L., et al. (2017). Drug-tolerant persister cancer cells are vulnerable to GPX4 inhibition. Nature 551, 247-+.
Harrison, P. M., and Arosio, P. (1996). The ferritins: molecular properties, iron storage function and cellular regulation. Biochim Biophys Acta 1275, 161-203.
He, P., Ai, T., Yang, Z.-H., Wu, J., Han, J. (2020) Detection of necroptosis by phospho-MLKL immunohistochemical labeling. STAR Protoc. 2, 100251.
Jiang, X., Stockwell, B. R., Conrad, M. (2021) Ferroptosis: mechanisms, biology and role in disease. Nat Rev Mol Cell Biol. 22, 266-282.
Kotamraju, S., Chitambar, C. R., Kalivendi, S. V., Joseph, J., and Kalyanaraman, B. (2002). Transferrin receptor-dependent iron uptake is responsible for doxorubicin-mediated apoptosis in endothelial cells: role of oxidant-induced iron signaling in apoptosis. J Biol Chem 277, 17179-17187.
Larraufie, M.-H., Yang, W. S., Jiang, E., Thomas, A. G., Slusher, B. S., Stockwell, B. R. (2015) Incorporation of metabolically stable ketones into a small molecule probe to increase potency and water solubility. Bioorg Med Chem Lett. 25, 4787-4792.
Marques, O., Porto, G., Rema, A., Faria, F., Cruz Paula, A., Gomez-Lazaro, M., Silva, P., Martins da Silva, B., and Lopes, C. (2016). Local iron homeostasis in the breast ductal carcinoma microenvironment. BMC Cancer 16, 187.
Muller, T., Dewitz, C., Schmitz, J., Schroder, A. S., Brasen, J. H., Stockwell, B. R., Murphy, J. M., Kunzendorf, U., and Krautwald, S. (2017). Necroptosis and ferroptosis are alternative cell death pathways that operate in acute kidney failure. Cell Mol Life Sci 74, 3631-3645.
Schorpp, K., and Hadian, K. (2014). Small molecule screening at Helmholtz Zentrum Munchen—from biology to molecules. Combinatorial chemistry & high throughput screening 17, 266-271.
Shimada, K., Skouta, R., Kaplan, A., Yang, W. S., Hayano, M., Dixon, S. J., Brown, L. M., Valenzuela, C. A., Wolpaw, A. J., and Stockwell, B. R. (2016). Global survey of cell death mechanisms reveals metabolic regulation of ferroptosis. Nat Chem Biol 12, 497-503.
Skouta, R., Dixon, S. J., Wang, J., Dunn, D. E., Orman, M., Shimada, K., Rosenberg, P. A., Lo, D. C., Weinberg, J. M., Linkermann, A., et al. (2014). Ferrostatins inhibit oxidative lipid damage and cell death in diverse disease models. J Am Chem Soc 136, 4551-4556.
Sommer, C. and Gerlich, D. W. (2013) Machine learning in cell biology—teaching computers to recognize phenotypes. J Cell Sci 126, 5529-5539.
Sonabend, A. M., Yun, J., Lei, L., Leung, R., Soderquist, C., Crisman, C., Gill, B. J., Carminucci, A., Sisti, J., Castelli, M., et al. (2013). Murine cell line model of proneural glioma for evaluation of anti-tumor therapies. J Neurooncol 112, 375-382.
Stockwell, B. R., Angeli, J. P. F., Bayir, H., Bush, A. I., Conrad, M., Dixon, S. J., Fulda, S., Gascón, S., Hatzios, S. K., Kagan, V. E. et al. (2017) Ferroptosis: A Regulated Cell Death Nexus Linking Metabolism, Redox Biology, and Disease. Cell 171, 273-285.
Tampo, Y., Kotamraju, S., Chitambar, C. R., Kalivendi, S. V., Keszler, A., Joseph, J., and Kalyanaraman, B. (2003). Oxidative stress-induced iron signaling is responsible for peroxide-dependent oxidation of dichlorodihydrofluorescein in endothelial cells: role of transferrin receptor-dependent iron uptake in apoptosis. Circ Res 92, 56-63.
Tibshirani, R. (2011) Regression shrinkage and selection via the lasso: a retrospective. J R Stat Soc Series B Stat Methodol. 73, 273-282.
Viswanathan, V. S., Ryan, M. J., Dhruv, H. D., Gill, S., Eichhoff, O. M., Seashore-Ludlow, B., Kaffenberger, S. D., Eaton, J. K., Shimada, K., Aguirre, A. J., et al. (2017). Dependency of a therapy-resistant state of cancer cells on a lipid peroxidase pathway. Nature 547, 453-457.
Weiland, A., Wang, Y., Wu, W., Lan, X., Han, X., Li, Q., Wang, J. (2018) Ferroptosis and Its Role in Diverse Brain Diseases. Molecular Neurobiology 56, 4880-4893.
Wyllie, A. H., Kerr, J. F. R., Currie, A. R. (1980) Cell Death: The Significance of Apoptosis. Int Rev Cytol. 68, 251-306.
Yamada, S., Kumazawa, S., Ishii, T., Nakayama, T., Itakura, K., Shibata, N., Kobayashi, M., Sakai, K., Osawa, T., and Uchida, K. (2001). Immunochemical detection of a lipofuscin-like fluorophore derived from malondialdehyde and lysine. J Lipid Res 42, 1187-1196.
Yamanaka, K., Saito, Y., Sakiyama, J., Ohuchi, Y., Oseto, F., and Noguchi, N. (2012). A novel fluorescent probe with high sensitivity and selective detection of lipid hydroperoxides in cells. Rsc Adv 2, 7894-7900.
Yang, W. S. and Stockwell, B. R. (2008) Synthetic lethal screening identifies compounds activating iron-dependent, non-apoptotic cell death in oncogenic-RAS-harboring cancer cells. Chem Biol. 15, 234-245.
Yang, W. S., SriRamaratnam, R., Welsch, M. E., Shimada, K., Skouta, R., Viswanathan, V. S., Cheah, J. H., Clemons, P. A., Shamji, A. F., Clish, C. B. et al. (2014) Regulation of Ferroptotic Cancer Cell Death by GPX4. Cell 156, 317-331.
Yuan, H., Li, X. M., Zhang, X. Y., Kang, R., and Tang, D. L. (2016). Identification of
ACSL4 as a biomarker and contributor of ferroptosis. Biochem Bioph Res Co 478, 1338-1343.
Zhang, Y., Tan, H., Daniels, J. D., Zandkarimi, F., Liu, H., Brown, L. M., Uchida, K., O'Connor, O. A., and Stockwell, B. R. (2019). Imidazole Ketone Erastin Induces Ferroptosis and Slows Tumor Growth in a Mouse Lymphoma Model. Cell Chem Biol.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

1-37. (canceled)

38. An isolated monoclonal antibody or antigen binding fragment thereof, comprising a heavy chain variable region and a light chain variable region, comprising:
in the heavy chain variable region, the heavy chain complementarity determining regions set forth as SEQ ID NO: 3, SEQ ID NO: 4, and SEQ ID NO: 5, and
in the light chain variable region, the light chain complementarity determining regions set forth as SEQ ID NO: 6, SEQ ID NO: 7, and SEQ ID NO: 8.

39. The monoclonal antibody or antigen binding fragment of claim 38, which targets ectodomains of TfR1.

40. The monoclonal antibody or antigen binding fragment of claim 38, wherein the heavy chain variable region comprises the amino acid sequence set forth as SEQ ID NO: 1.

41. The monoclonal antibody or antigen binding fragment of claim 38, wherein the light chain variable region comprises the amino acid sequence set forth as SEQ ID NO: 2.

42. The monoclonal antibody or antigen binding fragment of claim 38, wherein the heavy and light chain variable regions comprise the amino acid sequences set forth as SEQ ID NO: 1 and SEQ ID NO: 2, respectively.

43. The monoclonal antibody or antigen binding fragment of claim 38, wherein the monoclonal antibody or antigen binding fragment comprises a human framework region.

44. The monoclonal antibody of claim 38, wherein the monoclonal antibody is an IgG.

45. The monoclonal antibody of claim 38.

46. The antigen binding fragment of claim 38.

47. The antigen binding fragment of claim 46, wherein the antigen binding fragment is a Fv, Fab, F(ab′)₂, scFV or a scFV₂ fragment.

48. An isolated nucleic acid molecule encoding the antibody or antigen binding fragment of claim 38.

49. The isolated nucleic acid molecule of claim 48, comprising nucleic acid sequences set forth as SEQ ID NOs: 9 and 10.

50. The isolated nucleic acid molecule of claim 48, comprising nucleic acid sequences set forth as SEQ ID NOs: 11 to 16.

51. A vector comprising the nucleic acid molecule of claim 48.

52. A host cell, comprising the nucleic acid molecule of claim 48 or a vector comprising the nucleic acid molecule.

53-63. (canceled)

64. A composition, comprising an effective amount of the antibody or antigen binding fragment of claim 38, or a nucleic acid molecule encoding the antibody or antigen binding fragment, and a pharmaceutically acceptable carrier.

65-77. (canceled)


SEQUENCE LISTING (25 sequences)

SEQ ID NO: 1 - protein, 118 aa, synthetic construct
  REGION 1..118: note = 3F3a heavy chain
EVLLQQSGTELVRPGALVKLSCKASGFNIQDLYIHWVKQRPEQGLEWIGWIDPETDNTIYDPKFQGKASITADTSSNTAYLQLSSLTSEDTAMYYCSTGLLQWYFDVWGAGTAVTVSS

SEQ ID NO: 2 - protein, 113 aa, synthetic construct
  REGION 1..113: note = 3F3a Kappa chain
DIVMTQSPSSLAMSIGQKVTMRCKSSQSLLNSYNQKNCLAWYQQKPGQSPKLLLYFVSTRESGVPDRFIGSGSGTDFTLTISSVQAEDLADYFCQQHYSTPLTFGAGTKLELK

SEQ ID NO: 3 - protein, 5 aa, synthetic construct
  REGION 1..5: note = 3F3a HCDR1
DLYIH

SEQ ID NO: 4 - protein, 20 aa, synthetic construct
  REGION 1..20: note = 3F3a HCDR2
WIDPETDNTIYDPKFQGKAS

SEQ ID NO: 5 - protein, 9 aa, synthetic construct
  REGION 1..9: note = 3F3a HCDR3
GLLQWYFDV

SEQ ID NO: 6 - protein, 17 aa, synthetic construct
  REGION 1..17: note = 3F3a LCDR1
KSSQSLLNSYNQKNCLA

SEQ ID NO: 7 - protein, 7 aa, synthetic construct
  REGION 1..7: note = 3F3a LCDR2
FVSTRES

SEQ ID NO: 8 - protein, 9 aa, synthetic construct
  REGION 1..9: note = 3F3a LCDR3
QQHYSTPLT

SEQ ID NO: 9 - DNA, 354 bp, synthetic construct
  misc_feature 1..354: note = 3F3a heavy chain
  source 1..354: mol_type = other DNA
gaggttctgctgcagcagtctgggactgagcttgtgaggccaggggccttagtcaagttgtcctgtaaagcttctggcttcaacattcaagacttatatatacactgggtgaaacagaggcctgaacagggcctggagtggattggatggattgatccagagactgataatactatatatgacccgaagttccagggcaaggccagtataacagcagacacatcctccaacacagcctacctacaactcagcagcctgacatctgaggacactgccatgtattactgttctacgggattactacagtggtacttcgatgtctggggcgctgggaccgcggtcaccgtctcctca

SEQ ID NO: 10 - DNA, 339 bp, synthetic construct
  misc_feature 1..339: note = 3F3a Kappa chain
  source 1..339: mol_type = other DNA
gacattgtgatgacacagtctccatcctccctggctatgtcgattggacagaaggtcactatgagatgcaagtccagtcagagccttttaaatagttacaatcaaaaaaactgtttggcctggtaccagcagaaaccaggacagtctcctaaacttctgctctactttgtatccactagggagtctggggtccctgatcgcttcataggcagtggatctgggacagatttcactcttaccatcagcagtgtgcaggctgaagacctggcagattacttctgtcagcaacattatagtactccgctcacgttcggtgctgggaccaagctggagctgaaa

SEQ ID NO: 11 - DNA, 15 bp, synthetic construct
  misc_feature 1..15: note = 3F3a HCDR1
  source 1..15: mol_type = other DNA
gacttatatatacac

SEQ ID NO: 12 - DNA, 69 bp, synthetic construct
  misc_feature 1..69: note = 3F3a HCDR2
  source 1..69: mol_type = other DNA
tggattggatggattgatccagagactgataatactatatatgacccgaagttccagggcaaggccagt

SEQ ID NO: 13 - DNA, 27 bp, synthetic construct
  misc_feature 1..27: note = 3F3a HCDR3
  source 1..27: mol_type = other DNA
ggattactacagtggtacttcgatgtc

SEQ ID NO: 14 - DNA, 51 bp, synthetic construct
  misc_feature 1..51: note = 3F3a LCDR1
  source 1..51: mol_type = other DNA
aagtccagtcagagccttttaaatagttacaatcaaaaaaactgtttggcc

SEQ ID NO: 15 - DNA, 21 bp, synthetic construct
  misc_feature 1..21: note = 3F3a LCDR2
  source 1..21: mol_type = other DNA
tttgtatccactagggagtct

SEQ ID NO: 16 - DNA, 27 bp, synthetic construct
  misc_feature 1..27: note = 3F3a LCDR3
  source 1..27: mol_type = other DNA
cagcaacattatagtactccgctcacg

SEQ ID NO: 17 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 18 - DNA, 23 bp, synthetic construct
  misc_feature 1..23: note = TfR1 primer FWD
  source 1..23: mol_type = other DNA
accattgtcatatacccggttca

SEQ ID NO: 19 - DNA, 23 bp, synthetic construct
  misc_feature 1..23: note = TfR1 primer RV
  source 1..23: mol_type = other DNA
caatagcccaagtagccaatcat

SEQ ID NO: 20 - DNA, 20 bp, synthetic construct
  misc_feature 1..20: note = GAPDH primer FWD
  source 1..20: mol_type = other DNA
ctccaaaatcaagtggggcg

SEQ ID NO: 21 - DNA, 20 bp, synthetic construct
  misc_feature 1..20: note = GAPDH primer RV
  source 1..20: mol_type = other DNA
atgacgaacatgggggcatc

SEQ ID NO: 22 - DNA, 875 bp, synthetic construct
  misc_feature 1..875: note = Target gene scFv for pcDNA3.1
  source 1..875: mol_type = other DNA
ggatccgccgccaccatgcacagctcagcactgctctgttgcctggtcctcctgactggggtgagggccgaagtcctcctccaacaatccggaacagaactcgttagacctggtgcactggtcaagctctcctgtaaagccagtggattcaacattcaggacctctacattcactgggtcaagcaacgcccagagcaagggctggagtggatcggctggattgatcccgagacagataacacaatctacgatcctaagttccagggtaaggcatccataactgccgacacaagcagtaatactgcatacctccagctgtcatcactcaccagcgaggatacagccatgtactactgttccactggactgctgcaatggtactttgatgtttggggagcagggactgccgttaccgtgtcctctggaggcggagggtccggtggaggaggctccggaggtggtggcagcgacatagtcatgacacagagcccaagcagcctggctatgagcatcgggcagaaggtgacaatgcggtgcaagtcctctcagagtctgctgaactcctacaatcagaagaattgtctggcctggtatcaacagaagccaggccaaagtcctaagctcctcctgtatttcgtttcaactcgcgagtctggtgtgcctgacagattcatcggcagtgggagcgggacagacttcactctcaccatcagtagtgtccaggctgaggacctggccgactatttctgtcaacaacattactcaactcctctgactttcggtgctggaaccaaactggagctgaagggcagcgagcagaaactgatttccgaagaggacctgagccaccatcaccaccatcactaatagaattc

SEQ ID NO: 23 - DNA, 812 bp, synthetic construct
  misc_feature 1..812: note = Target gene scFv for pET28a
  source 1..812: mol_type = other DNA
ccatggaagtgctgctgcaacaaagcggcaccgaactggtacgtccgggtgctctggtgaaactgtcttgtaaagcatctggtttcaacatccaggacctgtacattcactgggttaaacagcgtccggaacaaggcctggaatggatcggttggatcgacccggaaactgacaacactatctacgacccgaaattccaaggtaaagcaagcattaccgcagacacctcttccaacaccgcgtacctgcaactgtcctctctgacctctgaagataccgcaatgtactactgtagcactggtctgctgcaatggtactttgatgtttggggtgccggtactgcggtgactgtttcctctggtggcggtggttctggcggtggtggttctggtggtggtggctctgacattgtgatgacccagtctccgagcagcctggcgatgtccatcggtcagaaggttactatgcgctgcaagtcctcccagtccctgctgaactcctataaccagaagaattgtctggcttggtatcagcagaaaccgggtcaatctccgaagctgctgctgtactttgtttctactcgtgagtccggtgtaccagatcgctttatcggttccggttctggcaccgacttcaccctgaccatcagctccgttcaggcggaggatctggccgactatttctgccagcaacactatagcactccgctgacctttggtgctggcaccaaactggaactgaagggttctgagcagaaactgattagcgaagaggatctgtctcatcaccaccaccatcattaatagctcgag

SEQ ID NO: 24 - DNA, 6274 bp, synthetic construct
  misc_feature 1..6274: note = pcDNA3.1(+)-scFv full sequence
  source 1..6274: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatgcacagctcagcactgctctgttgcctggtcctcctgactggggtgagggccgaagtcctcctccaacaatccggaacagaactcgttagacctggtgcactggtcaagctctcctgtaaagccagtggattcaacattcaggacctctacattcactgggtcaagcaacgcccagagcaagggctggagtggatcggctggattgatcccgagacagataacacaatctacgatcctaagttccagggtaaggcatccataactgccgacacaagcagtaatactgcatacctccagctgtcatcactcaccagcgaggatacagccatgtactactgttccactggactgctgcaatggtactttgatgtttggggagcagggactgccgttaccgtgtcctctggaggcggagggtccggtggaggaggctccggaggtggtggcagcgacatagtcatgacacagagcccaagcagcctggctatgagcatcgggcagaaggtgacaatgcggtgcaagtcctctcagagtctgctgaactcctacaatcagaagaattgtctggcctggtatcaacagaagccaggccaaagtcctaagctcctcctgtatttcgtttcaactcgcgagtctggtgtgcctgacagattcatcggcagtgggagcgggacagacttcactctcaccatcagtagtgtccaggctgaggacctggccgactatttctgtcaacaacattactcaactcctctgactttcggtgctggaaccaaactggagctgaagggcagcgagcagaaactgatttccgaagaggacctgagccaccatcaccaccatcactaatagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 25 - DNA, 6037 bp, synthetic construct
  misc_feature 1..6037: note = pET28a(+)-scFv full sequence
  source 1..6037: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagctattaatgatggtggtggtgatgagacagatcctcttcgctaatcagtttctgctcagaacccttcagttccagtttggtgccagcaccaaaggtcagcggagtgctatagtgttgctggcagaaatagtcggccagatcctccgcctgaacggagctgatggtcagggtgaagtcggtgccagaaccggaaccgataaagcgatctggtacaccggactcacgagtagaaacaaagtacagcagcagcttcggagattgacccggtttctgctgataccaagccagacaattcttctggttataggagttcagcagggactgggaggacttgcagcgcatagtaaccttctgaccgatggacatcgccaggctgctcggagactgggtcatcacaatgtcagagccaccaccaccagaaccaccaccgccagaaccaccgccaccagaggaaacagtcaccgcagtaccggcaccccaaacatcaaagtaccattgcagcagaccagtgctacagtagtacattgcggtatcttcagaggtcagagaggacagttgcaggtacgcggtgttggaagaggtgtctgcggtaatgcttgctttaccttggaatttcgggtcgtagatagtgttgtcagtttccgggtcgatccaaccgatccattccaggccttgttccggacgctgtttaacccagtgaatgtacaggtcctggatgttgaaaccagatgctttacaagacagtttcaccagagcacccggacgtaccagttcggtgccgctttgttgcagcagcacttccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca